(19) United States
(12) Patent Application Publication
(10) Pub. No.: US 2026/0034356 A1
(43) Pub. Date: Feb. 5, 2026

(54) Transcranial Electrical Stimulation in Stroke Early After Onset

(71) Applicant: THE REGENTS OF THE UNIVERSITY OF CALIFORNIA, Oakland, CA (US)

(72) Inventors: Mersedeh Bahr Hosseini, West Hollywood, CA (US); Jeffrey L. Saver, Oakland, CA (US); Marom Bikson, Oakland, CA (US)

(21) Appl. No.: 19/100,814

(22) PCT Filed: Aug. 4, 2023
(86) PCT No.: PCT/US2023/071650

§ 371 Date: Feb. 3, 2025

Related U.S. Application Data
(60) Provisional application No. 63/370,428, filed on Aug. 4, 2022.

(51) Int. Cl. A61N 1/36; A61N 1/04

(52) U.S. Cl. CPC A61N 1/36025; A61N 1/0456; A61N 1/0476; A61N 1/0484; A61N 1/36034

(57) ABSTRACT

Disclosed is a non-invasive system and related methods to deliver electrical current to ischemic brain tissue rapidly with minimal set up, to treat acute ischemic stroke via direct cytoprotection and by collateral blood flow enhancement promoting recanalization of occluded vessel. In one embodiment, the system includes a cap with plurality of electrodes which are operable in combination to deliver an electrical current when in contact onto skin of the head region of the subject. In one embodiment, the electrical current delivery is individualized to target each individual's ischemic tissue only. In one embodiment, the electrode positioning can be selected from pre-determined electrode montages according to the location of the ischemia and vessel occlusion site. In one embodiment, the system can be applied as a standalone treatment or adjunct to reperfusion therapies to salvage the at-risk ischemic tissue (aka penumbra) and improve patient outcomes.

### CROSS-REFERENCE TO RELATED APPLICATIONS

This application claims priority to U.S. provisional application No. 63/370,428 filed on Aug. 4, 2022, incorporated herein by reference in its entirety.

### BACKGROUND OF THE INVENTION

Transcranial direct current stimulation (tDCS) is a non-invasive neuromodulatory technique that applies a weak electrical current to the brain via scalp electrodes. tDCS has been investigated in a variety of chronic neurological and neuropsychiatric disorders such as depression, traumatic brain injury and stroke rehabilitation with promising benefits and an excellent safety profile. Notably, tDCS has been shown to carry a strong neuroprotective effect in animal models of acute ischemic stroke (AIS) due to large vessel occlusion (LVO).

Stroke is a leading cause of mortality and morbidity across the world. Treatments for acute ischemic stroke are limited to reperfusion therapies, including intravenous thrombolysis (IV lytics) and endovascular therapy (EVT). However, the rate of reperfusion with IV lytics remains low ranging from 15% to 50%, and patients' outcomes even with EVT remain suboptimal, with only 20-25% achieving a disability-free outcome. Complementary neuroprotective, vasoprotective, and collateral enhancement strategies can preserve cells and the neurovascular units until orthograde reperfusion is achieved by reperfusion therapies, particularly in AIS patients in whom reperfusion therapy is delayed due to hospital-to-hospital transfer. Furthermore, these agents may protect against reperfusion injury, and attenuate secondary injury cascades that persist despite reperfusion. Importantly as well, even in the modern thrombectomy era, not all patients with LVO are treated with reperfusion therapies. For example, many patients arrive outside of the therapeutic window for IV lytics and in some patients with LVOs, EVT is not technically performable due to unfavorable angioarchitectural features or in other LVO patients, the risk of intervention is judged too great, due to various reasons such as the occlusion is too distal to be reached with stent-retrievals (medium and distal vessel occlusions), temporally advanced or large core infarct, or severe cardiorespiratory and other medical comorbidities.

Thus, there is a need for both complementary and standalone therapies. While many past attempts at developing neuroprotective therapies have been disappointing, the great preponderance were pharmacologic agents that affected only one or two molecular ischemic pathways and were administered intravenously, reducing delivery to brain areas with low blood flow, and, even there, requiring passage through the blood-brain barrier (BBB). In contrast, in pre-clinical acute ischemic stroke models, cathodal tDCS (C-tDCS) has been shown to have widely pleiotropic neuroprotective molecular mechanisms of action and can be delivered non-invasively to the salvageable ischemic tissue beyond the occlusive thrombus despite reduced blood flow. Furthermore, the high-definition (HD) electrode positioning allows refinement of the electrical current shape and location to be delivered only to the ischemic tissue in an individualized manner. Given the promising results in acute stroke models, translational studies in acute human ischemic stroke patients are needed, beginning with dose-escalation feasibility and safety trials. The present invention meets this need.

### SUMMARY OF THE INVENTION

In one aspect, the present invention provides a system and method for delivering non-invasive rapidly deployable individualized therapy for acute ischemic strokes comprising the steps of: performing acute stroke imaging to localize the individual's therapeutic target ischemic brain region, positioning the electrical stimulation system comprising a cap with a plurality of openings, a plurality of electrode holders and a plurality of electrodes onto a subject's head, wherein the cap is configured with plurality of electrodes that are affixed to, embedded or otherwise integrated into plurality of electrode holders that are attached to the plurality of openings on the cap; selection of the plurality of electrodes in a specific montage which corresponds to the identified therapeutic target ischemic region; and providing electrical current to the plurality of selected electrodes to salvage the individual's threatened brain tissue by enhancing collateral perfusion and by direct cytoprotection.

In one aspect, the present invention provides a method of providing non-invasive individualized therapy for acute ischemic strokes comprising the steps of: positioning an electrical stimulation device comprising a cap comprising: a plurality of openings, a plurality of electrode holders and a plurality of electrodes onto a subject's head, wherein the plurality of electrodes are affixed to, embedded or otherwise integrated into plurality of electrode holders that is attached to the plurality of openings on the cap; positioning the plurality of electrodes in a specified montage which corresponds to a therapeutic target ischemic region identified on perfusion hemodynamic imaging; and providing a direct electrical current to the plurality of electrodes to salvage the subject's threatened brain tissue by enhancing collaterals due to its vasodilatory properties enhancing cerebral perfusion and promoting recanalization, and inhibiting the peri-infarct excitotoxicity.

In another aspect, a method of providing therapy for acute ischemic strokes comprising the steps of: positioning an electrical stimulation device comprising a cap comprising: a plurality of openings, a plurality of electrode holders and a plurality of electrodes onto a subject's head, wherein the plurality of electrodes are affixed to, embedded or otherwise integrated into plurality of electrode holders that is attached to the plurality of openings on the cap; positioning the plurality of electrodes in a specified montage which corresponds to a therapeutic target region; and providing a direct electrical current to the plurality of electrodes to salvage the subject's brain via reduction of an ischemic region volume thereby enhancing collaterals and promoting recanalization.

In another aspect, a method of providing therapy for acute ischemic strokes comprising the steps of: positioning an electrical stimulation device comprising a cap comprising: a plurality of openings, a plurality of electrode holders and a plurality of electrodes onto a subject's head, wherein the plurality of electrodes are affixed to, embedded or otherwise integrated into plurality of electrode holders that is attached to the plurality of openings on the cap; positioning the plurality of electrodes in a specified montage which corresponds to a therapeutic target region; and providing a direct electrical current with a stimulation intensity ranging from 1 mA to 4 mA for 10 minutes to 60 minutes to the plurality of electrodes

In another aspect, a method of treating a patient with acute ischemic strokes comprises the steps of: positioning an electrical stimulation system comprising a cap configured with a plurality of electrodes onto a patient's head; and activating the plurality of electrodes in a specified montage which corresponds to a therapeutic target region, wherein activating includes providing a direct electrical current to the plurality of electrodes to salvage the patient's brain.

In one embodiment, the therapeutic target region is an ischemic tissue comprising the at-risk salvageable brain tissue also known as penumbral region. In one embodiment, the target region is identified with the assistance of stroke imaging comprising at least one brain imaging, vessel study, and perfusion hemodynamic imaging, MRI, and CT. In one embodiment, the target region is identified according to a vessel study showing a cortical vessel blockage if hemodynamic perfusion imaging is not available. In one embodiment, the target region is identified according to patients' clinical presentation consistent with cortical vessel occlusion if hemodynamic perfusion imaging nor vessel imaging are available. In one embodiment, the plurality of electrodes are arranged in a 4×1 array, wherein there is at least one central cathodal electrode surrounded by at least four return electrodes arranged around the center cathodal electrode.

In one embodiment, the montage is selected from the group consisting of: middle cerebral artery (MCA) M1 branch, MCA inferior branch (M2-I), MCA superior branch (M2-S), Anterior Cerebral artery (ACA), Posterior Cerebral Artery (PCA), Posterior Inferior Cerebellar Artery (PICA), and Internal Carotid artery (ICA) territory ischemia montage. In one embodiment, the ACA montage is positioned proximate to the longitudinal fissure of the patient's brain, and proximate to the anterior portion of the patient's brain. In one embodiment, the ACA montage comprises anodes over FPz, FCz, FC3, FP1, and cathode over F1 electrode locations.

In one embodiment, the MCA branch montage is positioned proximate to a central location between to the longitudinal fissure and outer edge of the patient's brain, and proximate to a central location between anterior and posterior portions of the patient's brain. In one embodiment the MCA branch montage comprises anodes over F3, Cz, P3, T7 and cathode over C3 electrode locations.

In one embodiment, the M2-S branch montage is positioned proximate to a central location between to the longitudinal fissure and outer edge of the patient's brain, and proximate the anterior portion of the patient's brain. In one embodiment, the M2-S branch montage comprises anodes over C1, F1, F7, T7 and cathode over FC3 electrode locations.

In one embodiment, the M2-I branch montage is positioned proximate to a central location between to the longitudinal fissure and outer edge of the patient's brain, and proximate the posterior portion of the patient's brain. In one embodiment, the M2-I branch montage comprises anodes over C3, P3, T7, P7 and cathode over CP5 electrode locations.

In one embodiment, the PCA montage is positioned proximate to the longitudinal fissure of the patient's brain, and proximate to the posterior portion of the patient's brain. In one embodiment, the PCA montage comprises anodes over Pz, Iz, PO9, P3 and cathode over O1 electrode locations.

In one embodiment, the PICA montage is positioned proximate to the outer edge of the patient's brain, and proximate to the posterior portion of the patient's brain. In one embodiment, the PICA montage comprises anodes over O1, P7, Ex1, EX5 and cathode over PO9 electrode locations.

In one embodiment, the ICA montage spans from proximate to the longitudinal fissure to proximate to the outer edge of the patient's brain, and proximate the anterior portion of the patient's brain. In one embodiment, the ICA montage comprises anodes over the Fpz,Cz,CP5,F9 and cathode over F3 electrode locations.

In one embodiment, the electrical current is applied with a stimulation intensity ranging between about 1-4 mA. In one embodiment, the direct electrical current is applied with a stimulation intensity of 1 and 2 mA. In one embodiment, the direct electrical current is applied at a pre-determined duty cycle, wherein the cycle is initiated for a given duration at predetermined intervals. In one embodiment, the direct electrical current is applied for a duration ranging between 10 to 60 minutes continuously. In one embodiment, the direct electrical current is applied for a duration of 20 minutes continuously. In one embodiment, the direct electrical current is applied for up to three 20-minute cycles. In one embodiment, the direct electrical current level is initially provided according to a current ramp up having a selected duration and current application is terminated according to a current ramp down having a selected duration. In one embodiment, the selected duration of the current ramp up or down is ranging between 10 seconds to 10 minutes.

In one embodiment, the ischemic region volume is reduced by 46% to 100%. In one embodiment, the enhanced collateral comprises an increase of 40% to 110% of quantitative relative cerebral blood volume (qrCBV).

In one embodiment a rate of the recanalization is 80%.

In one embodiment, the therapy results in salvage of threatened penumbral tissue. In one embodiment, the effect of the therapy on collateral perfusion is measured and quantified using cerebral blood volume (CBV)/cerebral blood flow (CBF) maps of perfusion hemodynamic imaging and evidence of cerebrospinal fluid (CSF) hyperintensity marker (CSF-HM) on brain MRI using Fluid Attenuated Inversion Recovery (FLAIR) sequence indicative of the vasodilatory effect of electrical current resulting in collateral enhancement and BBB modulation. In one embodiment, there is a dose-response effect with higher intensities stimulation resulting in a greater enhancement of CBV and CBF than lower intensities.

In another aspect, a system for providing therapy for acute ischemic strokes comprising: an electrical stimulation device comprising a cap comprising: a plurality of openings; and a plurality of electrode holders and a plurality of electrodes configured to place on a subject's head; wherein the plurality of electrodes are affixed to, embedded or otherwise integrated into plurality of electrode holders that is attached to the plurality of openings on the cap; and wherein the plurality of electrodes are arranged in a specified montage which corresponds to a therapeutic target region; and a computing system communicatively connected to the electrical stimulation device, comprising a processor and a non-transitory computer-readable medium with instructions stored thereon, which when executed by the processor, perform steps comprising: providing a direct electrical current to the plurality of electrodes to salvage the subject's brain via reduction of an ischemic region volume thereby enhancing collaterals and promoting recanalization.

In another aspect, a system for providing therapy for acute ischemic strokes comprises: wearable cap including an electrode array; a stimulator electrically connected to the electrode array for providing electrical stimulation via the electrode array; and a user interface and a controller for controlling stimulation parameters. In one embodiment, the system further includes a computing device.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1a depicts an exemplary electrical stimulation system of the present invention.

FIG. 1B depicts an exemplary electrical stimulation device of the present invention.

FIG. 1C depicts an exemplary computing device in which aspects of the present invention may be practiced.

FIG. 2 is a flowchart depicting an exemplary method of treating a subject having acute ischemic stroke with the system of the present invention.

FIG. 3 depicts a schematic showing the events leading to ischemic brain.

FIG. 4 depicts a forest plot showing the neuroprotective effect of electrical stimulation of central nervous system across 21 pre-clinical experiments.

FIG. 5 depicts models of intra-cranial current flow during electrical stimulation concentrated by the vasculature and vascular response to transcranial electrical stimulation. The electrical field concentrates in the cerebrospinal fluid of subarachnoid space where pial vasculature and blood-brain barrier reside.

FIG. 6 depicts vasodilatory response of pial arteries and penetrating arterioles with multilayered smooth muscle cells results from electrically induced release of potent vasodilators from perivascular nerve endings and the endothelial lining.

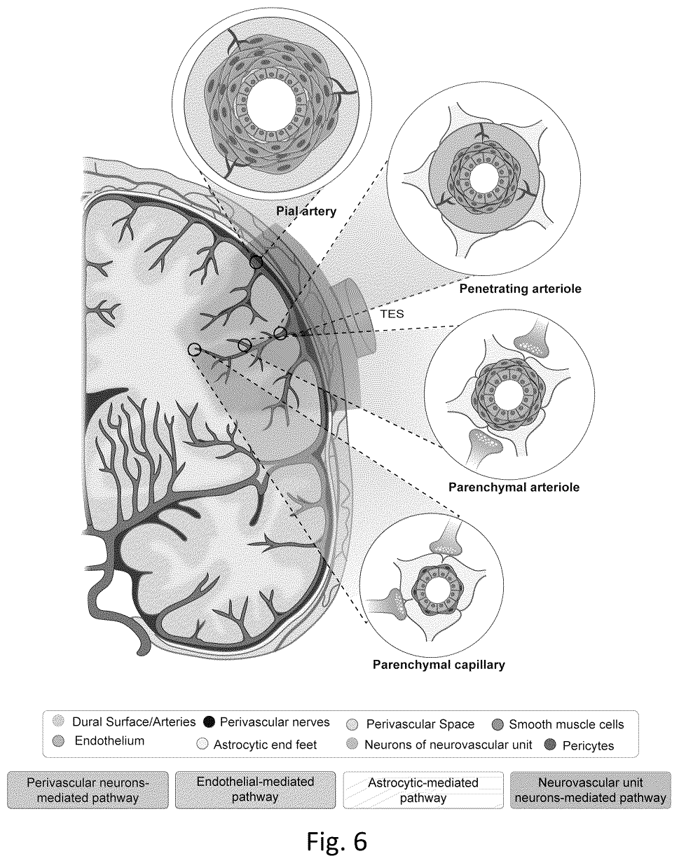

FIG. 7 Four cellular elements have been identified driving the direct primary response to tDCS: 1) Perivascular neuron-mediated response resulting from stimulation of perivascular nerves releasing vasoactive peptides (Calcium-gene Related Peptide (CGRP), Vasoactive Intestinal Peptide (VIP), and Nitric Oxide (NO); 2) Endothelium-mediated response involving release of vasoactive peptides (NO), activation of ion channels (K^{+ATP}), and changes in BBB permeability; 3) Astrocyte-mediated response from stimulated astrocytes releasing vasoactive substances in response to electrical stimulation independent of primary neural activity of NVC; and 4) Neurons of Neurovascular Unit (NVU)-mediated response leading to traditional neurovascular coupling.

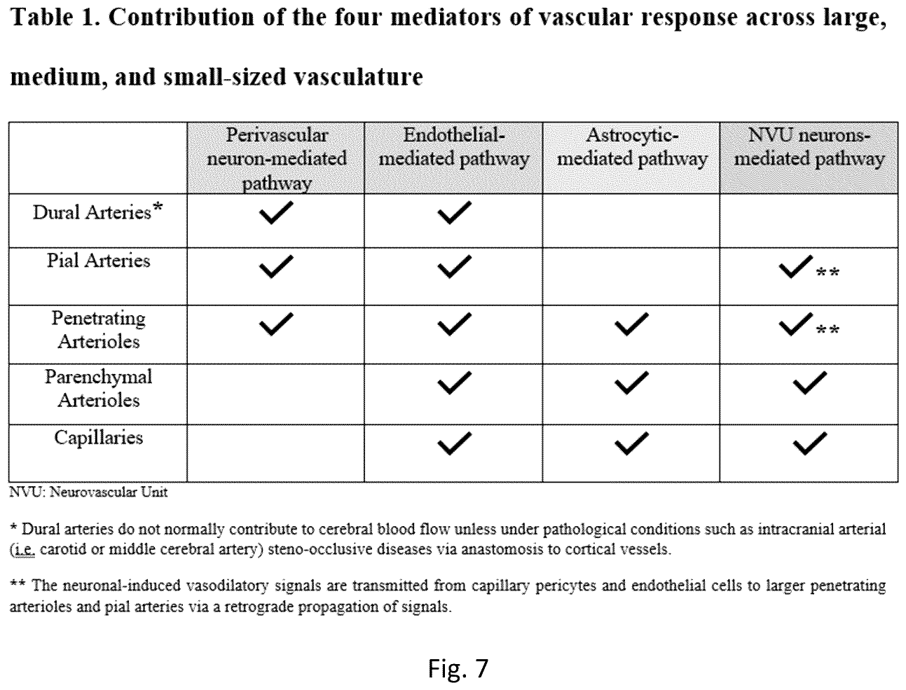

FIGS. 8A-8D depict an example of HD electrode positioning in a 4 to 1 configuration in a patient with left M1 occlusion. FIG. 8A depicts the reference electrodes (anode-blue) are positioned on F3, T3, Cz and P3 and the center electrode (cathode-red) is positioned over the C3 (central sulcus). FIG. 8B depicts the computational modeling of the electrical field, concentrated over the MCA territory. FIG. 8C depicts the electrode positioning on the tDCS HD cap, the schematic of the tDCS stimulator connected to the high-definition (HD) interface, and the Soterix™ HD tDCS unit (stimulator+interface) that is used in this study. This stimulator connects to the adjustable cap. FIG. 8D depicts the penumbral region on the perfusion MRI of the patient with L MCA occlusion.

FIGS. 9A-9G depict computational modeling of seven electrical fields and their corresponding electrode positioning covering Middle Cerebral Artery (MCA) main branch (MCA-M1), MCA Superior branch (MCA-M2-S), MCA Inferior branch (MCA-M2-I), Posterior Cerebral Artery (PCA), Anterior Cerebral Artery (ACA), Posterior Inferior Cerebellar Artery (PICA), ICA (internal carotid artery) territories according to the arterial occlusion site and location of ischemia. FIG. 9A depicts electrical filed covering the MCA-M1 branch territory, wherein anodes are over F3, Cz, P3, T7 and cathode is over C3. FIG. 8B depicts coverage of the MCA-M2S territory, wherein anodes are over C1, F1, F7, T7 and cathode is over FC3. FIG. 9C depicts coverage of the MCA-M2I territory, wherein anodes are over C3, P3, T7, P7 and cathode is over CP5. FIG. 9D depicts coverage of the ACA territory, wherein the anodes are over FPz, FCz, FC3, FP1 and cathode is over F1. FIG. 8E depict coverage of the PCA territory, wherein anodes are over Pz, Iz, PO9, P3 and cathode is over O1. FIG. 9F depicts coverage of the PICA territory, wherein anodes are over O1, P7, Ex1, EX5 and cathode is over PO9. FIG. 9G depicts coverage of the ICA territory, wherein anodes are over the Fpz, Cz, CP5,F9 and cathode is over F3.

FIG. 10 depicts the six escalating tiers. Blue represents 1 mA and purple shows 2 mA of HD C-tDCS. Symptomatic ICH (SICH) in none of the three patients at each tier results in escalation. However, SICH in one patient, 3 more patients and 1 sham is enrolled at the same tier.

FIG. 11 depicts the technician-filled tolerability form. The selected items are based on the most commonly reported adverse effects associated with tDCS.

FIG. 12 depicts seven different montages (configuration with 4 reference electrodes and one center electrode) based on 10-20 EEG electrode positioning map to cover seven vascular territories at risk of infarction in acute stroke. After detection of the ischemic tissue containing the at-risk tissue also known as penumbra on perfusion hemodynamic MRI or CT, the appropriate montage is selected. The goal is to deliver the electrical current to each individual's ischemic tissue only.

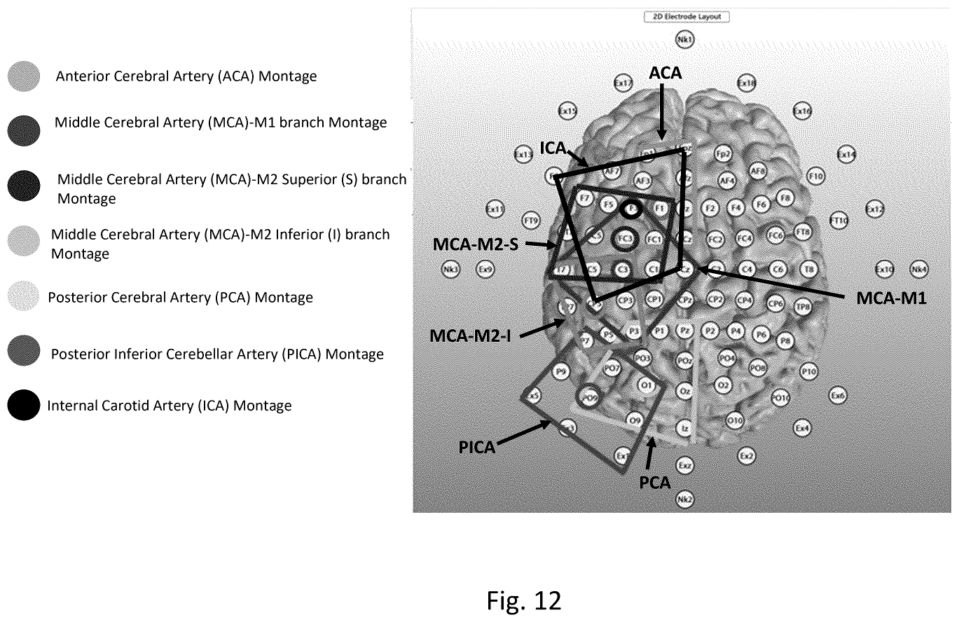

FIG. 13 depicts a 67-year-old female with left inferior division middle cerebral artery (MCA-M2I) stroke with persistent penumbra (circled the ischemic tissue). She received 20 minutes of 1 mA HD C-tDCS to the posterior right temporal region with M2-I montage shown on the mannequin. The computational modeling shows the specificity of the coverage provided to the brain tissue at risk (circled) over the posterior temporal region.

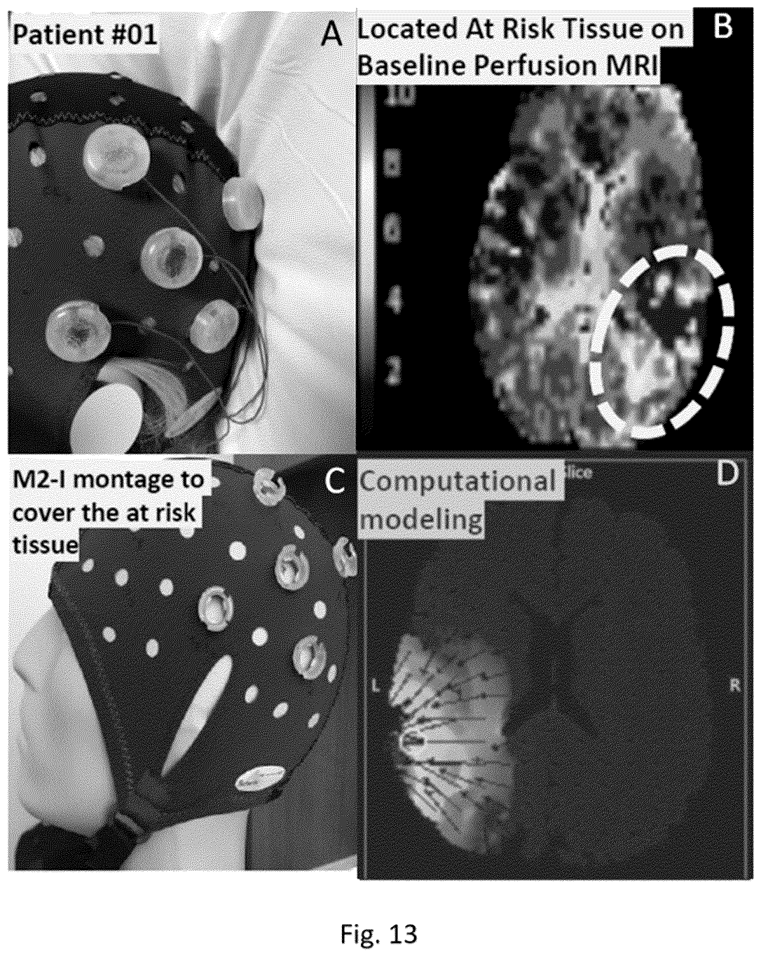

FIGS. 14A-14D depict a demonstration of HD C-tDCS effect on the occluded left inferior MCA-M2I branch and cerebral blood flow of posterior temporal region (circled) from baseline (first row) to 2 hours (2^{nd} row) to 24 hours (3^{rd} row). FIG. 14A depicts MR angiography. Arrows suggest improvement of anterograde blood flow from baseline to 24 hr post stimulation indicative of recanalization of the occluded left inferior MCA-M2I branch. FIG. 14B depicts Tmax map (time takes for the contrast to pass through arterial, capillary and then venous system). Green region is indicative of a delay in contrast passage due to vessel blockage. There is evidence of resolution of the delay from baseline to 24 hr. FIG. 14C and FIG. 14D depict relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is evidence of improvement in the CBF particularly at 24 hr shown in the circle.

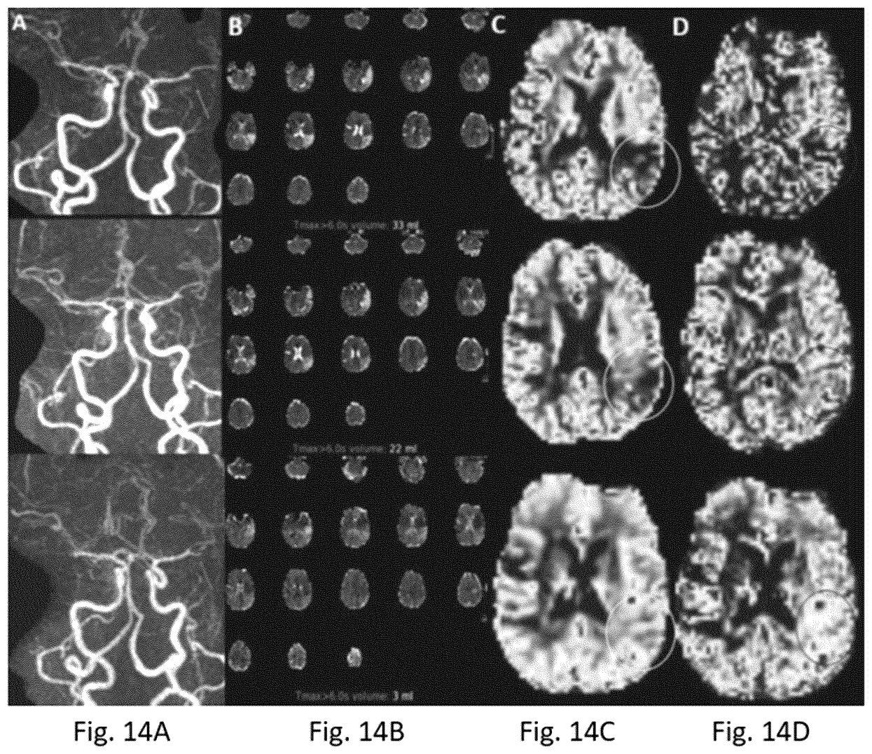

FIGS. 15A-15C depict diffusion weighted image (DWI), fluid attenuated inversion recovery (FLAIR), and gradient recall echo (GRE) MRI sequences at baseline (first row), 2-4 hours post-stimulation (second row), and 24-30 hours post-stimulation. Arrows indicate M2I slow flow which alleviated post-stimulation suggestive of recanalization.

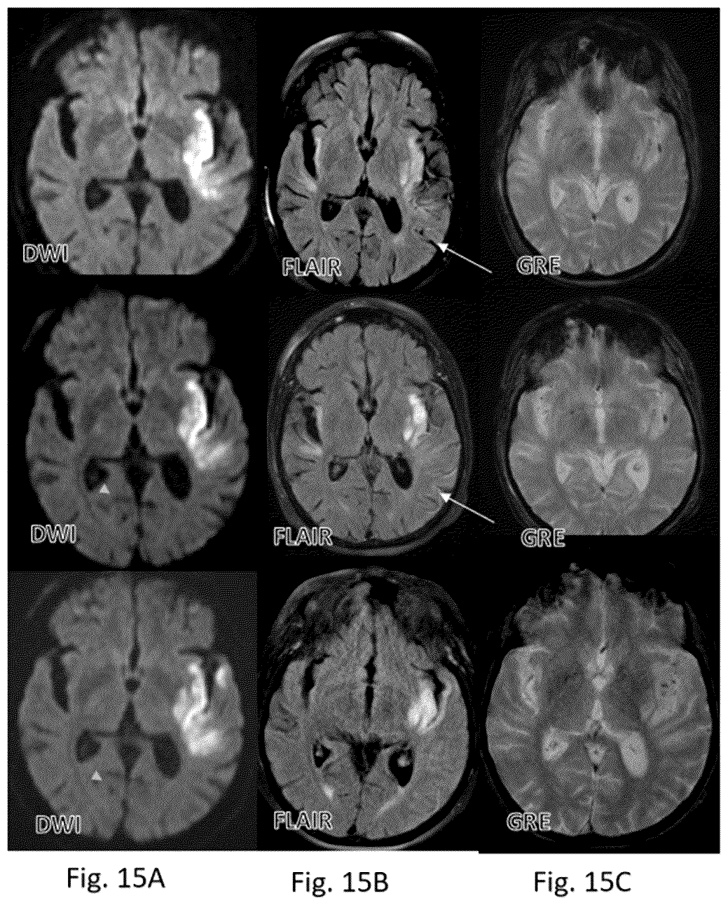

FIG. 16 depicts a 72-year-old male presented with right homonymous hemianopsia and ataxia due to a left posterior cerebral artery (PCA) occlusion with persistent penumbra (circled the ischemic tissue). He received 20 minutes of 1 mA of HD C-tDCS with PCA montage shown on the mannequin. The computational modeling shows the specificity of the coverage provided to the brain tissue at risk (circled) over the occipital region.

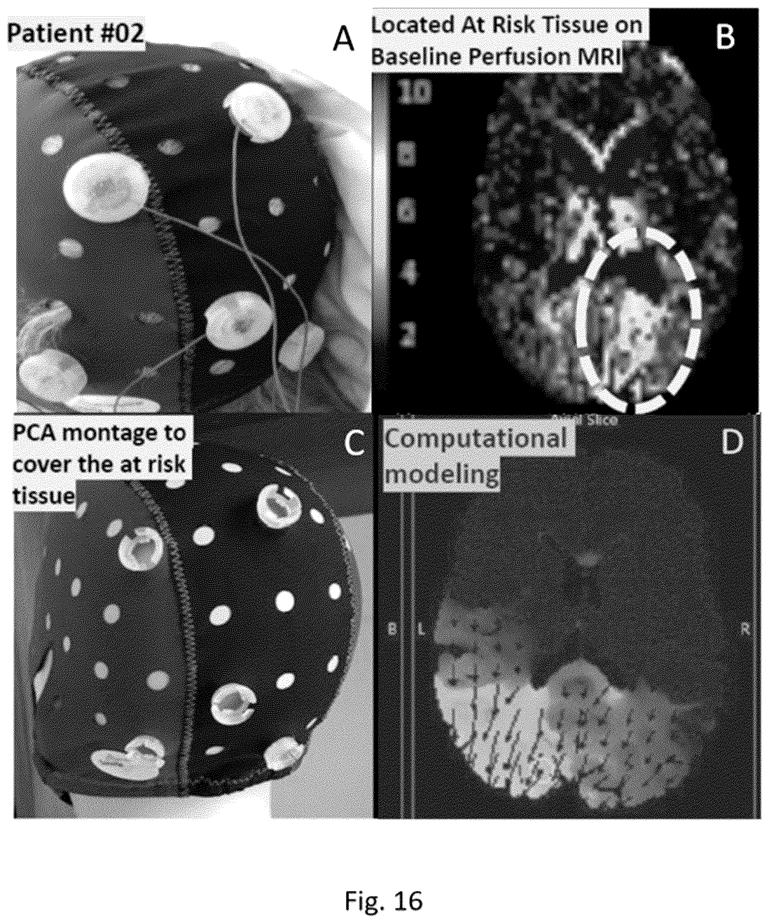

FIGS. 17A-17D depict a demonstration of HD C-tDCS effect on the occluded left PCA and cerebral blood flow and volume of left occipital region (circled) from baseline (first row) to 2 hours (2^{nd} row) to 24 hours (3^{rd} row). FIG. 17A depicts MR angiography. Arrows suggest improvement of anterograde blood flow from baseline to 24 hr post stimulation indicative of recanalization of the occluded left PCA. FIG. 17B depicts Tmax map (time takes for the contrast to pass through arterial, capillary and then venous system). Green region is indicative of a delay in contrast passage due to vessel blockage. There is evidence of resolution of the delay from baseline to 24 hr. FIG. 17C and FIG. 17D depicts relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is evidence of improvement in the CBF/CBV particularly at 24 hr shown in the circle.

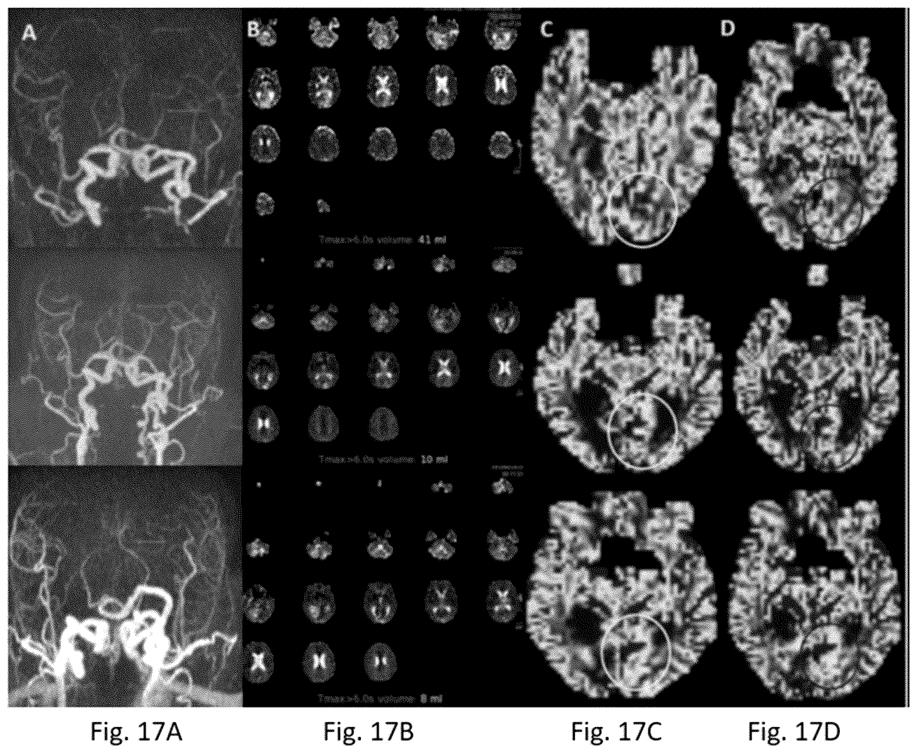

FIGS. 18A-18C depict diffusion weighted image (DWI), fluid attenuated inversion recovery (FLAIR), and gradient recall echo (GRE) MRI sequences at baseline (first row), 2-4 hours post-stimulation (second row), and 24-30 hours post-stimulation. Yellow Arrows indicate PCA slow flow which alleviated post-stimulation suggestive of recanalization. Orange arrows indicate CSF hyperintensity marker over the bilateral occipital regions.

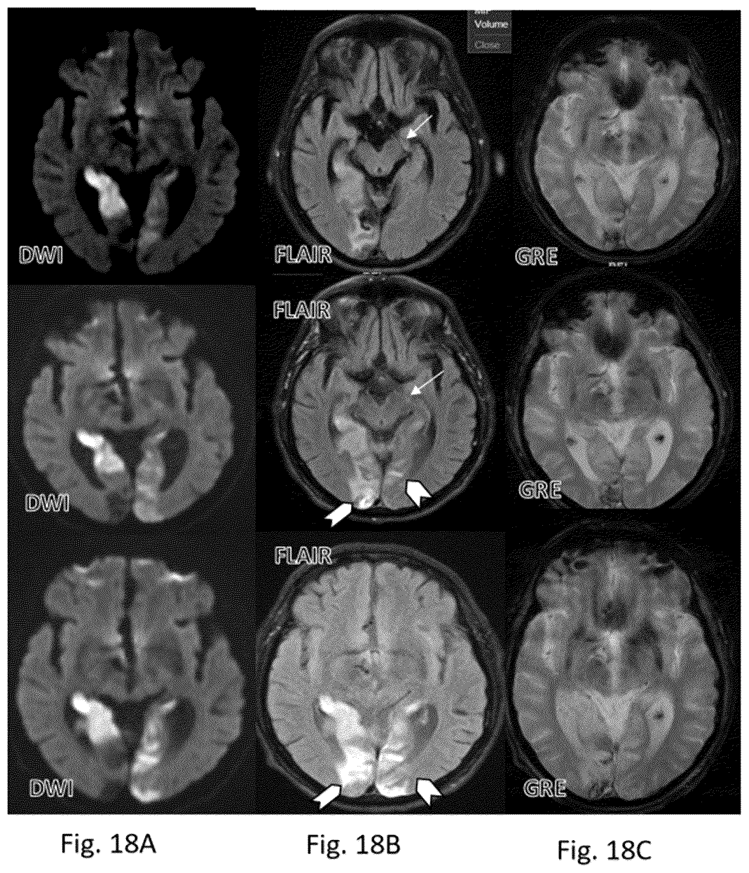

FIG. 19. depicts a 65-year-old female presented with global aphasia due to distal left temporo-parietal branch of middle cerebral artery occlusion (MCA-M3) with persistent penumbra (circled the ischemic tissue). She received 20 minutes of SHAM HD C-tDCS (sham with 30 seconds of ramp up at the beginning and 30 seconds of ramp down at the end of the stimulation) to the posterior right temporal region with M2-I montage shown on the mannequin. The computational modeling shows the specificity of the coverage provided to the brain tissue t risk (circled) over the posterior temporal region.

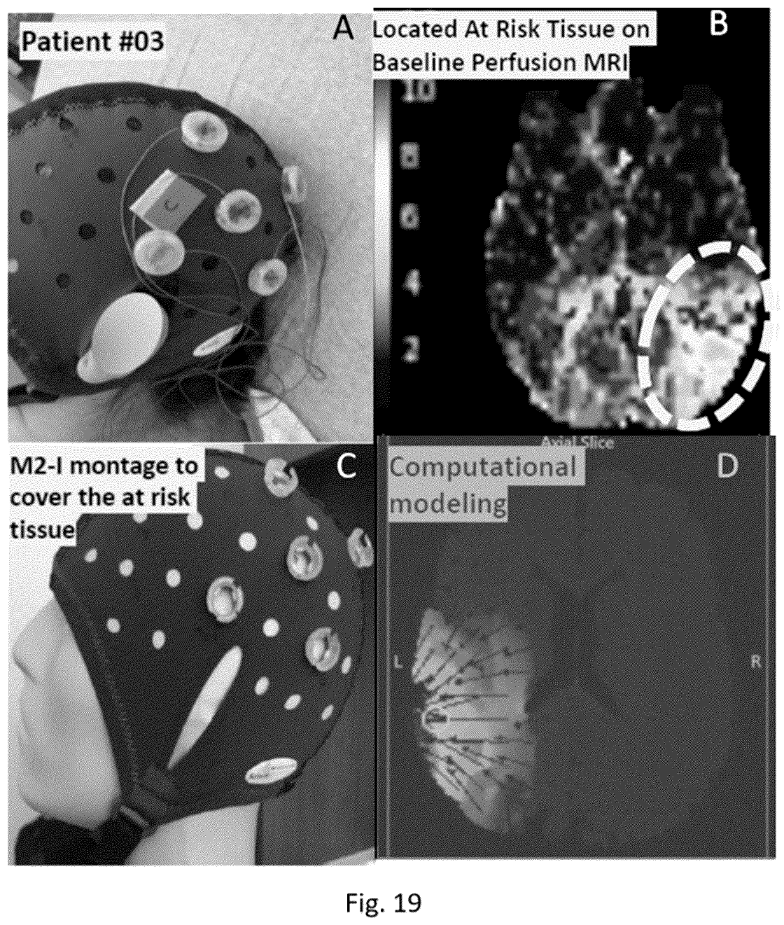

FIGS. 20A-20C depict changes in hypoperfusion/ischemic region, cerebral blood flow and volume of left posterior temporal region (circle) from baseline (first row) to 2 hours (2^{nd} row) to 24 hours (3^{rd} row). FIG. 20A depicts Tmax map (time takes for the contrast to pass through arterial, capillary and then venous system). Green region is indicative of a delay more than 6 sec in contrast passage due to left M3-M4 temporal branch occlusion indicative of hypoperfusion/ischemic region. There is evidence of resolution of the delay from baseline to 24 hr. FIG. 20B and FIG. 20C depicts relative cerebral blood flow (CBF) and cerebral blood volume (CBV) maps.

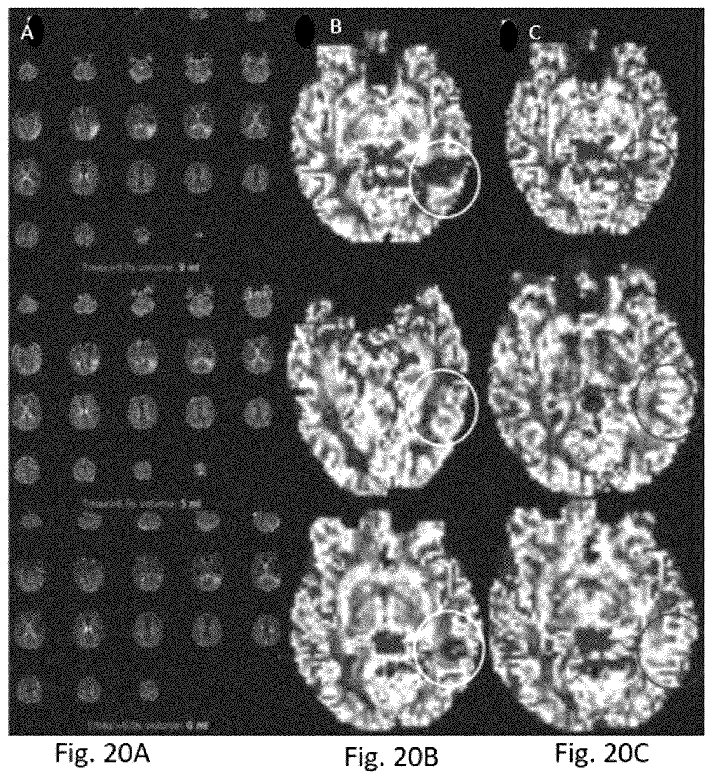

FIGS. 21A-21C depict diffusion weighted image (DWI), fluid attenuated inversion recovery (FLAIR), and gradient recall echo (GRE) MRI sequences at baseline (first row), 2-4 hours post-stimulation (second row), and 24-30 hours post-stimulation. Arrows indicate MCA slow flow which alleviated post-stimulation suggestive of recanalization. However, there was evidence of DWI region (infarct core) growth from baseline to 24 hours.

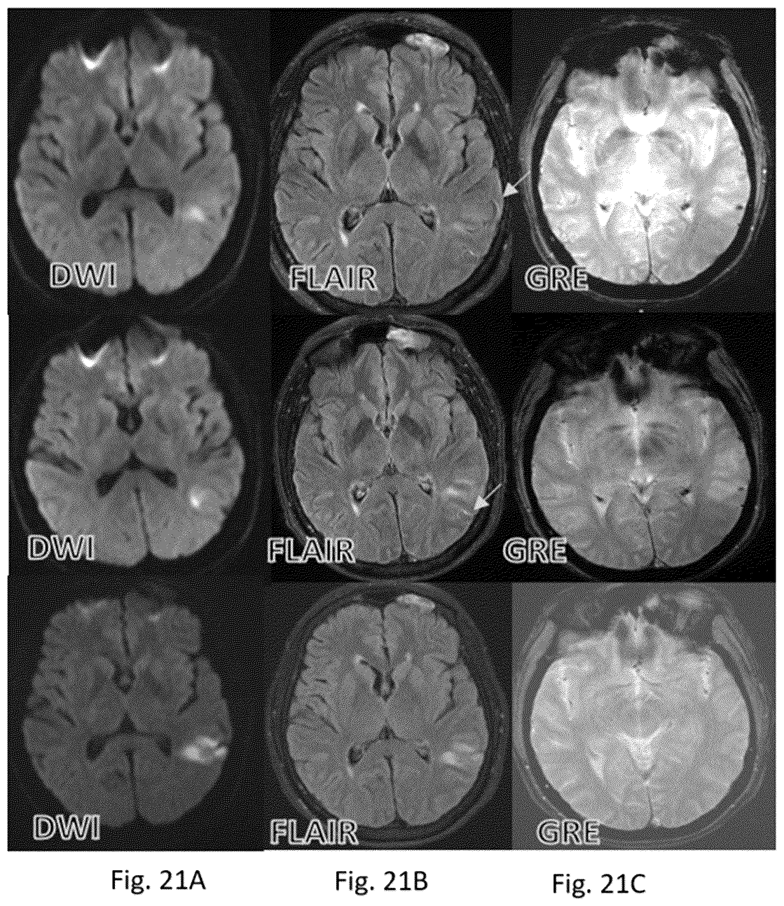

FIG. 22 depicts a 71-year-old female presented with a dense left homonymous hemianopsia and amnesia due to a right posterior cerebral artery (PCA) occlusion with persistent penumbra (circled ischemic tissue). She received 20 minutes of 1 mA of HD C-tDCS with PCA montage shown on the mannequin. The computational modeling shows the specificity of the coverage provided to the brain tissue at risk (circled) over the occipital region.

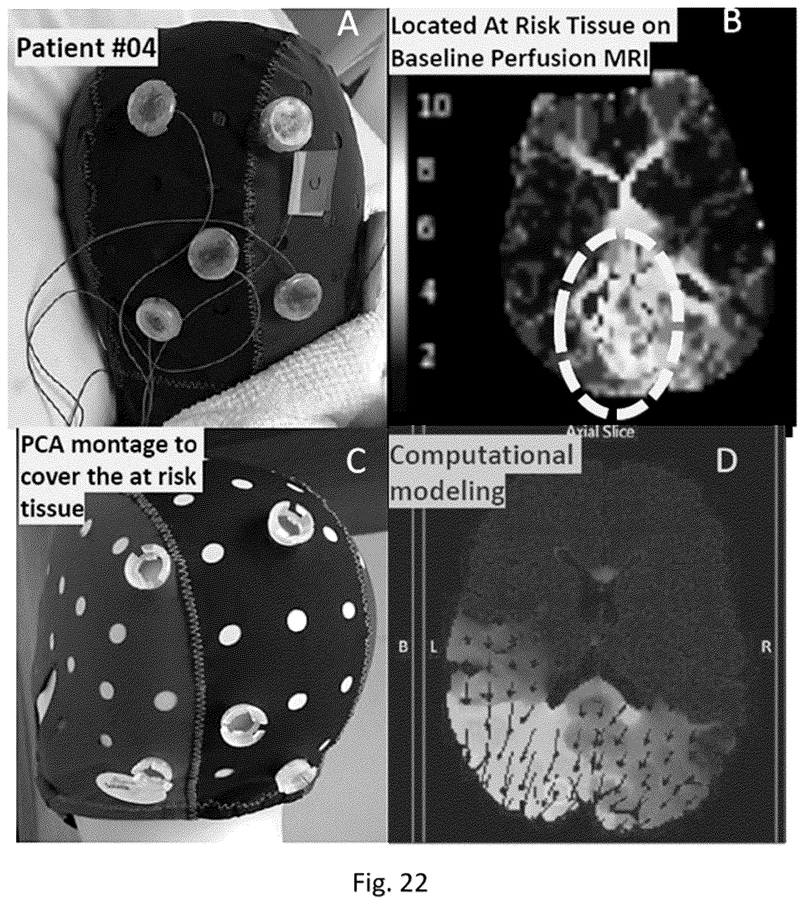

FIGS. 23A-23D depict a demonstration of HD C-tDCS effect on the occluded right PCA and cerebral blood flow of right occipital region (circled) from baseline (first row) to 2 hours (2^{nd} row) to 24 hours (3^{rd} row). FIG. 23A depicts MR angiography. Arrows suggest improvement of anterograde blood flow from baseline to 24 hr post stimulation indicative of recanalization of the occluded vessel. FIG. 23B depicts Tmax map (time takes for the contrast to pass through arterial, capillary and then venous system). Green region is indicative of a delay more than 6 sec in contrast passage due to vessel blockage indicative of the hypoperfusion/ischemic region. There is evidence of resolution of the delay from baseline to 24 hr. FIG. 23C and FIG. 23D depict relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is evidence of improvement in the CBF particularly at 24 hr shown in the circle.

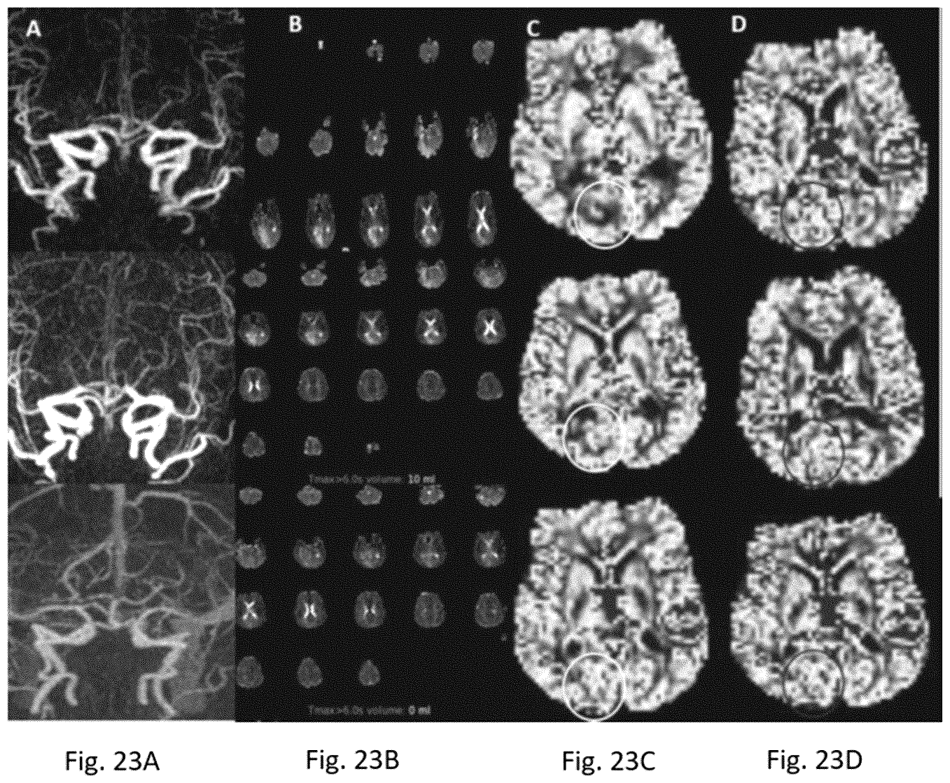

FIGS. 24A-24C depict diffusion weighted image (DWI), fluid attenuated inversion recovery (FLAIR), and gradient recall echo (GRE) MRI sequences at baseline (first row), 2-4 hours post-stimulation (second row), and 24-30 hours post-stimulation (third row). Arrow indicates PCA slow flow which alleviated post-stimulation suggestive of recanalization. The chevron arrow indicates CSF hyperintensity marker over the stimulated region.

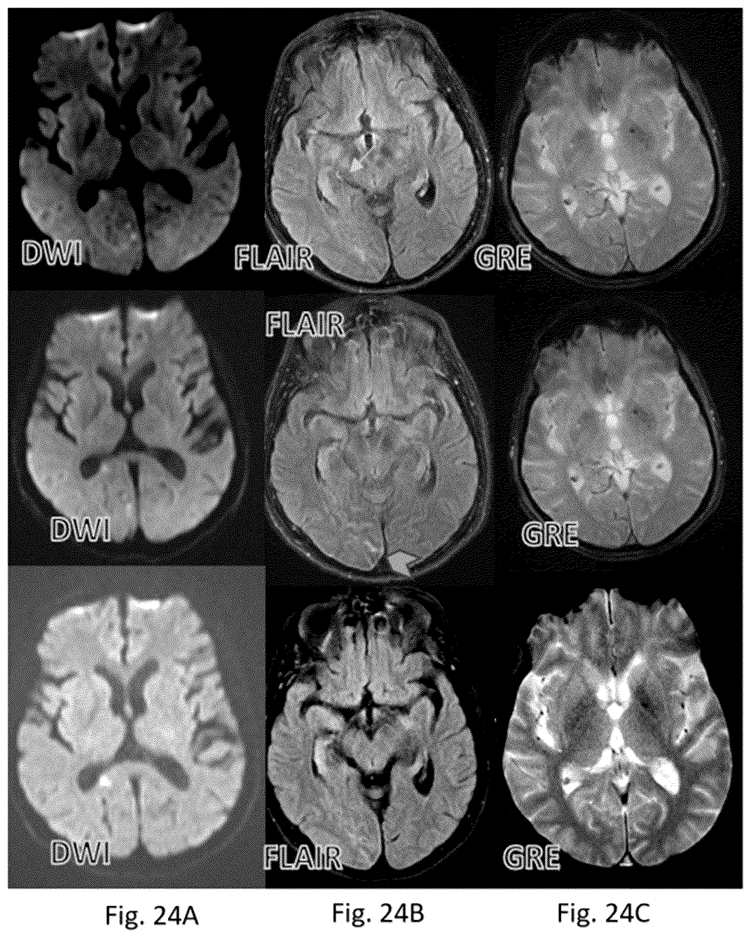

FIG. 25 depicts a 77-year-old female presented with left gaze deviation, global aphasia, right hemiparesis. She had occlusion of multiple M3-M4 branches including temporoparietal branch with persistent penumbra (circled ischemic tissue). She received 20 minutes of 2 mA of HD C-tDCS with MCA-M1 montage shown on the mannequin. The computational modeling shows the specificity of the coverage provided to the brain tissue at risk (circled).

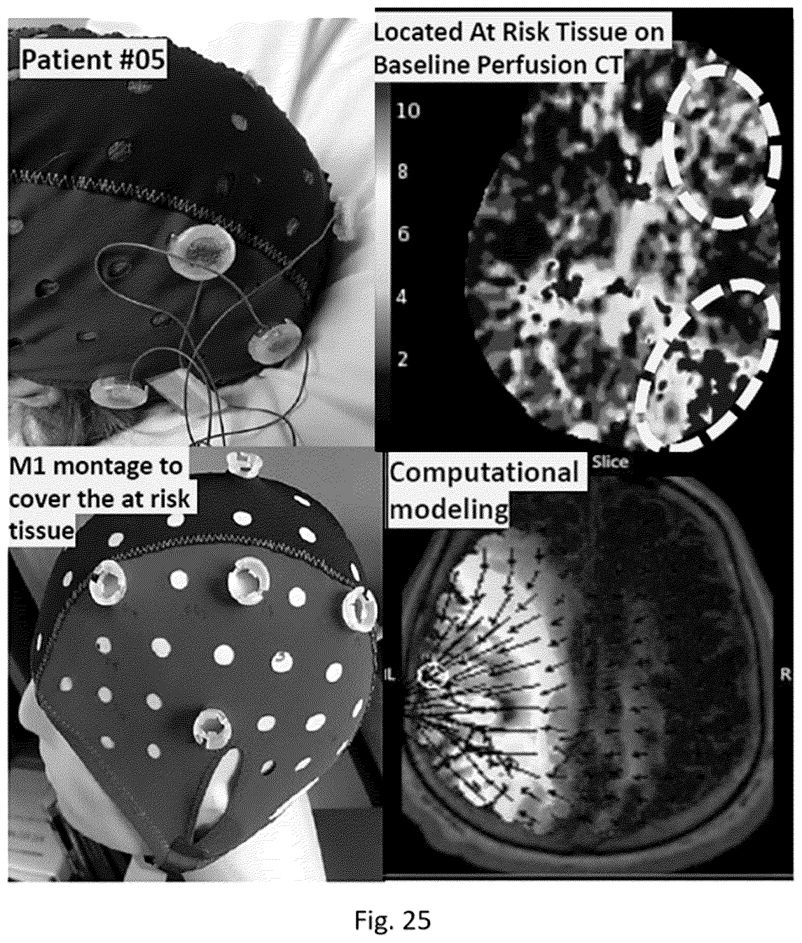

FIGS. 26A-26C depict a demonstration of HD C-tDCS effect on the occluded left M3-M4 temporal branch and cerebral blood flow of left posterior temporal region (circle) from baseline (first row) to 2 hours (2^{nd} row) to 24 hours (3^{rd} row). FIG. 26A depicts Tmax map (time takes for the contrast to pass through arterial, capillary and then venous system). Green region is indicative of the hypoperfusion/ischemic region with a delay more than 6 sec in contrast passage due to left M3-M4 temporal branch occlusion. There is evidence of resolution of the delay from baseline to 24 hr. FIG. 26B and FIG. 26C depict relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is evidence of improvement in the CBF particularly at 24 hr shown in the circle.

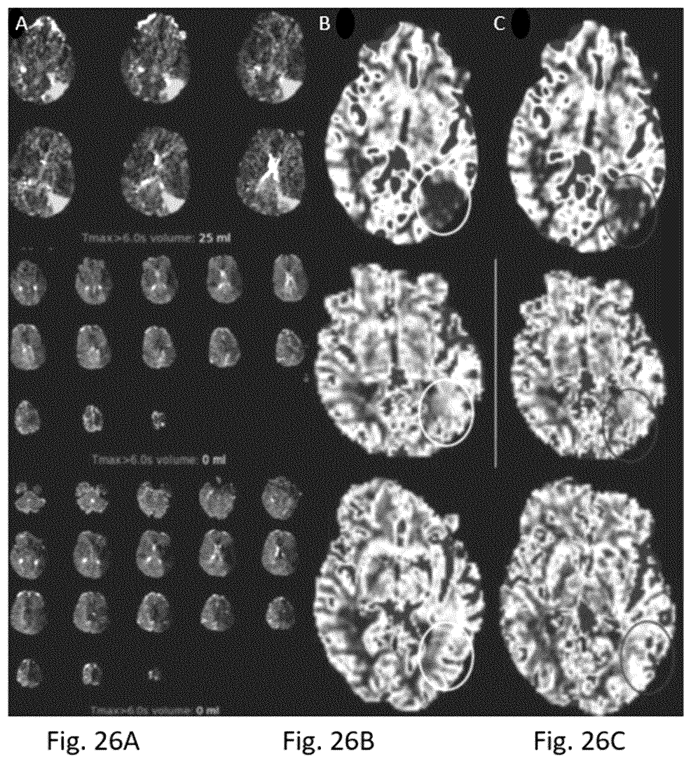

FIGS. 27A-27C depict CT head (first column, first row) and digital subtraction angiography (DSA) at baseline (third column, first row), restriction diffusion image (DWI), fluid attenuated inversion recovery (FLAIR), and gradient recall echo (GRE) MRI sequences at 2-4 hours post-stimulation (second row), and 24-30 hours post-stimulation (third row). Arrows indicate multiple distal MCA branch occlusions at baseline and chevron arrow indicates CSF hyperintensity marker over the stimulated region.

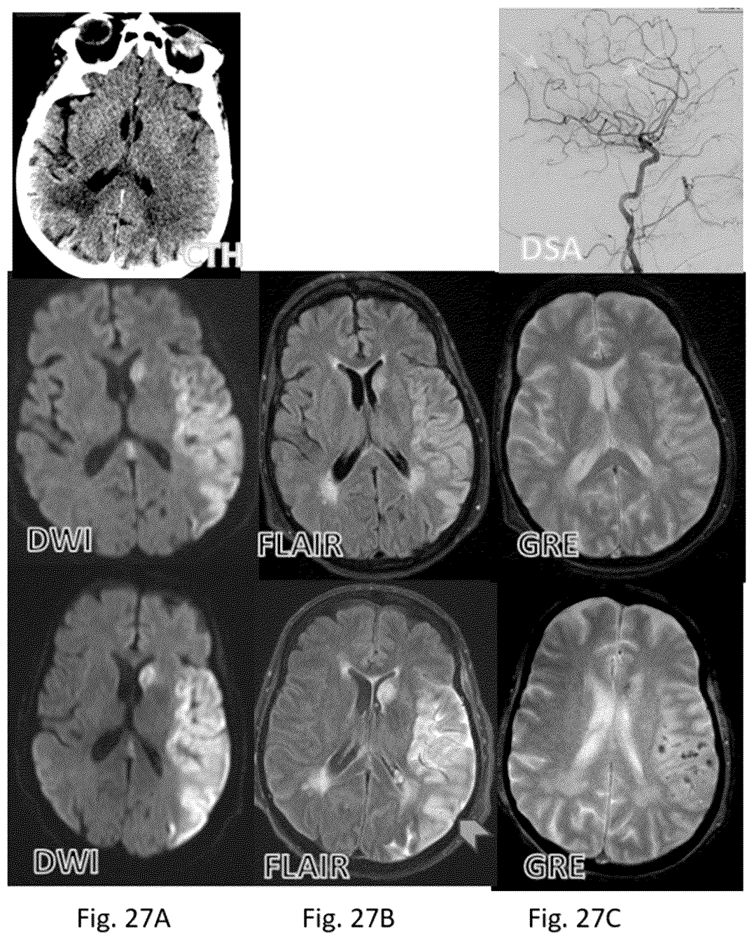

FIG. 28 depicts a 90-year-old male presented with expressive aphasia, a right homonymous hemianopsia, and left sided neglect due to a right inferior division middle cerebral artery (MCA) occlusion with persistent penumbra (circled ischemic tissue). He received 20 minutes of sham HD C-tDCS with MCA-M2I montage shown on the mannequin. The computational modeling shows the specificity of the coverage provided to the brain ischemic tissue (circled) over the inferior temporal region.

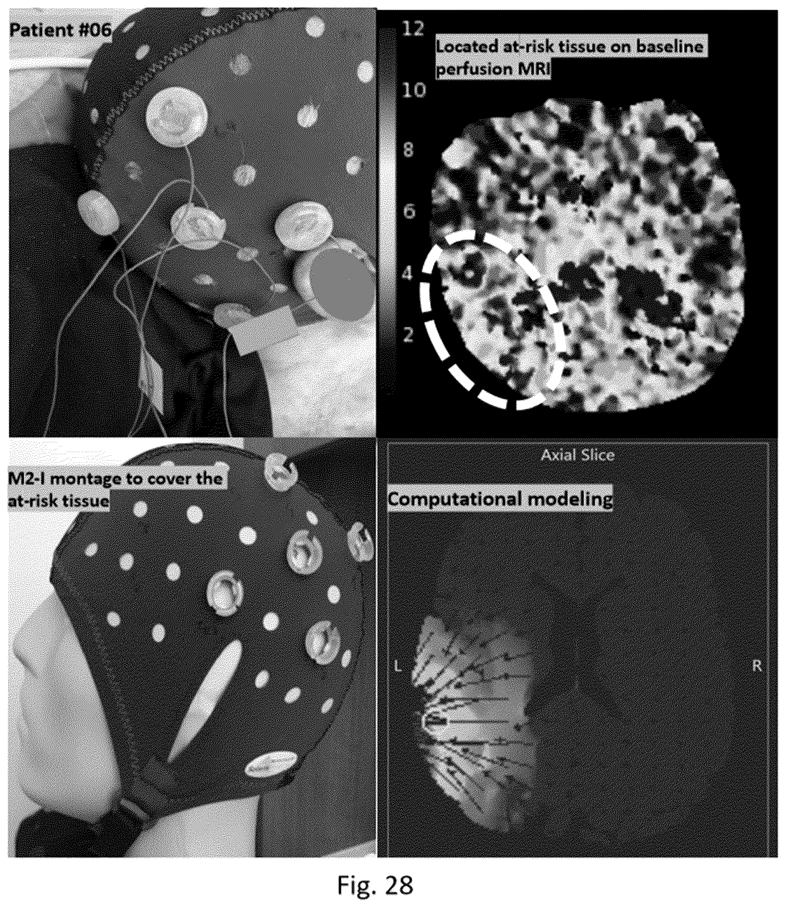

FIGS. 29A-29C depict a demonstration of Sham effect on the infarct core (first column), occluded right MCA-M2 (second column) and hypoperfusion of the right temporal region (third column) from baseline (first row) to 2-4 hours (2^{nd} row) and 24-30 hours (3^{rd} row). FIG. 29A depicts infarct core growth from baseline CT head to 2 hours and 24 hours DWI post-stimulation. FIG. 29B depicts CT angiography at baseline (first row) and MR angiography at 2-4 hours (2^{nd} row) and 24-30 hours (3^{rd} row). Arrows indicate persistent occlusion from baseline to 24 hr post stimulation. FIG. 29C depicts Tmax map (time takes for the contrast to pass through arterial, capillary and then venous system). Green region is indicative of a delay more than 6 sec in contrast passage due to vessel blockage indicative of the hypoperfusion/ischemic region. There is evidence of persistent delay from baseline to 24 hr.

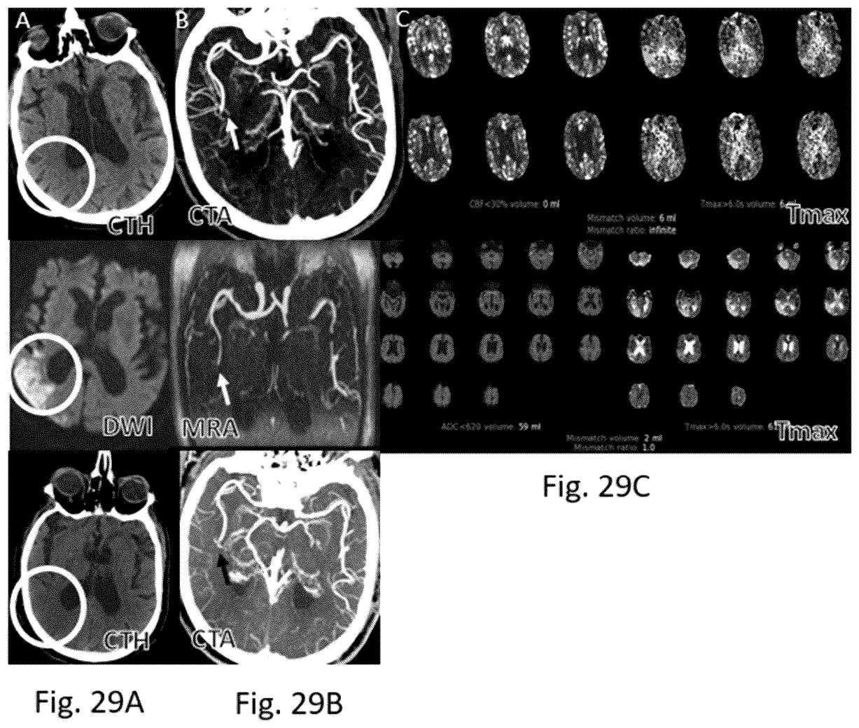

FIG. 30 depicts a 71-year-old female presented with a dense left homonymous hemianopsia, confusion, and amnesia due to a right posterior cerebral artery (PCA) occlusion with persistent penumbra (circled ischemic tissue). She received 20 minutes of 2 mA of HD C-tDCS with PCA montage shown on the mannequin. The computational modeling shows the specificity of the coverage provided to the brain ischemic tissue (circled) over the occipital region.

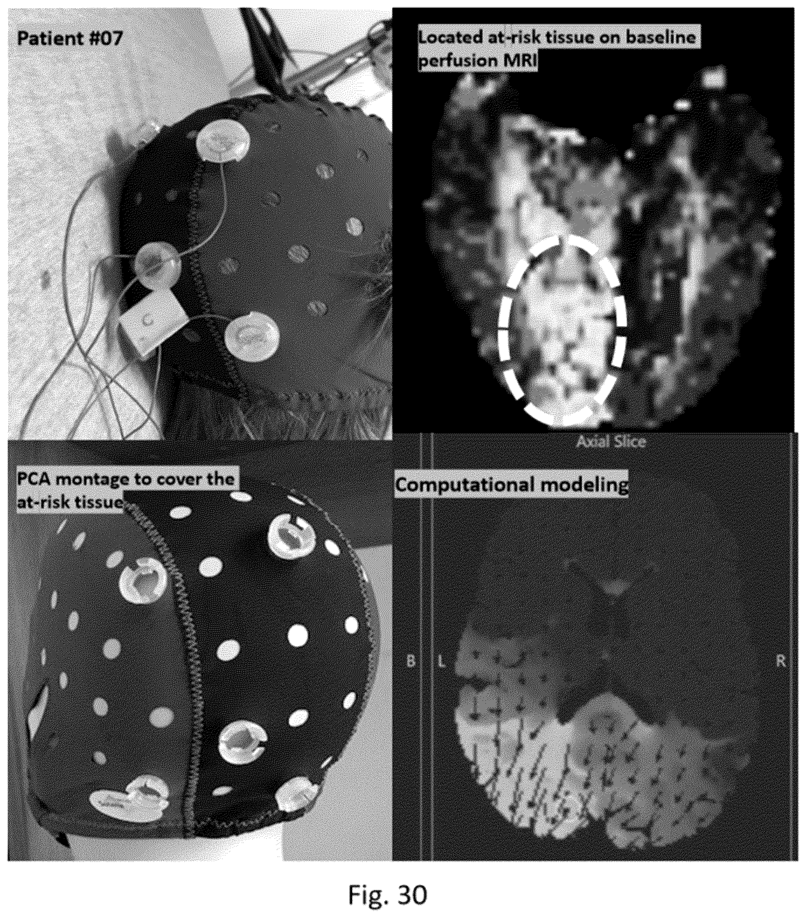

FIGS. 31A-31C depict a demonstration of HD C-tDCS effect on the hypoperfused left PCA territory and cerebral blood flow and volume of left occipital region (circle) from baseline (first row) to 2-4 hours (2^{nd} row) to 24-30 hours (3^{rd} row). FIG. 31A depicts Tmax map (time takes for the contrast to pass through arterial, capillary and then venous system). There is evidence of resolution of the hypoperfusion region (Tmax delay>6 sec) from baseline to 24 hr. FIG. 31B and FIG. 31C depict relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is evidence of improvement in the CBF particularly at 24 hr shown in the circle.

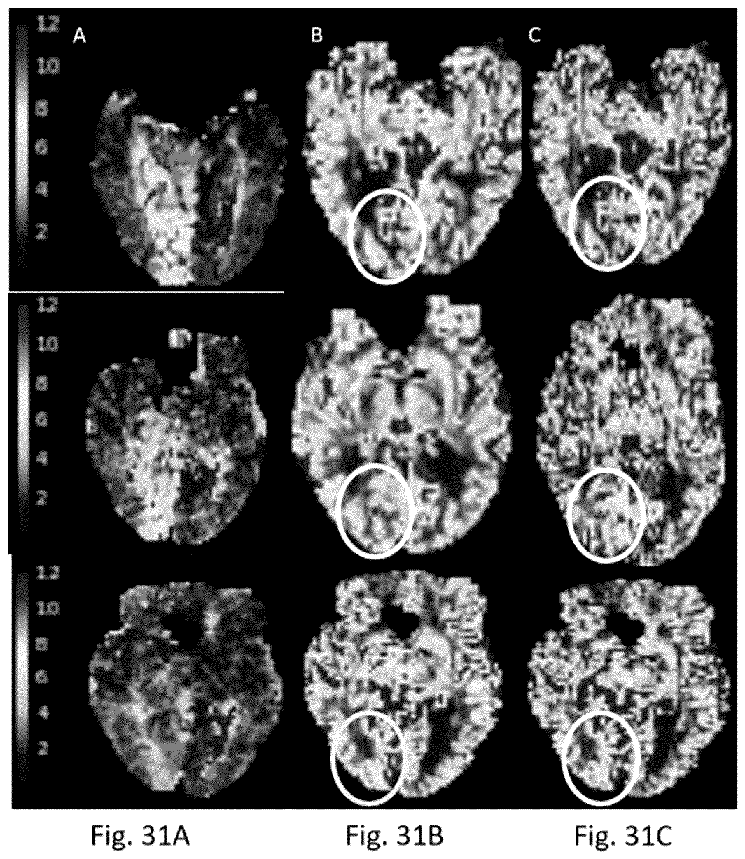

FIGS. 32A-32D depict diffusion weighted image (DWI), fluid attenuated inversion recovery (FLAIR), gradient recall echo (GRE) MRI sequences, and MR angiography, at baseline (first row), 2-4 hours post-stimulation (second row), and 24-30 hours post-stimulation. Arrows indicate right PCA occlusion at baseline which recanalized post-stimulation and chevron arrow indicates CSF hyperintensity marker over the stimulated region.

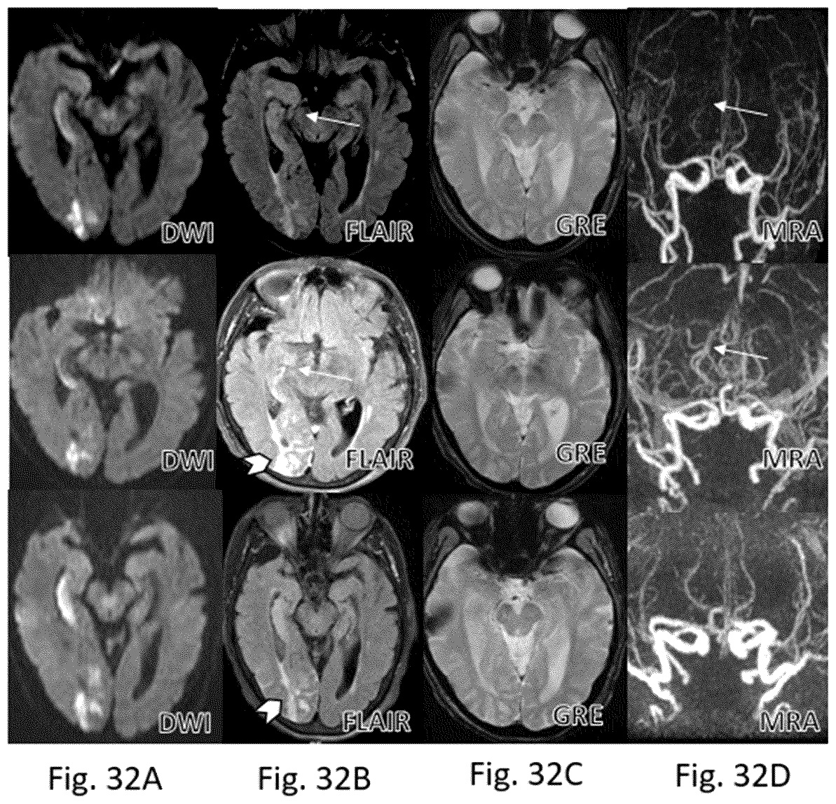

FIG. 33 depicts a 61-year-old male presented with right hemiparesis due to a left M3 branch (Rolandic branch) occlusion with persistent penumbra (circled ischemic tissue). He received 20 minutes of 2 mA of HD C-tDCS with MCA-S montage shown on the mannequin. The computational modeling shows the specificity of the coverage provided to the brain ischemic tissue (circled) over the occipital region.

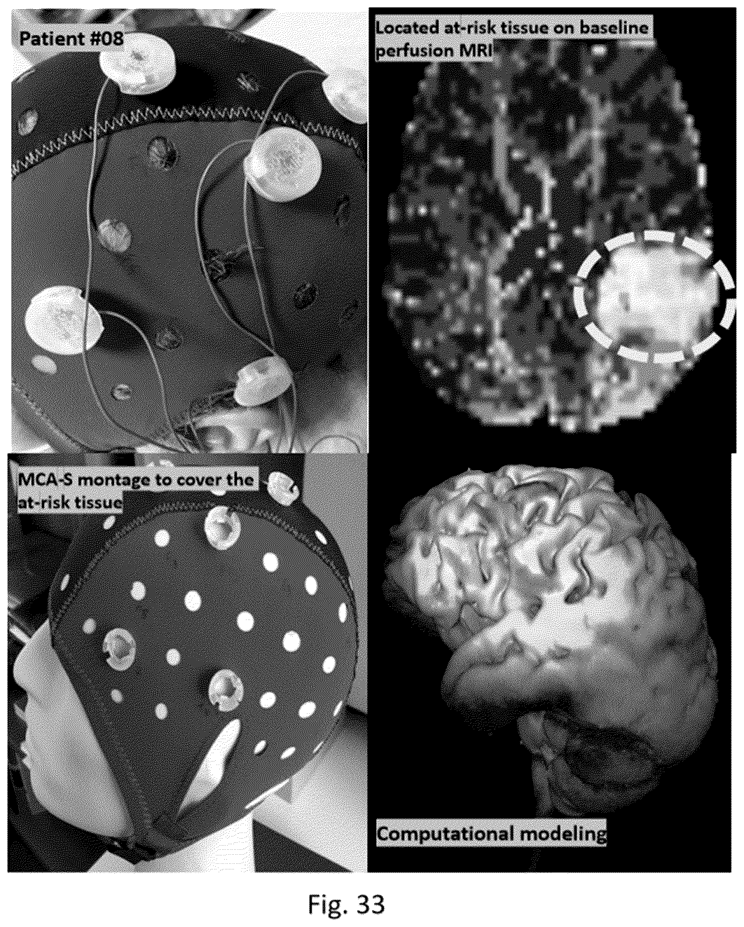

FIGS. 34A-34D depict a demonstration of HD C-tDCS effect on the hypoperfusion region due to occluded left M3-M4 temporal branch and cerebral blood flow ad volume of left posterior fronto-parietal region (circle) from baseline (first row) to 2 hours (2^{nd} row). FIG. 34A depicts MR angiography. FIG. 34B depicts Tmax map (time takes for the contrast to pass through arterial, capillary and then venous system). There is evidence of slight resolution of the hypoperfusion/ischemic region with Tmax delay more than 6 sec from baseline to 2 hours. FIG. 34C and FIG. 34D depict relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is evidence of improvement in the CBF particularly at 24 hr shown in the circle.

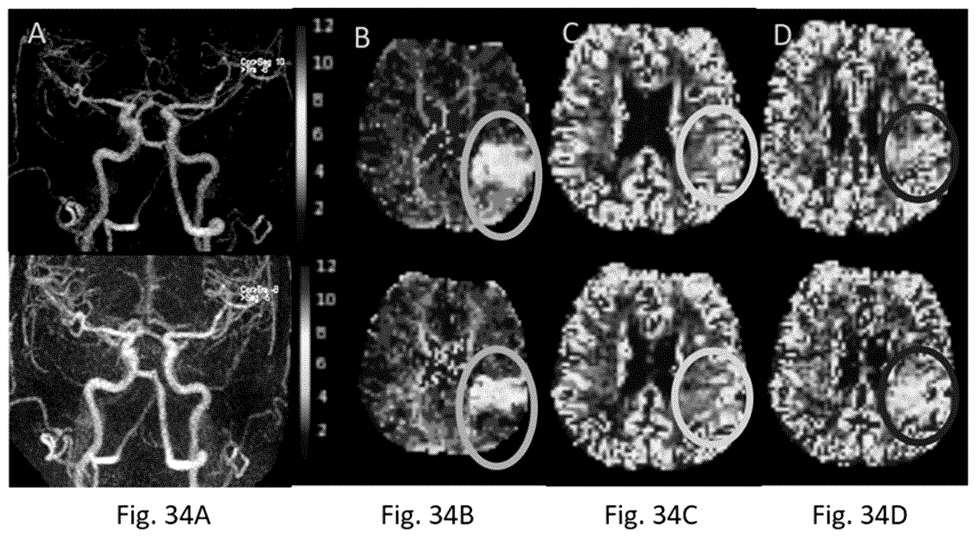

FIGS. 35A-35C depict diffusion weighted image (DWI), fluid attenuated inversion recovery (FLAIR), and gradient recall echo (GRE) MRI sequences at baseline (first row) and 2-4 hours post-stimulation (second row). Arrows indicate distal MCA branch (M3) occlusions at baseline and chevron arrows indicate CSF hyperintensity marker over the stimulated region.

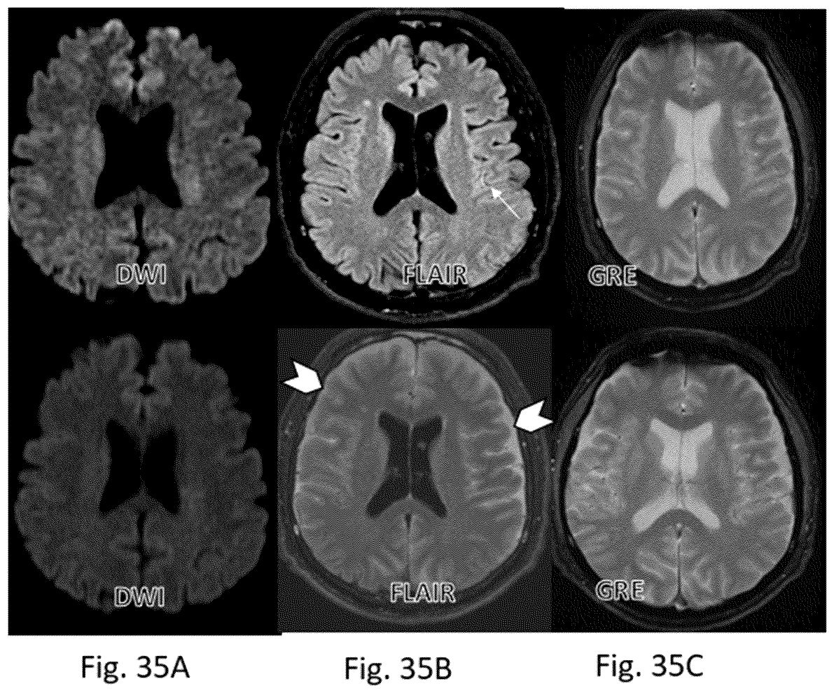

FIG. 36 depicts 89-year-old female presented with left sided neglect, confusion and agitation due to right M2 inferior branch occlusion with persistent penumbra (circled ischemic tissue). She received 20 minutes of 2 mA of HD C-tDCS with MCA-M2I montage shown on the mannequin. The computational modeling shows the specificity of the coverage provided to the ischemic brain tissue (circled) over the temporal region.

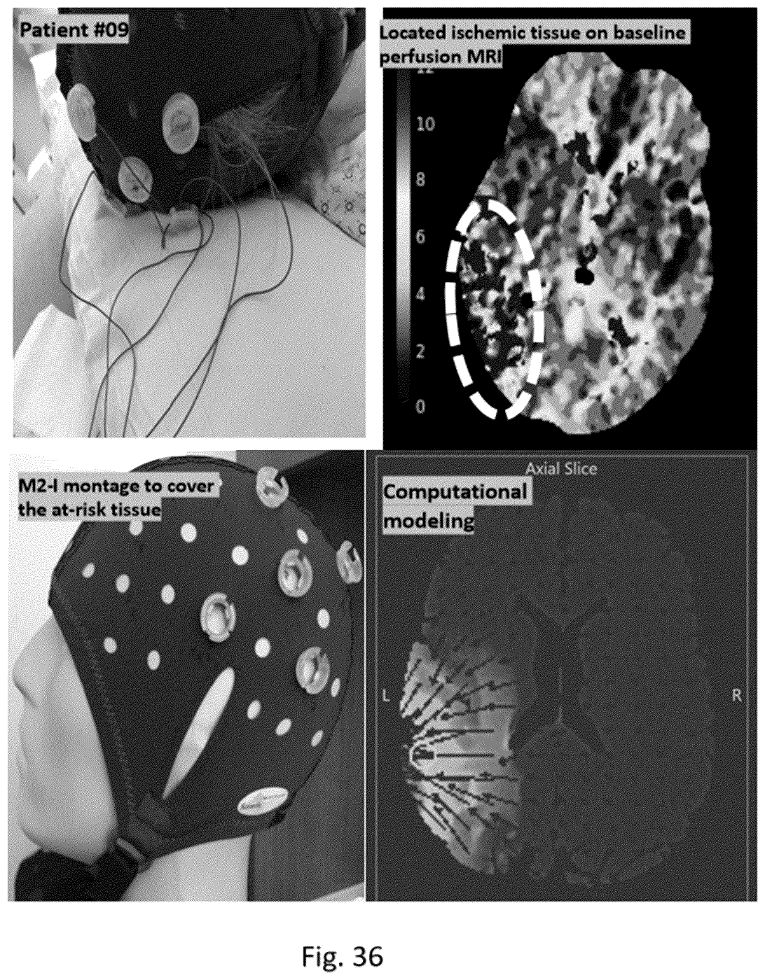

FIGS. 37A-37C depict a demonstration of HD C-tDCS effect on the hypoperfusion region due to occluded right M2 inferior branch and cerebral blood flow and volume of right posterior temporal region (circle) from baseline (first row) to 2 hours (2^{nd} row), and 24 hours (3^{rd} row). FIG. 37A depicts Tmax map (time takes for the contrast to pass through arterial, capillary and then venous system). There is evidence of slight resolution of the hypoperfusion/ischemic region with Tmax delay more than 6 sec from baseline to 2 hours and 24 hours post-stimulation. FIG. 37B and FIG. 37C depict relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is evidence of improvement in the CBF and CBV.

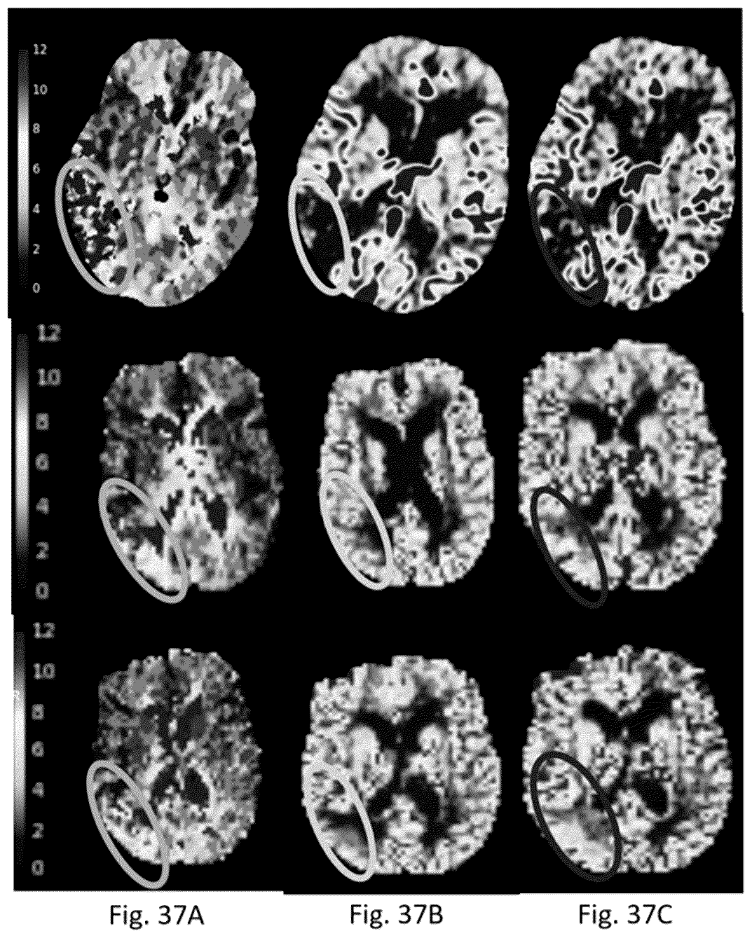

FIGS. 38A-38D depict CT head at baseline (first column, first row), diffusion weighted image (DWI), fluid attenuated inversion recovery (FLAIR), and gradient recall echo (GRE) MRI sequences at 2-4 hours (second row) and 24-30 hours post-stimulation (third row). The last column depicts CT angiography at baseline (1^{st} row) and MR angiography at 2-4 hours (second row) and 24-30 hours post-stimulation (third row). Arrows indicate the right MCA distal occlusion with no recanalization at 2 and 24-hour post stimulation.

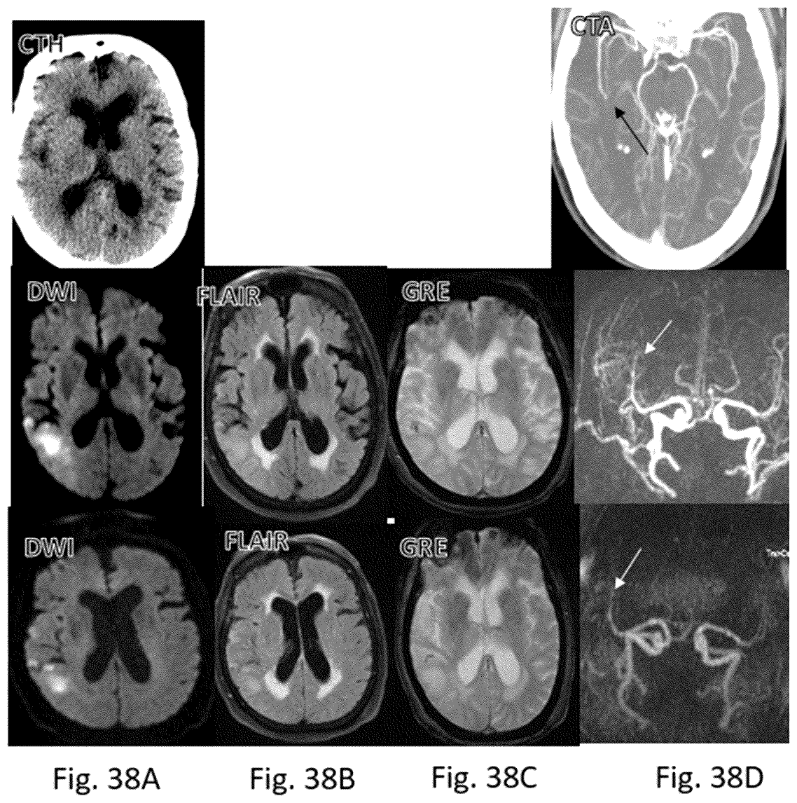

FIG. 39 depicts a 73-year-old male presented with left hemiparesis and neglect due to a right M3-M4 branch occlusion with persistent penumbra (circled ischemic tissue). He received 20 minutes of SHAM with MCA-S montage shown on the mannequin. The computational modeling shows the specificity of the coverage provided to the brain ischemic tissue (circled) over the occipital region.

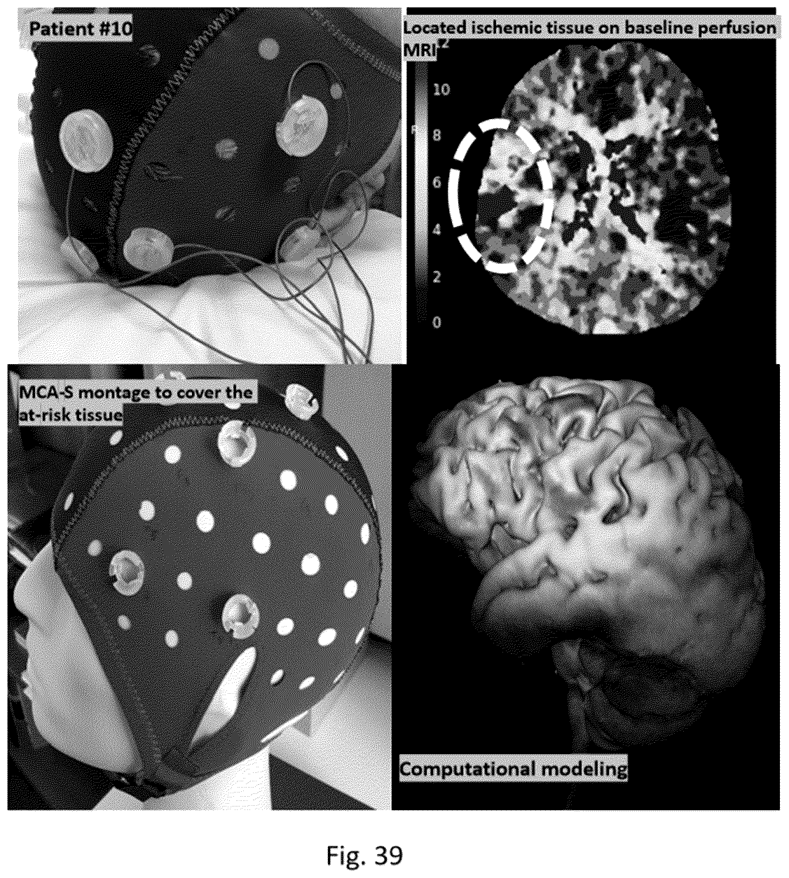

FIGS. 40A-40C depict a demonstration of Sham effect on the hypoperfusion region due to occluded right M3-M4 branch and cerebral blood flow and volume of left anterior fronto-parietal region (circle) from baseline (first row) to 2-4 hours (2^{nd} row) and 24-30 hours (3^{rd} row). FIG. 40A depicts Tmax map (time takes for the contrast to pass through arterial, capillary and then venous system). There is evidence of slight resolution of the hypoperfusion/ischemic region with Tmax delay more than 6 sec from baseline to 2-4 hours (2^{nd} row) and 24-30 hours (3^{rd} row). FIG. 40B and FIG. 40C depict relative cerebral blood flow (CBF) and cerebral blood volume (CBV) maps. Blue color indicates low CBF/CBV and yellow-orange indicates increased CBF/CBV comparing the baseline map to 2 hr and then 24 hr, there is no evidence of improvement in the CBF or CBV.

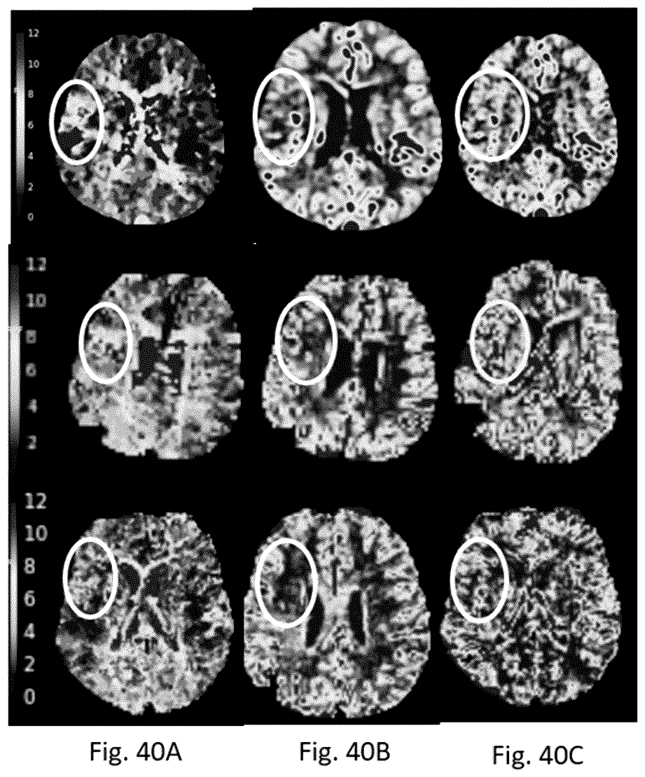

FIGS. 41A-41D depict CT head at baseline (first column, first row), diffusion weighted image (DWI), fluid attenuated inversion recovery (FLAIR), and gradient recall echo (GRE) MRI sequences at 2-4 hours (second row) and 24-30 hours post-stimulation (third row). The last column depicts CT angiography at baseline (1^{st} row) and MR angiography at 2-4 hours (second row) and 24-30 hours post-stimulation (third row). Arrows indicate the right MCA distal occlusion with no recanalization at 2 and 24-hour post stimulation.

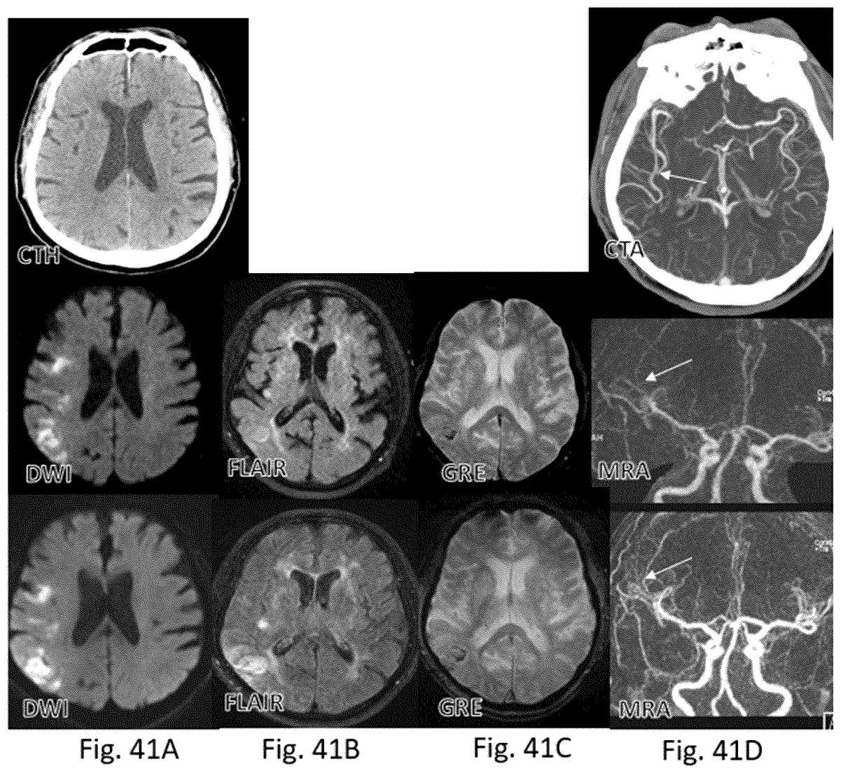

FIG. 42 depicts illustration of electrical field, in coronal, sagittal, axial, and external views resulted from F3, Cz, P3, T7, C3 montage, concentrated over the middle cerebral artery (MCA-M1) territory.

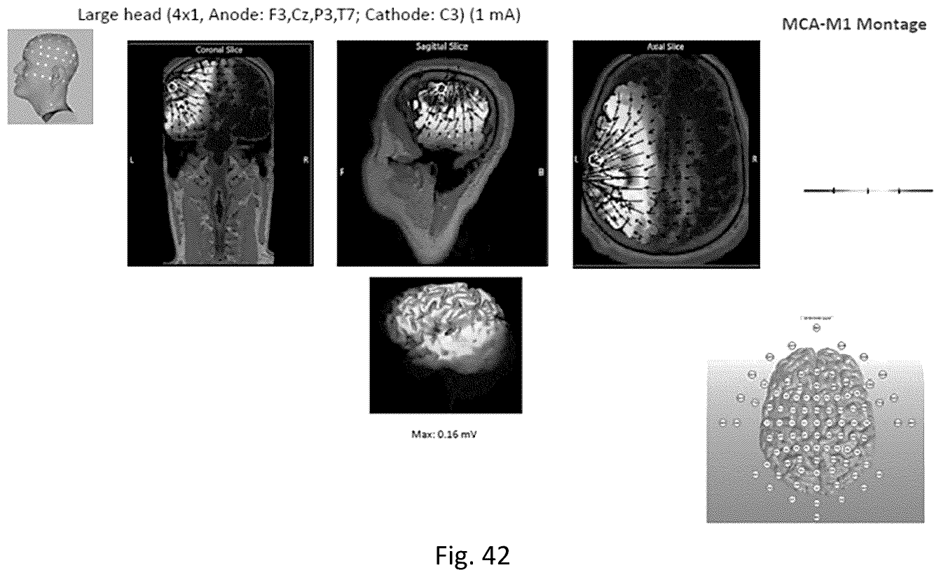

FIG. 43 depicts illustration of electrical field, in coronal, sagittal, axial, and external views resulted from C1, F1, F7, T7, FC3 montage, concentrated over the superior branch of middle cerebral artery (MCA-M2S) territory.

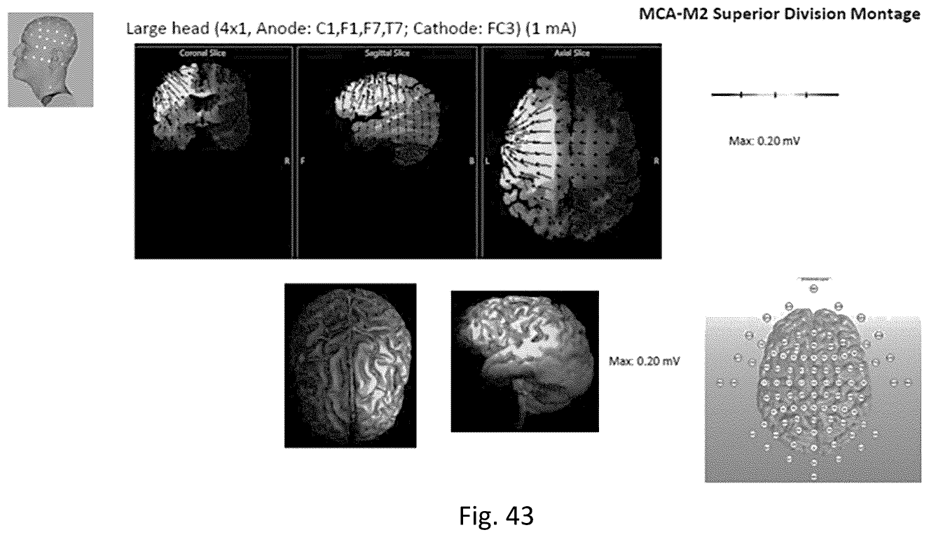

FIG. 44 depicts illustration of electrical field, in coronal, sagittal, axial, and external views resulted from C3, P3, T7, P7, CPS montage, concentrated over the inferior branch of middle cerebral artery (MCA-M2I) territory.

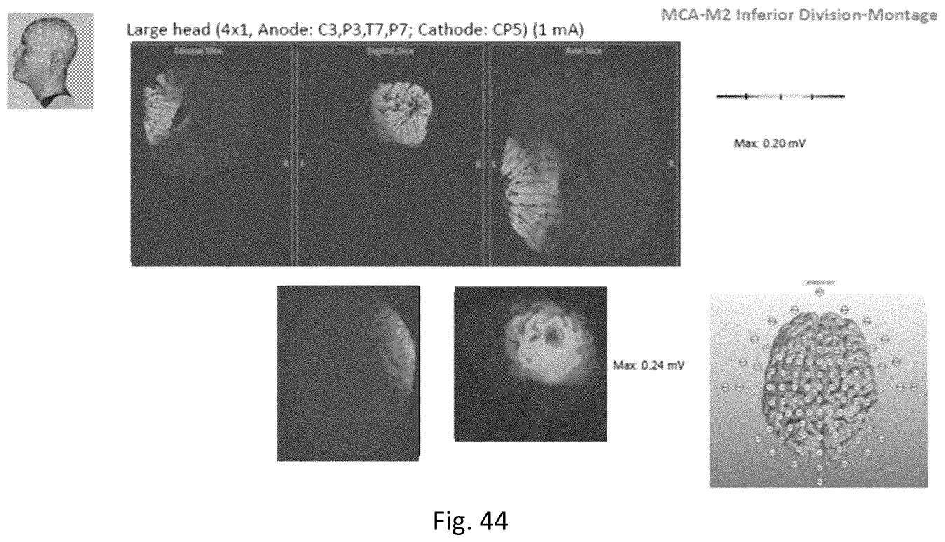

FIG. 45 depicts illustration of electrical field, in coronal, sagittal, axial, and external views resulted from FP2, FC4, FPz, FCz, F2 montage, concentrated over the anterior cerebral artery (ACA) territory.

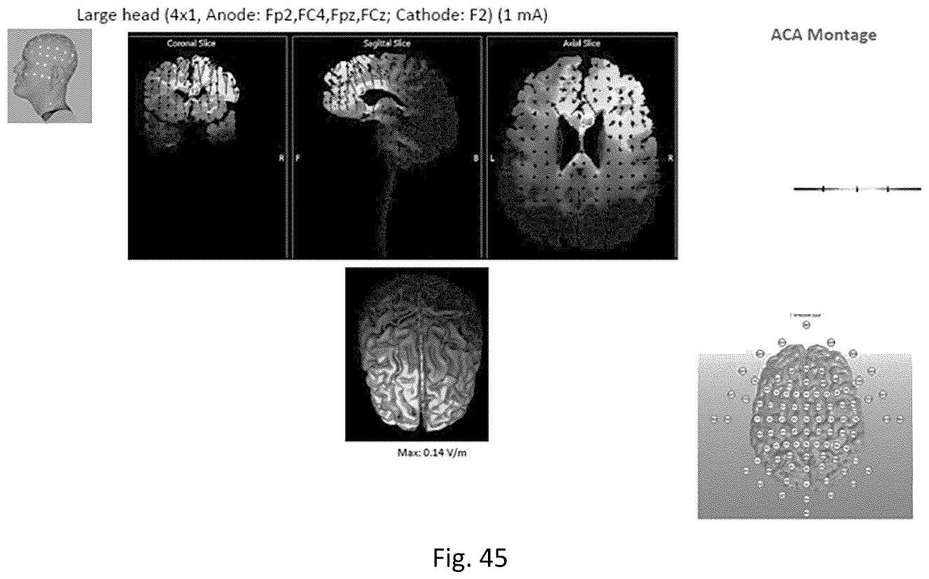

FIG. 46 depicts illustration of electrical field, in coronal, sagittal, axial, and external views resulted from P3, PO9, Pz, Iz, O1 montage, concentrated over the superior branch of posterior cerebral artery (PCA) territory.

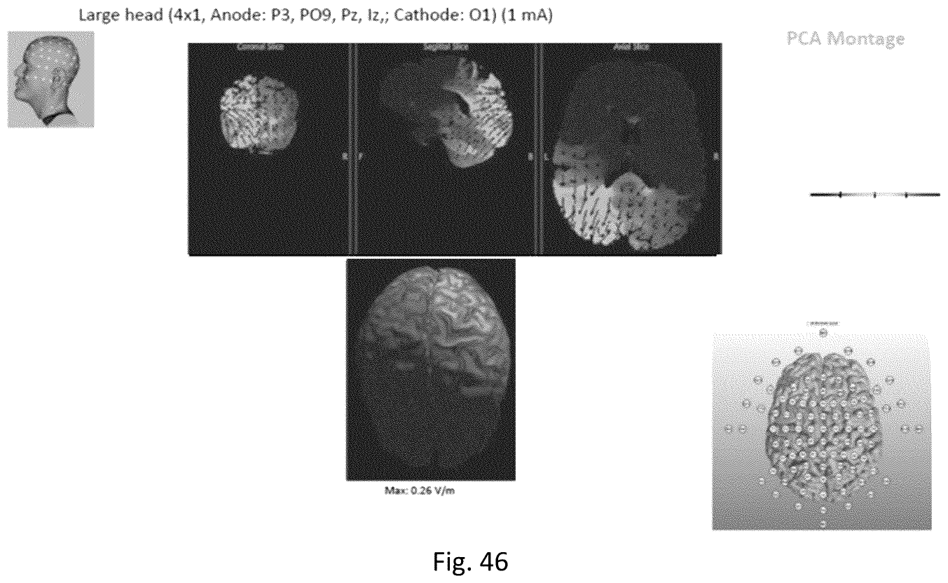

FIG. 47 depicts illustration of electrical field, in coronal, sagittal, axial, and external views resulted from O1, EX1, EXS, P7, PO9 montage, concentrated over the posterior inferior cerebellar artery (PICA) territory.

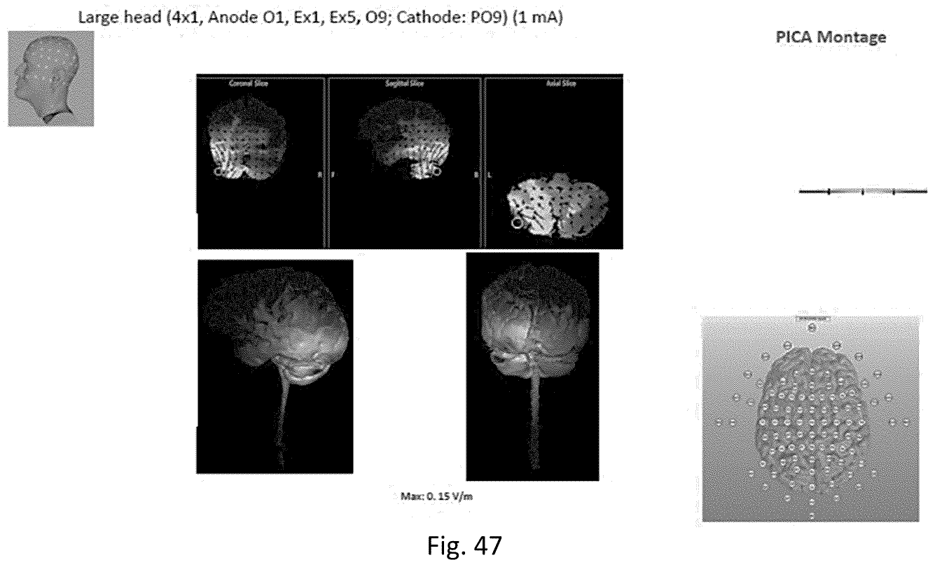

FIG. 48 depicts illustration of electrical field, in coronal, sagittal, axial, and external views resulted from F3, Fpz,Cz,CP5,F9 montage, concentrated over the internal carotid artery (ICA) territory.

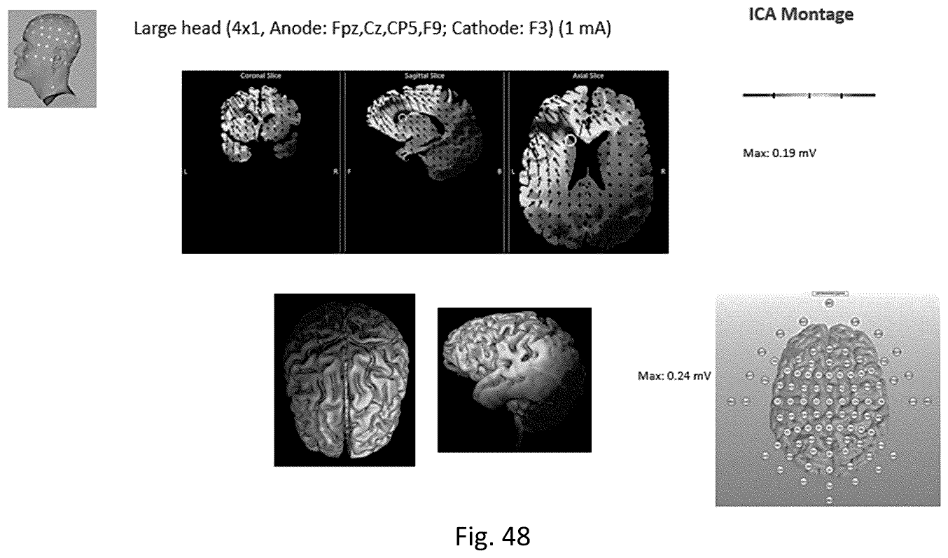

FIG. 49 depicts a study flow diagram.

FIG. 50 depicts scans of an exemplary patient.

FIG. 51 depicts scans of an exemplary patient.

FIG. 52 depicts exemplary results.

### DETAILED DESCRIPTION

It is to be understood that the figures and descriptions of the present invention have been simplified to illustrate elements that are relevant for a clear understanding of the present invention, while eliminating, for the purpose of clarity many other elements found in the field of transcranial direct current stimulation. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. The patient, subject or individual may be a mammal, and in some instances, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Electrical Stimulation System and Device

The present invention provides a device and system for non-invasively providing targeted individualized treatment for acute ischemic stroke. The system of the present invention is intended to provide an electrical current, transcranially, to a specific area of the subject's brain undergoing acute ischemic stroke. In one embodiment, the present invention is intended to provide a low-dose direct electrical current, transcranially in acute stroke patients. In one embodiment, the treatment may be delivered in acute stroke due to a large or medium/distal cortical vessel occlusion. In another embodiment, the treatment may be delivered in acute stroke due to penetrating vessel occlusion. The present invention aims to reduce the extent of brain injury caused by acute stroke and overall improve the quality of life of subjects suffering from acute ischemic stroke. Furthermore, the present invention aims to enhance the effectiveness of reperfusion therapies as an adjunct treatment or to reduce the deleterious effect of reperfusion injury.

As described above, given promising results in acute stroke models a dose-escalation feasibility and tolerability trial called TESSERACT wase performed. TESSERACT (Transcranial Electrical Stimulation EaRly After onset Clinical Trial) developed and tested High-Definition Cathodal Transcranial Direct Current Stimulation (HD C-tDCS) as a novel targeted treatment for acute ischemic stroke. In this pilot study, the electrical current was delivered only to the ischemic tissue via individualized high-definition electrode montages. HD C-tDCS was efficiently applied in emergency settings and was well-tolerated. Furthermore, the hypoperfused/ischemic region volume was reduced in the active groups by median 100% (46% to 100%) vs increased by 325% (112% to 412%) in sham. Penumbral salvage was attained in the active groups, median 66% (IQR 29%-80.5%), and not in the sham, in sham. Collaterals were enhanced in the active groups, with quantitative relative cerebral blood volume (qrCBV) increased in active groups, median 64% (40% to 110%), and not in sham patients, median −4% (−7% to 1%). In addition, for the imaging biomarker of collateral enhancement (qrCBV), the response was consistent with a dose-response effect, highest at Tier 2, intermediate at Tier 1, and lowest at sham. Directionally favorable effects were seen for improved perfusion and salvage of threatened tissue. Lastly, higher rates of recanalization (80%) was observed in active patients compared to sham (30%).

TESSERACT was the first-in-human study of HD tDCS as individualized cytoprotection and collateral enhancing strategy (Neurovascular Modulation) in acute ischemic stroke patients. As a single-center, proof-of-concept study, it successfully enabled the development of techniques for rapid initiation of stimulation. In addition, it provided preliminary evidence of favorable effects of HD C-tDCS upon perfusion, collateral enhancement, and penumbral salvage. Collateral enhancement likely also contributed to higher vessel recanalization rates, as collateral augmentation has been shown to increase delivery of endogenous thrombolytic both to the proximal and distal ends of the clot.

The trial thereby laid the foundation for TESSERACT 2; a multi-site phase 2a adaptive dose-optimization study to identify an HD C-tDCS dose regimen showing adequate safety and substantial efficacy on imaging biomarkers of penumbral salvage to advance to future, definitive, pivotal phase 2b/3 trials.

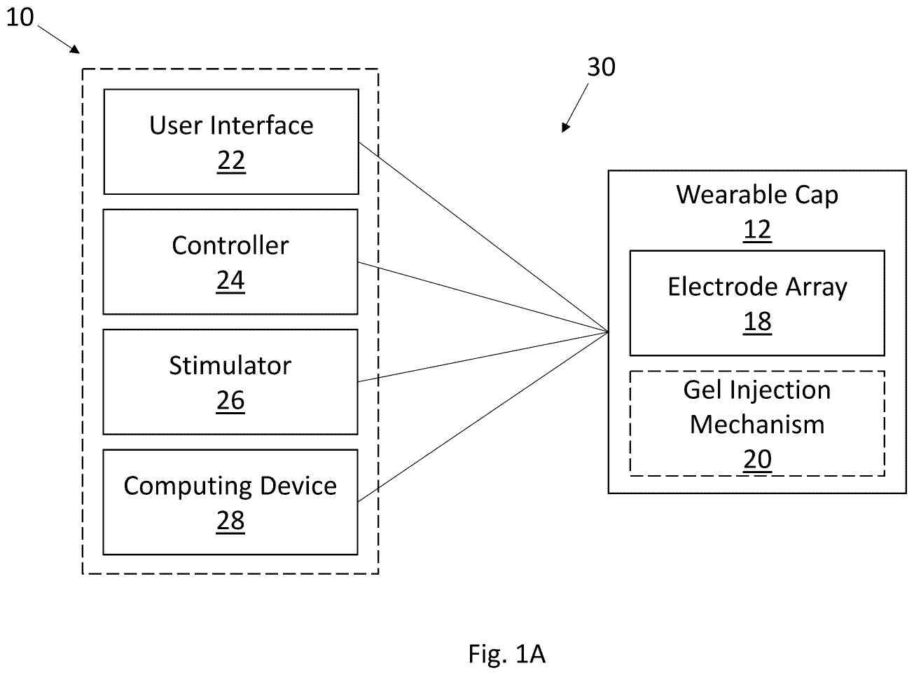

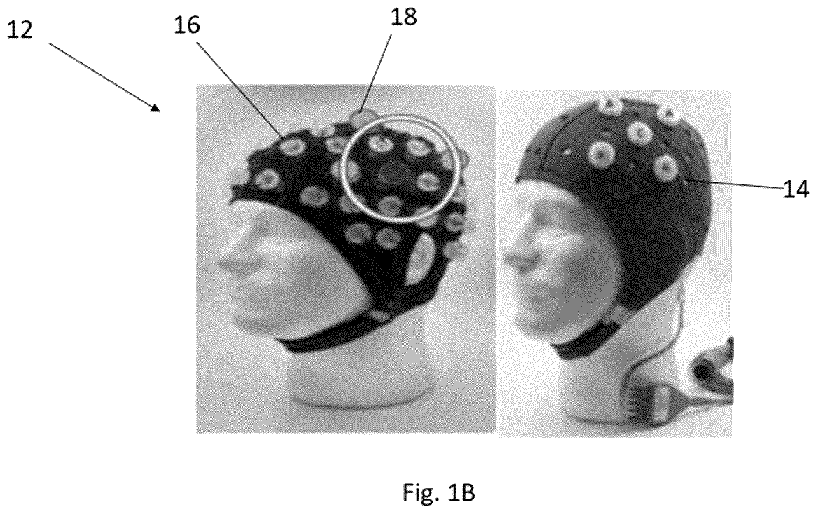

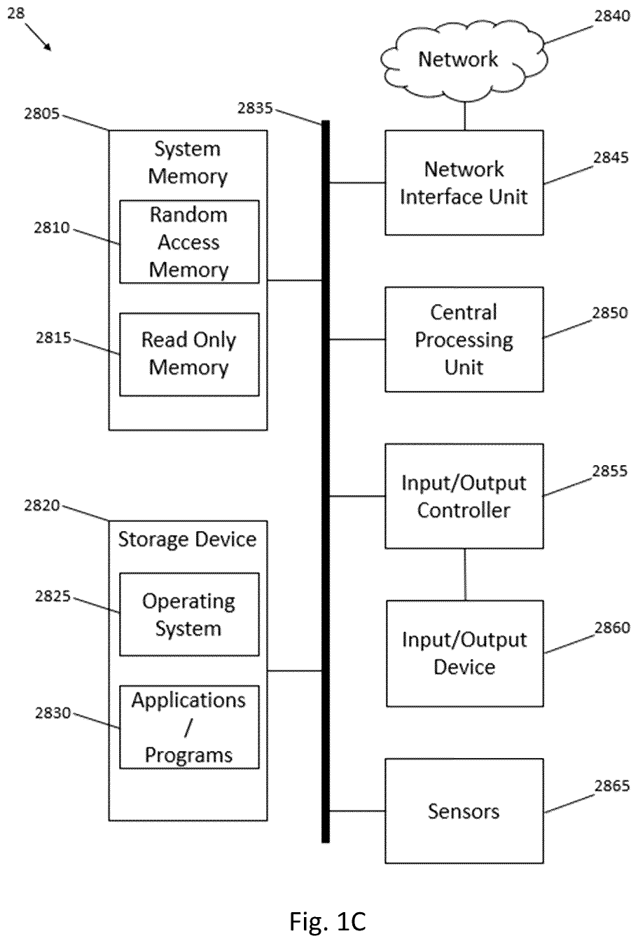

Referring now to FIGS. 1A-1B, an electrical stimulation system 10 and device 12 of the present invention is shown. In some embodiments, the system 10 comprises a cap having a plurality of openings 14, a plurality of electrode holders 16, a plurality of electrodes 18, and optionally a gel injection mechanism 20. In some embodiments the system 10 further comprises a control panel or user interface 22, controller 24, a current generator or stimulator 26, and/or a computing device 28, connected via wired or wireless communication and/or electrical means 30 to the wearable cap 12, electrode array 18, and/or gel injection mechanism 20. In some embodiments, the connection mean 30 comprises a plurality of cables, a single cable, or suitable wireless communication protocol including but not limited to Bluetooth, near-field communications (NFC), or wireless internet protocols.

In some embodiments, one or more of the control panel or user interface 22, controller 24, current generator or stimulator 26, and/or computing device 28 are integrated into a single unit to reduce the overall size of the system 10 an enhance portability of the system 10.

In some embodiments, cap 12 comprises plurality of a plurality of electrodes in an array 18 affixed to, embedded or otherwise integrated into a plurality of openings 14 on the cap. In one embodiment, the distance between two openings is according to the 10-10 international system conventionally used in EEG. Cap 12 may be constructed of any material known to those skilled in the art. In one embodiment, cap 12 is designed and constructed to be durable, resilient, flexible, and easy to clean. Cap 12 may be secured about the subject's head by means commonly known to those in the art, including, but not limited to, a garment completely encompassing the subject's head, a strap that is secured by compression or elastic means, or may utilize common fastening methods such as hook-and-loop, belt-type, snap connectors, or the like. In one embodiment, cap 12 is custom molded that fits snugly but comfortably about the subject's head, and is capable of maintaining a secure placement with minimal shifting, drift, or other movement of device 10, for the entire length of time necessary for stimulation. In one embodiment, an adhesive layer is capable of providing a secure, stable attachment to the subject's head in the presence of dirt, sweat, and other detritus which may be covering the subject's skin during application without the need for washing, cleaning or otherwise preparing the area of application.

The plurality of electrodes 18 are affixed to, embedded in, or otherwise integrated into plurality of electrode holders 16 that is attached to plurality of openings 14. This allows keeping plurality of electrodes 18 in a stable arrangement or array with respect to each other. In one embodiment, plurality of electrode holders 16 may be made from plastic. In one embodiment, plurality of electrode holders 16 may be made from any material known to one skilled in the art. In one embodiment, plurality of electrode holders 16 have a locking mechanism that locks plurality of electrodes 18 in place. The locking mechanism can be any mechanism known to one skilled in the art. In one embodiment, cap 12 and plurality of electrodes 18 can be sterilized between each use.

In one embodiment, plurality of electrodes 18 may be any of those commonly known in the art of tDCS. In one embodiment, plurality of electrodes 18 have a surface area of 1 cm². In one embodiment, plurality of electrodes 18 require conductance gel. In one embodiment, conductance gel may be loaded into plurality of electrode holders 16 before the plurality of electrodes 18 are mounted. In one embodiment, plurality of electrodes 18 require the application of a conductive gel or paste. Therefore, plurality of electrodes 18 may have any necessary conductive fluids pre-applied. In one embodiment, plurality of electrodes 18 are dry physiological electrodes requiring no conductive fluid at all. In one embodiment, system 10 may include an automatic mechanism to inject gel into plurality of electrode holders 16 to improve the speed of the system implementation without jeopardizing the quality of the electrical conductance.

Prior to stimulation, in some embodiments, the plurality of electrodes 18 are monitored for any sign of damage such as chipping. In one embodiment, plurality of electrodes 18 are discarded after being exposed to at least 5 to 10 cycles of stimulation.

In one embodiment, the plurality of electrodes 18 may be arranged in an array. In one embodiment, plurality of electrodes 18 are arranged in a 4×1 array. By this, it is meant that there is at least one central cathode electrode surrounded by at least four return electrodes arranged in a ring around the center electrode. The electrical current flows into the subject's brain from the 4 reference electrodes and flows out from the center cathode electrode'”. In one embodiment, the electrodes are configured and placed in a montage, to apply the electrical current to target a particular portion of the subject's brain (target ischemic tissue).

The target tissue can be defined as the area in the patient's head or brain that is being targeted for treatment. In one embodiment, a “target tissue” is a specific one or more tissues or areas of tissue in the patient's brain, but not necessarily the whole brain, that the current being delivered to the patient's head is intended to treat. In one embodiment, the target tissue is defined or indicated by reference to a volume or an area where a specific function or pathology is localized. In one embodiment, the target tissue is located in the brain periphery, in a region near the cranium surface, or in a region proximal to the skull. In one embodiment, the target tissue may include motor regions or sensory regions or processing regions or cognitive regions. In one embodiment, the target tissue is specific by gyri or gyrus, including when a specific gyri or gyrus are linked with a specific function or pathology. In one embodiment, the target tissue may be defined based on the location of the ischemic tissue on perfusion hemodynamic imaging. In one embodiment, the target tissue is identified with assistance of perfusion hemodynamic scan.

In some embodiments, the cap 12 is fully configured with an electrode array 18 comprising electrodes positioned at every hole location 14. In such an embodiment, a subset of the electrodes may be selected based on which montage is suitable. In some embodiments, a user selects the montage, and hence electrode subset, via the user interface based on stroke imaging results. In some embodiments, a user inputs via the user interface details of the stroke imaging results and the system 10 will prompt a user with a suggested montage, and hence electrode subset, to select. In some embodiments, the system 10 is configured to receive as input stroke imaging results, analyze the input via algorithmic means, machine learning, artificial intelligence, or the like, and automatically select a montage, and hence electrode subset, to utilize. In some embodiments, the stroke imaging results may be from any suitable imaging type, including but not limited to, profusion scan, CT, MRI, ASPECTS score, and the like.

In some embodiments, the gel injection mechanism can be configured to automatically inject gel to a subset of or all of the electrodes in the electrode array. In some embodiments, the system 10 can perform an impedance check of the electrodes to determine if impedance and/or electrode to skin contact is poor, and can alert a user via an alarm system comprising an audio, visual, and/or haptic alarm via the user interface, controller computing device, and/or cap. In some embodiments, the system 10 can alert to a specific electrode that needs more gel based on an impedance measurement. In some embodiments, the system 10 can automatically inject more gel to a specific electrode based on an impedance measurement.

In some embodiments, the gel injection mechanism comprises one or more reservoirs to hold a gel supply, conduits from the one or more reservoirs to each electrode location, and any suitable manifolds and/or valves along the conduits for controlling injections of gel to the electrodes. In some embodiments, the gel injection mechanism comprises a reservoir per electrode with conduit from each reservoir to each gel injection site.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

FIG. 1C and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1C depicts an illustrative computer architecture for a computer 28 for practicing the various embodiments of the invention. The computer architecture shown in FIG. 1C illustrates a conventional personal computer, including a central processing unit 2850 (“CPU”), a system memory 2805, including a random-access memory 2810 (“RAM”) and a read-only memory (“ROM”) 2815, and a system bus 2835 that couples the system memory 2805 to the CPU 2850. A basic input/output system containing the basic routines that help to transfer information between elements within the computer, such as during startup, is stored in the ROM 2815. The computer 28 further includes a storage device 2820 for storing an operating system 2825, application/program 2830, and data.

The storage device 2820 is connected to the CPU 2850 through a storage controller (not shown) connected to the bus 2835. The storage device 2820 and its associated computer-readable media, provide non-volatile storage for the computer 28. Although the description of computer-readable media contained herein refers to a storage device, such as a hard disk or CD-ROM drive, it should be appreciated by those skilled in the art that computer-readable media can be any available media that can be accessed by the computer 28.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 28 may operate in a networked environment using logical connections to remote computers through a network 2840, such as TCP/IP network such as the Internet or an intranet. The computer 28 may connect to the network 2840 through a network interface unit 2845 connected to the bus 2835. It should be appreciated that the network interface unit 2845 may also be utilized to connect to other types of networks and remote computer systems.

The computer 28 may also include an input/output controller 2855 for receiving and processing input from a number of input/output devices 2860, including a keyboard, a mouse, a touchscreen, a camera, a microphone, a controller, a joystick, or other type of input device. Similarly, the input/output controller 2855 may provide output to a display screen, a printer, a speaker, or other type of output device. The computer 28 can connect to the input/output device 2860 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 2820 and RAM 2810 of the computer 28, including an operating system 2825 suitable for controlling the operation of a networked computer. The storage device 2820 and RAM 2810 may also store one or more applications/programs 2830. In particular, the storage device 2820 and RAM 2810 may store an application/program 2830 for providing a variety of functionalities to a user. For instance, the application/program 2830 may comprise many types of programs such as a word processing application, a spreadsheet application, a desktop publishing application, a database application, a gaming application, internet browsing application, electronic mail application, messaging application, and the like. According to an embodiment of the present invention, the application/program 2830 comprises a multiple functionality software application for providing word processing functionality, slide presentation functionality, spreadsheet functionality, database functionality and the like.

The computer 28 in some embodiments can include a variety of sensors 2865 for monitoring the environment surrounding and the environment internal to the computer 28. These sensors 2865 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

In one embodiment, the target tissue is identified with the assistance of structural and hemodynamic imaging including but not limited to CT or MRI brain, CT or MR angiography and CT or MR perfusion.

In one embodiment, in subjects receiving stroke imaging compromising of CT or MRI protocol includes but is not limited to non-contrast CT (NCCT), multiphase CT angiography and CT perfusion (CTP) or MRI, MR angiography and MR perfusion (MRP) processed through RAPID or other automated acute stroke software. In one embodiment, NCCT or MRI brain is used to rule out acute intracranial hemorrhage appearing as hyper density. In one embodiment, the tissue with rCBF <30% on CTP or ADC<620 is considered ischemic core and the hypoperfusion lesion volume is the entire ischemic region with Tmax≥6 sec. In one embodiment, CT angiography (CTA) or MR angiography (MRA) identifies large and medium vessel occlusion.

In one embodiment, the target tissue or regions typically indicates a portion of the brain defined as ischemic by perfusion scan or by presence of vessel occlusion on CTA/MRA or clinical presentation.

In one embodiment, predefined positioning or montages may be used to position plurality of electrodes on the target location. In one embodiment, montages can be selected from the list including but not limited to middle cerebral artery (MCA) M1 branch, MCA inferior branch (M2-I), MCA superior branch (M2-S), Anterior Cerebral artery (ACA), Posterior Cerebral Artery (PCA), Posterior Inferior Cerebellar Artery (PICA), Internal Carotid Artery (ICA). In one embodiment, these montages are based on computational modeling of 7 electrical fields concentrated over different parts of the vascular territories including but not limited to electrical field covering territory of MCA-M1 branch, electrical field covering territory of MCA-M2 superior branch, electrical field covering territory of MCA-M2 inferior branch, electrical field covering territory of ACA, electrical field covering territory of PCA, electrical field covering territory of PICA, and electrical field covering territory of ICA. For example, in a subject with the main branch of MCA ischemia, the electrical field is delivered only to the brain region supplied by the MCA. In one embodiment, a major portion of the electrical field concentrates in the cerebral spinal fluid surrounding the brain where the blood-brain barrier and leptomeningeal collateral blood vessels exist and therefore may have a vasodilatory effect on blood vessels by stimulating the release of vasodilators from perivascular neurons and the endothelial lining of cerebral vasculature, such as calcitonin-gene related peptide (CGRP), prostaglandin E2, vasoactive intestinal peptide (VIP), and nitric oxide (NO). It also results in the activation of nitric oxide synthase in endothelium and secretion of NO, resulting in vasodilation.

In one embodiment, enhancement of CBF and CBV and evidence of CSF hyperintensity marker are utilized as biomarkers of stimulation effects on collateral perfusion. The enhancement of collaterals could lead to better delivery of endogenous thrombolytics to the face of clot promoting recanalization.

In one embodiment, an integrated user-friendly interface is designed that utilizes the stroke imaging data to automatically select the appropriate electrode positioning or montage and performs an on-line rapid modeling of the electrical field via which the appropriateness of the selected montage with maximal coverage of the ischemic tissue by the electrical field is verified.

The controller may be any type of computer or controller suitable to comprise and run a control program. The controller has means for setting the parameters of the treatment such as duration and intensity of stimulation amplitude. A visual output, such as a screen of user interface may indicate such values as the set parameters for the treatment as well the current values of the treatment parameters, or other values that may be provide useful operating information to the user, such as the electrode impedance measured by an impedance monitor. The controller may also implement treatment programs, such as programs involving a ramp-up, ramp-down period which slowly increases or the stimulation amplitude over a set period of time up to the set stimulation amplitude or down to 0 mA, in order to limit any discomfort to the patient.

In some embodiments, the controller with user interface has a single lead extending to the plurality of electrodes 18. In an alternative embodiment, the system may be made smaller and more portable by eliminating cords and combing the 1×1 current generator and HD interface into a single current generator.

The electrical stimulation system 10 further comprises a current generator. In one embodiment, the controller is in communication with the current generator and may control and receive feedback from the current generator. The current generator provides a low-dose (low current) electrical stimulation impulse through at least 2 electrodes 18 to the subject. In one embodiment, the current generator is able to provide a sustained direct current with little to no variability in amperage. By sustained, it is meant that the current generator is capable of providing the current according to the varying requirements of the control program as described above. Additionally, in one embodiment, the current generator is capable of providing an alternating current at a known frequency and amplitude in order to measure electrical impedance of the electrodes as described below.

The user interface allows the user to select the treatment parameters including the desired montage, duty cycle, intensity and duration of the treatment. In one embodiment, the treatment parameters may be tailored and custom-programmed for each subject based on his or her particular disorder, symptoms, and other physiological or other concerns. In one embodiment, the user interface notifies the user of low battery, low-quality stimulation due to high impedance or poor electrode-skin contact as described below, etc.

In one embodiment, the duty cycle may be controlled on a time basis, that is, initiated on a given time and cycle at a predetermined interval thereafter until finished or stopped. In one embodiment, the duty cycle may be controlled in terms of the total time of each “on” cycle. In one embodiment, the current is provided for a range between 10 to 60 minutes continuously. In one embodiment, the current is provided for 20 minutes continuously. With regard to the “off” cycle, the current is off for a range between 10 to 60 minutes continuously in between the “on” cycles. In one embodiment, the current may be applied in at least one cycle. In one embodiment, the current may be applied in a single cycle of 20 minutes. In one embodiment, the current may be applied in at least two cycles. In one embodiment, the current may be applied in at least two cycles of 20 minutes on and 20 minutes off. In one embodiment, the current may be applied in at least three cycles. In one embodiment, the current may be applied in at least three cycles of 20 minutes on and 20 minutes off.

In one embodiment, the stimulation intensity is ranging from about 1 mA-4 mA. In one embodiment, the current may be applied in a single cycle of 20 minutes with a stimulation intensity of 1 mA. In one embodiment, the current may be applied in a single cycle of 20 minutes with a stimulation intensity of 2 mA. In one embodiment, the current may be applied in at least two cycles of 20 min on and 20 min off with a stimulation intensity of 1 mA. In one embodiment, the current may be applied in at least two cycles of 20 min on and 20 min off with a stimulation intensity of 2 mA. In one embodiment, the current may be applied in at least three cycles of 20 min on and 20 min off with a stimulation intensity of 1 mA. In one embodiment, the current may be applied in at least three cycles of 20 min on and 20 min off with a stimulation intensity of 2 mA.

The controller may be programmed to apply current according to a suitable ramp up from 0 mA when turning on and a similar ramp down to 0 mA when turning off. The ramp duration may be set at any time. In one embodiment, ramp duration may be ranging between 10 seconds to 10 minutes. In one embodiment, ramp duration may be 30 seconds. Ramp functionality may be employed to enhance patient comfort and safety which may be compromised by the sudden application of full current.

In some embodiments, the system includes a step of measuring electrical impedance of the electrodes 18. Impedance checking is used to ensure that the electrodes have good contact with the subject's skin. Good electrode-skin contact ensures accurate, efficient delivery of the electrical current, and thus maximizes the effectiveness of the therapy. Impedance checking can be done in several ways.

In one embodiment, the system may perform electrical impedance checking by any method currently known to those skilled in the art or later developed. In one embodiment, electrode impedance measurement involves calculating the electrical impedance value by measuring a voltage across two electrodes. The two electrodes may each be signal measurement or current delivery electrodes or may be a measurement or delivery electrode and a reference or return electrode. Impedance is the complex form of electrical resistance, that is, impedance is the electrical-resistance to sinusoidal, alternating current (AC). Impedance values take on a complex form containing both a magnitude as well as a phase, which indicates the lag between the current and voltage. Impedance can be calculated as a function of both the magnitudes and the phases of the voltage, current, and impedance. In various embodiments of the present invention, the calculation is very similar to traditional Ohm's law and calculates impedance by dividing the measured voltage by the known current. The phase component describes the fraction of the lagging wave that has been completed by the when it reaches the same reference point as the first signal, in the present case that reference point is the electrode. The calculation of an electrode's impedance involves supplying an electrical current to the electrode at a known frequency and amplitude, and measuring the voltage across that electrode and another electrode, in the first step, an electrical current is supplied to the first electrode. Once the current is being applied at the known frequency and amplitude, the system is able to take the required voltage measurement across the current-supplied electrode and another electrode, and calculate the impedance of that electrode to which the current is applied. Thereafter, the process is repeated for the other electrode to get impedance measurements for each of them. Some embodiments may involve simultaneously supplying a current at known amplitude and frequency to two electrodes, and measuring the voltage, thus providing a total impedance for the two electrodes combined. In such embodiments, the first electrode's calculated impedance is subtracted from the total impedance of the two electrodes to obtain the second electrode's impedance value. In many other embodiments however, the impedance values are measured individually for each electrode by supplying a current to each electrode in turn, as described above. In embodiments utilizing an electrode array, such as the previously described 4×1 array, the electrodes in each array are typically and preferably employed as a single electrode, or rather a single device, in such embodiments, the electrodes may be individually addressable, but are more often a single passive device wherein there is a single anodal electrode and the cathode is divided into separate parts, for example 4 parts in the 4×1 array. For purposes of the electrode impedance measurement described above with electrode array embodiments, when two electrodes are used, typically, such impedance measurements are performed between and/or among two separate arrays, and not between and/or amongst individual electrodes in a single array.

In one embodiment, the system compromises data storage for treatment data/reporting, including features that might allow a report exported and/or communicated to an electrical medical record.

The electrical stimulation system 10 is designed to be user friendly and employed rapidly by physicians, technologists, and any health care staff who have completed training and certification in safe use. In one embodiment, training includes instructions on different components of device 10, installing and assembling the components, charging device (or checking the battery charge) 10 prior to the first use, recharging device 10 after each procedure, verifying device 10 is charged prior to each procedure, recognizing and addressing the different Warning/Error indications and specific instructions for returning device 10 to the sponsor in the event of an error notification that cannot be addressed by the site personnel, or a failure of device 10 to charge after three hours. All device trainings are documented in a training log that are maintained in the site regulatory binder.

In one embodiment, warning/error indications include but not limited to battery light flashing blue, indicating that the battery is low but can still perform at least one procedure, battery light steady red, indicating battery low error—not enough battery power to run a complete procedure, beeps as additional ways to communicate with the user, and etc.

All embodiments of the present invention are designed to help improve the subject's quality of life. Early improvements may be measured by 1) normalized change in neurologic deficit from baseline to 24 h (normalized delta NIHSS—linear variable, analyzed with means and 95% CIs; and 2) degree of neurologic deficit at 24 h (NIHSS (National Institutes of Health Stroke Scale)-quasi-linear variable, analyzed with means and 95% CIs). Other improvements of greatest interest that is explored are: 1) degree of disability at 90 days, assessed across all 7 levels of the modified Rankin Scale (mRS)—ordinal variable, analyzed with medians (IQRs) and means (95% CIs); 2) functional independence (mRS 0-2) at 90 days—binary variable, analyzed with rates and 95% CIs; 3) granular degree of disability at 90 days (AMC Linear Disability Scale)—linear variable, analyzed with means and 95% CIs; and 4) health-related quality of life EuroQol (EQ-5D)—linear variable, analyzed with means and 95% CIs.

### Method of Use

The present invention provides a method to provide a low-dose electrical current, transcranially, to a specific area of the subject's brain for sustained relief of indications such as acute ischemic stroke via the system 10 described above. The method described herein focuses primarily on the application for treating acute ischemic stroke, but the systems and methods may also be used for the treatment of other indications, including but not limited to collateral enhancement for chronic ischemia as in intracranial steno-occlusive disease due to atherosclerosis or Moya Moya disease, etc. In one embodiment, the system and methods according to the present invention are non-invasive, portable, user friendly, capable of delivering the treatment rapidly and efficiently requiring minimal set-up.

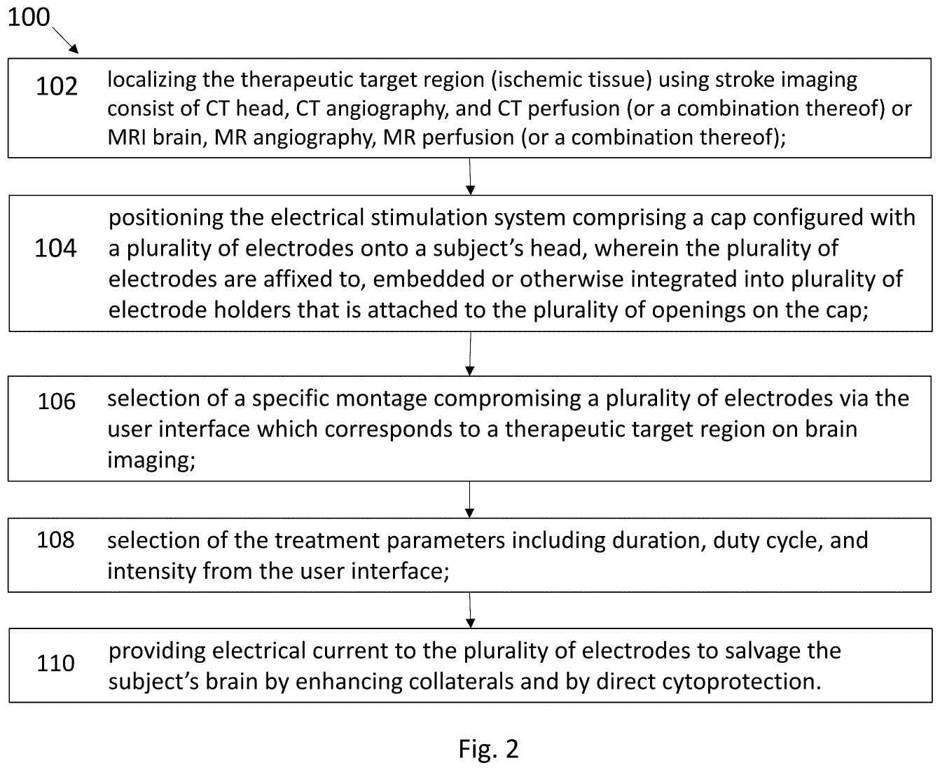

Referring now to FIG. 2, an exemplary method 100 of using the system is depicted. Method 100 begins with step 102, wherein stroke imaging is obtained to rule out brain hemorrhage and locate the therapeutic target region. In step 104, the system comprising a cap with a plurality of electrodes onto a subject's head, wherein the plurality of electrodes are affixed to, embedded or otherwise integrated into plurality of electrode holders that is attached to the plurality of openings on the cap. The electrodes and their holders may be incorporated into the wearable cap vs added and reconfigures via the holes in the cap. In step 106, a specific montage compromising a plurality of electrodes is selected via the user interface which corresponds to a therapeutic target region on stroke imaging. In step 108, the treatment parameters including duration, duty cycle, and intensity is selected from the user interface. In step 110, electrical current is delivered via the plurality of electrodes to salvage the subject's ischemic brain. In this step, the system begins to supply an electrical current at a known, steady amperage, through at least two electrodes, across the subject's cranium. In one embodiment, this current is targeted at a particular area of the subject's brain which corresponds to the location of ischemia and occluded vessel on stroke imaging.

Subject, or patient, demographic data and medical histories may be obtained or utilized to determine eligibility for treatment and for analysis of factors for data categorization, etc. Eligible subjects or their legally authorized representatives (LAR) are provided with explicit written informed consent.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out exemplary embodiments of the present invention, and are not to be construed as limiting in any way the remainder of the disclosure.

### Example 1

Stroke is a devastating condition and continues to be a leading cause of adult death and disability (Benjamin E. J. et al., 2017, Circulation 135(10): e146-e603). Current therapeutic strategies for AIS focus on timely restoration of blood flow by recanalization of the occluded artery to salvage penumbral tissue, using pharmacologic fibrinolysis and/or endovascular thrombectomy (ET) (Campbell, B. C. et al., 2015, Lancet Neurol. 14 (8): 846-854; Adeoye, O. et al., 2011, Stroke, 42 (7): 1952-1955). However, the reperfusion strategy has limitations that would be aided by a neuroprotective intervention. An important minority of early-arriving patients are ineligible for both IV tPA (due to lytic contraindications) and ET (due to difficult vascular access, large core, peripheral target occlusion beyond catheter reach in patients with medium vessel occlusion (MVO)). Other patients are non-responders: IV tPA only achieves reperfusion in only 15-40 percent of large vessel occlusions (LVOs); ET fails to achieve substantial reperfusion in 20-30%. Furthermore, even among reperfusing patients, rates of excellent outcome are low, only 20-25% mRS 0-1, due to infarct growth prior to, and following reperfusion (Saver, J. L. et al., 2016, JAMA 316 (12): 1279-1288; Haussen, D. C. et al., 2016, J. Neurointerv. Surg. 8 (2): 117-121; Goyal, M. et al., 2016, Lancet, 387 (10029): 1723-1731). Therefore, even in the modern stent retriever era, the need to develop additional therapies exists. Neuroprotective therapies interrupt the cellular, biochemical and metabolic process that mediate hypoxic and reperfusion cellular injury. Neuroprotective therapies could serve as alternative treatments for patients who harbor salvageable penumbra in whom reperfusion therapies are contra-indicated or have failed, and as early bridging therapy in pre-reperfusion patients, preserving more viable penumbra to rescue intervention by reperfusion. While multiple candidate neuroprotective agents have failed in translation from animal to human studies, important lessons have been learned (Tymianski, M., 2013, Stroke, 44 (10): 2942-2950; Saver, J. L. et al., 2015, N. Engl. J. Med. 372 (6): 528-536). Two intervention properties that offer greater prospect of success are: 1) pleiotropic effects, interdicting multiple pathways in the ischemic cascade, and 2) delivery by a direct transcranial, rather than intravascular, route, with fast and direct delivery to neural tissues regardless of cerebral perfusion. Transcranial direct current stimulation (tDCS) offers promise as just such an approach. Furthermore, few trials of neuroprotection in human have used penumbral imaging for patient selection (Warach, S. et al., 2000, Ann. Neurol. 48 (5): 713-722) and importantly, the recent trials of thrombectomy in late-arriving patients have shown a significant benefit from recanalization therapy in patients with a salvageable penumbra despite their late presentation from last known well time (Albers, W. G. et al., 2017, ClinicalTrials.gov; Nogueira, R. G. et al., 2017, N. Engl. J. Med.). Therefore, the use of penumbral imaging to limit the patient selection to those for whom the intervention is most likely to be of benefit is crucial in studies of neuroprotection and this study utilizes such an approach.

tDCS is a non-invasive neuromodulation method that delivers a weak electrical current to the brain via scalp electrodes (Woods, A. J. et al., 2016, Clin. Neurophysiol., 127(2):1031-1048). Rather than directly eliciting a neuronal response, tDCS modulates neuronal excitability in regions of the brain depending on the polarity of stimulation. By altering the resting membrane potential cathodal tDCS (C-tDCS) reduces neuronal excitability [in contract to anodal tDCS (A-tDCS) which increases excitability] (Nitsche, M. A. et al., 2005, J. Physiol. 568(Pt 1):291-303; Nitsche, M. A. et al., 2003, Clin. Neurophysiol. 114(4):600-604). tDCS has been extensively investigated in humans for decades, as a neuromodulatory intervention to treat depression and diverse other neuropsychiatric disorders, as a neuroplasticity-enhancing intervention for chronic stroke patients, and as a tool to change cognition and behavior in healthy individuals (Brunoni, A. R. et al., 2012, Brain Stimul. 5(3):175-195; Dmochowski, J. P. et al., 2013, Neuroimage 75:12-19; Teichmann, M. et al., 2016, Ann. Nurol. 80(5):693-707; Wessel, M. J. et al., 2015, Front Hum. Neurosci. 9:265). Thus far, tDCS has been found safe and tolerable with no reported serious adverse events across multiple clinical and preclinical studies (Bikson, M. et al., 2016, Brain Stimul., 9(5):641-661; Chhatbar, P. Y. et al., 2017, Brain Stimul. 10(3):553-559). In addition to these established applications of tDCS, tDCS is of substantial promise for acute cerebral ischemia, based on preclinical studies. In pre-clinical studies, multiple investigators in several independent labs worldwide have found evidence of a neuroprotective effect of tDCS in different animal models of acute cerebral ischemia (Table 1):

Table 1: Shows the detailed description of the studies using tDCS as a neuroprotective method

| | | | | Polarity | | | |
|---|---|---|---|---|---|---|---|
| | | Onset of | | and | | | |
| | | stim | | location of | | Total | |
| Study | Type of | following | | the center | Intensity | Duration | |
| authors | subjects | MCAO | | electrode | (mA) | of stim | Total N |
| Notturno | Rat | 45 | min | Cathode | 0.2 | 120 and | 48 |
| et al et. | MCAO | | | over | (density | 180 min | |
| al 2013 | model | | | cortical | of 2.86 | (alternating | |
| | | | | surface | mA/cm2) | 15′on- | |
| | | | | overlying | | 15′off) | |
| | | | | ischemia | | | |
| Peruzzotti- | Mice | 30 | min | Cathode or | 0.25 | 40 min | 137 |
| Jametti | MCAO | | | anode over | (density | (alternating | |
| et. al | model | | | cortical | of 5.5 | 20′on- | |
| 2010 | | | | surface | mA/cm2) | 20′off) | |
| | | | | overlying | | | |
| | | | | ischemia | | | |
| Baba et | Rat | 60 | min | Electrode | 0.1 and | 3 Days or | 107 |
| al. 2009 | MCAO | | | over | 0.2 with | 1 week | |
| | Model | | | cortical | frequencies | | |
| | | | | surface | of 0, | | |
| | | | | overlying | 2, 10, 50 | | |
| | | | | ischemia | | | |
| Kim et al. | Rat | Day | 2 | Cathode or | 0.1 | 30 min | 61 |
| 2010 | MCAO | | | anode over | | daily for 2 | |
| | model | | | cortical | | weeks | |
| | | | | surface | | | |
| | | | | overlying | | | |
| | | | | ischemia | | | |
| Glickstein | Rat | 5 days | | Stimulation | 0.01- | 60 | 98 |
| et. al | MCAO | prior to | | of the | 0.02 | min(alternating | |
| 1999 | model | MCAO | | fastigial | | 1 sec on-1 | |
| | | | | nucleus of | | sec off) | |
| | | | | cerebellum | | | |
| Reis et.al | Rat | Immediately | | Cathode | 0.01- | 60 min | 99 |
| 1997, | MCAO | after, up | | over the | 0.02 | | |
| 1991 | model | to 10 days | | fastigial | | | |
| | | prior to | | nucleus of | | | |
| | | MCAO | | cerebellum | | | |
| Zhang | Rat | 3-5 | min | Cathode | 0.07- | 60 min | 44 |

| | | | | | | |
|---|---|---|---|---|---|---|
| et. al | MCAO | | over the | 0.1 | (alternating | |
| 1993 | model | | fastigial | | 1 sec on- | |
| | | | nucleus of | | 1 sec off) | |
| | | | cerebellum | | | |
| Yamamoto | Rat | Immediately | Cathode | 0.07- | 60 min | 41 |
| et. al | MCAO | | over the | 0.1 | (alternating | |
| 1993 | model | | fastigial | | 1 sec on- | |
| | | | nucleus of | | 1 sec off) | |
| | | | cerebellum | | | |
| Burger | Rat | Immediately | Stimulation | Unknown | 60 min | |
| et. al | MCAO | | of the | | | |
| 1990 | model | | fastigial | | | |
| | | | nucleus of | | | |
| | | | cerebellum | | | |

Neuroprotective Mechanism of tDCS Based on Experimental Pre-Clinical Models of Acute Cerebral Ischemia

### Inhibition of Peri-Infarct Excitatory Depolarizations

During the acute stages of cerebral ischemia, an excitotoxic cascade is triggered by the excess glutamate and other excitotoxic amino acids that are released as the result of cellular necrosis (Harukuni, I. et al., 2006, Neurol. Clin. 24(1):1-21). Experimental models of acute middle cerebral artery occlusion (MCAO) have shown that the excitotoxicity generates recurrent spontaneous waves of depolarization also known as peri-infarct depolarizations (PIDs) (Nakamura, H. et al., 2010, Brain 133(Pt 7):1994-2006; Hossmann, K. A. et al., 1996, Cerebrovasc. Brain Metab. Rev. 8(3):195-208). The PIDs occur soon after the MCA occlusion and spread across the penumbra to the normally perfused tissue. The infarct growth correlates with the number and duration of PIDs and the basis for this relationship has been related to: 1) an abnormal vasoconstriction in response to depolarization 2) an imbalance between increased metabolic overload, induced by the depolarization, and blood supply in acute ischemic stroke (Hartings, J. A. et al., 2003, J. Neurosci. 23(37):11602-11610). Notturno et al. studied the effect of C-tDCS on PIDs in 3-vessel occlusion rat stroke model, with cumulative stimulation durations of 120 and 180 mins (15′ on-15′ off cycles) (Natturno, F. et al., 2014, J. Neurol. Sci., 342(1-2):146-151). They found that C-tDCS was applied to the ischemic MCA territory significantly reduced PIDs, and reduced infarct volume by 20-30%. They found no effect on brain edema between the stimulated and sham groups and no tDCS induced macroscopic or microscopic lesion or hemorrhagic transformation.

### Anti-Inflammatory, Anti-Apoptotic and Angiogenic Effect

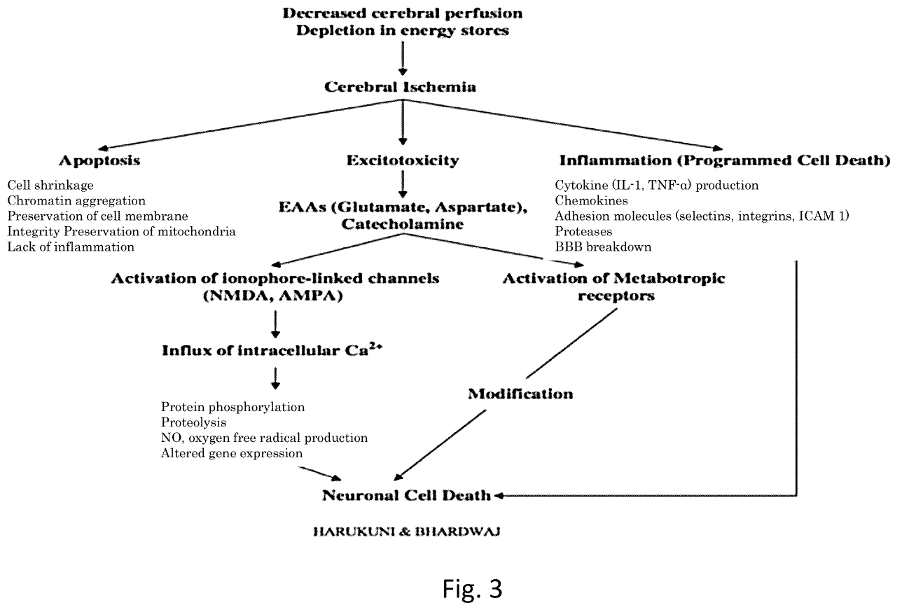

Beyond the activation of the excitotoxic cascade, an inflammatory response and programmed cellular apoptosis results in a secondary damage and expansion of the irreversibly damaged core (FIG. 3) (Harukuni, I. et al., 2006, Neurol. Clin. 24(1):1-21). Therefore, suppression of the innate pro-inflammatory cells and the cellular apoptotic cascade results in the reduction of the infarct size and cerebral edema. Peruzzotti et al. studied tDCS in acute stroke mice models with MCAO with cathodal hemispheric tDCS applied for 40 minutes (20′on-20off-20′on) (Peruzzotti-Jametti, L. et al., 2013, Stroke 44(11):3166-3174). Cathodal stimulation of the ischemic hemisphere reduced final infarct size, with lowering of cortical glutamate synthesis, downregulation of N-methyl-D-aspartate (NMDA) receptor (NR2B) expression, and reduction in peri-ischemic inflammatory response and apoptotic markers. Furthermore, a significant functional amelioration and improvement of cerebral edema were observed even when stimulation was applied hours after the MCAO. No macroscopic or microscopic lesion or hemorrhagic transformation induced by tDCS was found.

Baba et al. showed in their study that a low-frequency (2 and 10 Hz) electrical cortical stimulation exerts neuroprotective effects reflecting by attenuation in both necrotic and apoptotic cell deaths, blockade of microglial/astrocytic activation and expression of trophic factors. They also demonstrated an increase in cerebral blood flow of the electrically stimulated animals (Baba, T. et al., 2009, Stroke 40(11):e598-605). No neuroprotective effect was observed at higher frequency (50 Hz).

### Preservation of Neuronal Axons

Kim et al. studied the neuroprotective effect of cathodal and anodal tDCS on axons and myelin integrity. They delivered tDCS for 30 minutes two days after MCAO. They found significant amelioration of axonal damage and preservation of white matter axonal integrity in rat models of cerebral ischemia. They demonstrated such effect only after anodal stimulation (Kim, S. J. et al., 2010, J. Korean Med. Sci. 25 (10): 1499).

### Direct Vasodilatory Effect

In addition to the direct neuroprotective effects, tDCS may confer benefit in AIS via direct vasodilation and collateral blood flow enhancement. In fact, Fox et al. found a direct vasodilatory effect of electrical stimulation when applied over the basilar artery (Fox, J. L. et al., 1974, J. Neurosurg. 41(5):582-589). This effect was more pronounced with cathodal compared to anodal stimulation.

### Preliminary Studies

Experience with Transcranial Neuromodulation in Human Subjects

Inventors of the present invention at UCLA have extensive experience with transcranial neuromodulation using direct electrical and magnetically induced currents in human subjects. Over the past 10 years, transcranial neuromodulation were performed in over 500 subjects (Lacoboni, M., 2008, J. Physiol. Paris 102(1-3):31-34; Aziz-Zadeh, L. et al., 2004, Eur. J. Neurosci. 19(9):2609-2612; Lacoboni, M., 2006, Neuropsychologia 44(13):2691-2699; Dunn, W. et al., 2016, Schizophr. Res. 174(1-3):189-191; Rassovsky, Y. et al., 2015, Schizophr. Res. 165(2-3):171-174; Dunn, W. et al., 2017, J. Neural Transm. (Vienna) 124 (9): 1145-1149). This substantial experience provides a firm foundation for undertaking tDCS studies in patients with acute ischemic stroke.

a Meta-Analysis of Preclinical Studies Using tDCS as Neuroprotection in Acute Cerebral Ischemia

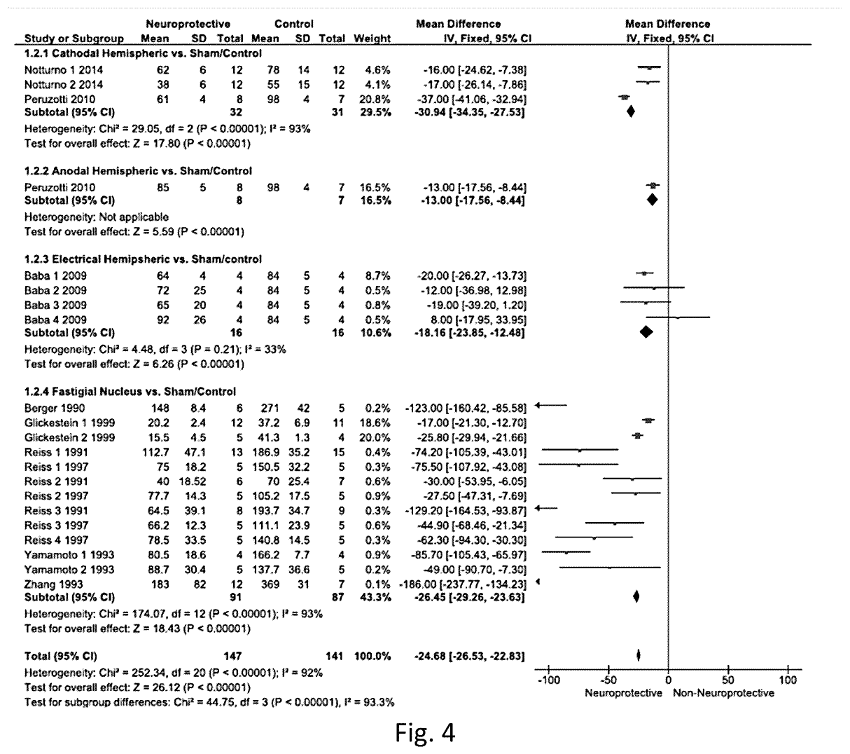

To assess the neuroprotective effect of tDCS in AIS, a systematic review of all preclinical acute cerebral ischemia studies was performed using tDCS as a neuroprotective method. The systematic search identified 21 controlled comparisons of tDCS in preclinical acute cerebral ischemia models, including a total of 256 animals, all with middle cerebral artery occlusion (Notturno, F. et al., 2014, J, Neurol Sci., 342(1-2):146-151; Peruzzotti-Jametti, L. et al., 2013, Stroke 44(11):3166-3174; Reis, D. J. et al., 1991, J. Cereb. Blood Flow Metab., 11 (5): 810-818; Reis, D. J. et al., 1998, Brain Res., 780(1):161-165; Yamamoto, S. et al., 1993, J. Cereb. Blood Flow Metab. 13(6):1020-1024; Zhou, P. et al., 2001, J. Neurochem. 79(2):328-338; Glickstein, S. B. et al., 2001, Brain Res. 912(1):47-59). Hemispheric cathodal stimulation was used in 3 experiments (32 animals), hemispheric anodal stimulation in 1 experiment (8 animals), Electrical stimulation in 4 experiments (———animals) and fastigial nucleus stimulation in 13 experiments (91 animals). Overall, tDCS reduced final infarct volume by 24.68 mm³ (95% CI 26.53-22.83, P<0.00001). Only mild heterogeneity of effect by stimulation type was noted (12=90.5%), with the infarct reduction magnitudes relatively larger with cathodal hemispheric stimulation—30.94 mm³ (95% CI 34.35-27.53, P<0.00001), followed by targeted fastigial nucleus stimulation-26.4 mm³ (95% CI 29.26-23.63, P<0.00001), then hemispheric electrical stimulation-18.16 (95% CI 23.85-12.48, P<0.00001), and hemispheric anodal stimulation-13 mm³ (95% CI 17.56-8.44, P<0.00001) (FIG. 4).

These findings demonstrate that tDCS significantly reduces final infarct volume across animal preclinical studies. The greatest neuroprotective effect is observed with the C-tDCS directly applied to the ischemic hemisphere (hemispheric cathodal stimulation). Therefore, the study of hemispheric C-tDCS in patients with acute ischemic stroke is warranted.

Vascular Response Associated with tDCS

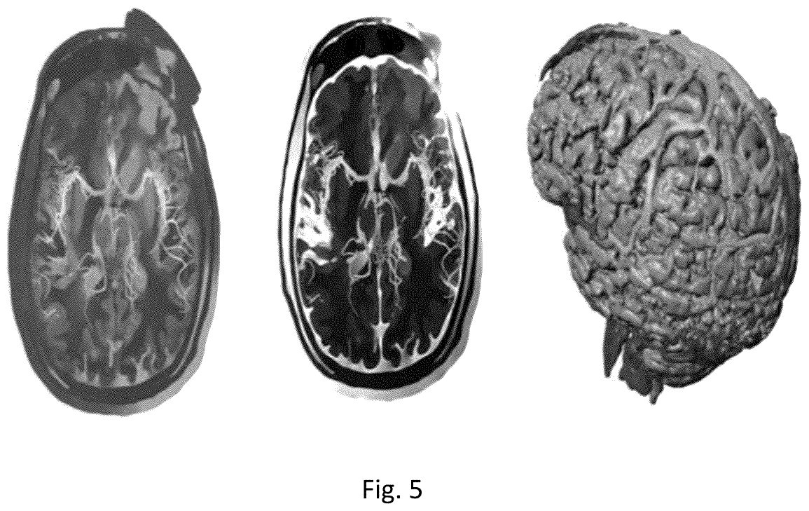

A preliminary study in animal models by Marom Bikson demonstrated that tDCS produces vasodilation of cerebral vessels (FIG. 5). Furthermore, the vasodilatory response to tDCS is evident by its known mild dose-dependent effect in causing skin erythema. The cerebral vasodilatation is likely partially due to non-specific polarization of vascular system. Bikson and colleagues also demonstrated that 10 minutes of tDCS resulted in up-regulation of endothelial nitric oxide synthase (eNOS) gene expression and increase production of nitric oxide (NO), a known vasodilator (Bikson, M., 2017). Samdani et al. have demonstrated in their study that the endothelial NO upregulation favorably affects outcomes by the accentuation of the cerebral blood flow and attenuation of platelet aggregation, platelet adhesion, and NMDA current (Samdani, A. F. et al., 1997, Stroke, 28(6):1283-1288). These findings support that tDCS could augment blood flow through the stimulated vasculature. Leptomeningeal collateral networks, peripherally located, are particularly accessible to the electric field. The potential vasoactive effects of tDCS also raise the possibility that stimulation affects the blood-brain barrier. While no hemorrhagic transformation with C-tDCS was noted in preclinical models, this potential effect supports the approach of undertaking dose escalation safety trial as the first study in AIS patients, even though all studied dose tiers are within ranges safe in chronic stroke and other brain disease patients (Woods, A. J. et al., 2016, Clin. Neurophysiol. 127(2):1031-1048; Chhatbar, P. Y. et al., 2017, Brain Stimul. 10(3):553-559).

### Enhancement of Cerebral Blood Flow and Collateral Perfusion

C-tDCS results in a primary vasodilatory response by stimulating the release of vasodilators from perivascular neurons and the endothelial lining of cerebral vasculature. Transcranial electrical stimulation has been shown to stimulate the peri-vascular neurons of leptomeningeal/pial arteries secreting vasoactive substances, such as calcitonin-gene related peptide (CGRP), prostaglandin E2, vasoactive intestinal peptide (VIP), and nitric oxide (NO). It also results in the activation of nitric oxide synthase in endothelium and secretion of NO, resulting in vasodilation. This mechanism was deemed responsible for the ameliorating effect of C-tDCS on the frequency and severity of vasospasm in preclinical models of subarachnoid hemorrhage. Given that leptomeningeal/pial vessels are more superficially located closer to the meninges and skull, they receive a much higher electrical current density than the deeper vasculature and neuronal tissue. Furthermore, due to their larger innate diameter, higher transmural pressure, and stronger endogenous electrical field, electrical current is likely to have a larger enhancing effect on leptomeningeal arteries compared to smaller parenchymal arterioles and capillaries. The electrical field concentration has been modeled across the subarachnoid space and shown that it is consistently over two orders of magnitude greater than the electrical field within the brain parenchyma. This higher concentration of electrical field allows the electrical current to maximize its influence on leptomeningeal arteries, thus making it an attractive collateral and cerebral blood flow enhancing technique.

### Feasibility of Neuroprotection Therapies in Acute Ischemic Stroke

Researchers at UCLA have extensive experience with the conduct of clinical trials of drug and device neuroprotective therapies for acute ischemic stroke, both as standalone interventions and as a complement to reperfusion treatment. Recently the NIH Field Administration of Stroke Therapy-Magnesium phase 3 trial, enrolling 1700 patients was completed (Saver, J. L. et al., 2015, N. Engl. J. Med. 372(6):528-536). Of particular relevance to the current study, trials of transcranial delivery of acute neuroprotective intervention using laser (Huisa, B. N. et al., 2013, Int. J. Stroke 8(5):315-320), and trials of collateral enhancement interventions (Shuaib, A. et al., 2011, Stroke 42(6):1680-1690) and trials and studies using MRI penumbral imaging and permeability imaging as technical efficacy and safety end points was conducted.

Frequency and Features of AIS LVO and MVO Patients with Substantial Salvageable Penumbra Who do not Undergo ET

Among the eventual target populations for tDCS as a neuroprotective intervention in acute LVO and MVO AIS, several are suboptimal for inclusion in an initial, dose escalation safety trial. Ambulance patients (prehospital neuroprotection) are in a chaotic environment unsuited for close safety observations; ED patients pre-ET (bridging neuroprotection) have intense time-pressure for usual care making safety-emphasis studies difficult; ET failure patients (rescue neuroprotection) have potential complications from their failed procedure confounding study interpretation. In contrast, ET-ineligible patients (pure neuroprotection) are an attractive cohort for a safety study, as they are under close observation, free of intense time-pressure for ET, and free of course outcome being strongly determined by ET outcome. For the current study, therefore, a study to delineate the frequency, clinical characteristics, and outcomes of patients with substantial salvageable penumbra who are ineligible for ET in the modern stent retriever era was performed (Bahr Hosseini, M. W. G. et al., 2017, European Stroke Organization Conference). Patients were recognized as having substantial salvageable penumbra when their perfusion lesion volume (tissue with a delay in contrast arrival to peak concentration (Tmax) of ≥6 sec on perfusion-weighted image (PWI)) was ≥1.2 times larger than the ischemic core volume (tissue with a low mean water diffusivity or apparent-diffusion coefficient (ADC)≤620 μm²/s on diffusion-weighted image (DWI)) (PWI-DWI Mismatch) (Butcher, K. S. et al., 2005, Stroke 36(6):1153-1159; Furlan, A. J. et al., 2006, Stroke, 37(5):1227-1231; Hacke, W. et al., 2005, Stroke, 36(1):66-73). The patients were then categorized with substantial salvageable penumbra as having an LVO if MRA or CTA showed occlusion of the intracranial internal carotid (ICA), the M1 segment of the middle cerebral artery (MCA), the vertebral artery (VA), or the basilar artery (BA). Patients were categorized as having an MVO if MRA or CTA showed occlusion of the M2 segment of the MCA, the A1 segment of the anterior cerebral artery (ACA), or the P1 segment of the posterior cerebral artery (PCA); or if perfusion imaging indicated occlusion an M3 segment of the middle cerebral artery by showing a perfusion lesion volume of at least 10 ml in an appropriate territorial distribution. Among 174 consecutive AIS patients, 29 (17%) were LVO and MVO patients with substantial salvageable penumbra who did not undergo ET. Mean age was 81 (±13), 45% were female, and median NIHSS was 11 (IQR 5-19). The prevalence of LVO was 59% (19/29) and MVO 41% (12/29). Patients with ICA, M1 and M2 occlusions constituted most of the cases (78%). The four most common reasons for not pursuing ET intervention were: distal occlusion (28%), large infarct core (16%), low NIHSS (16%), temporally advanced core injury evident from fluid attenuated recovery (FLAIR) changes on MRI or frank hypodensity on CT (13%). Other reasons included: chronic occlusion of the cervical internal carotid artery precluding intracranial access (9%); poor pre-stroke baseline function (9%); intracranial occlusion judged to be a chronic atherosclerotic occlusion (6%); extracranial vessel tortuosity precluding intracranial access (3%). Median time from LKN to imaging was 410 min (IQR 198-615). Mismatch ratio was median 5.6 (IQR 2-infinite), salvageable penumbra volume mean was 54 ml (±63), and ischemic core volume was mean 20 ml (±31). Severe disability or death at discharge (mRS 4-6) occurred in 72%. These findings demonstrate that, even in the modern stent retriever era, one in six acute cerebral ischemia patients presents with substantial salvageable penumbra judged not appropriate for ET. This population is more than sufficiently common for this study.

The materials and methods employed in these experiments are now described.

### Trial Design and Methods

This invention is a prospective, single-center, dose-escalation safety, tolerability and feasibility study of tDCS in acute stroke patients with substantial salvageable penumbra due to a large or medium vessel occlusion who are ineligible for endovascular therapy. The primary safety endpoints are symptomatic intracranial hemorrhage during the 24-hour period after stimulation. Secondary measures of safety include asymptomatic intracranial hemorrhage, early neurological deterioration, 3-month mortality and all 12 serious adverse events. Tolerability is judged based on the percentage of the patients completing the protocol-assigned stimulation treatment and secondarily, the rate and severity of cutaneous, neurologic, nociceptive or other adverse effects are assessed. Feasibility endpoints analyze the speed with which tDCS is implemented. Finally, signals of potential efficacy are explored by examining the imaging biomarkers, including penumbral salvage, collateral enhancement, and infarct growth, and clinical outcomes of early neurologic deficit evolution, and 3-month global disability and health-related quality of life.

### Technology

HD tDCS Device and Stimulation Parameters

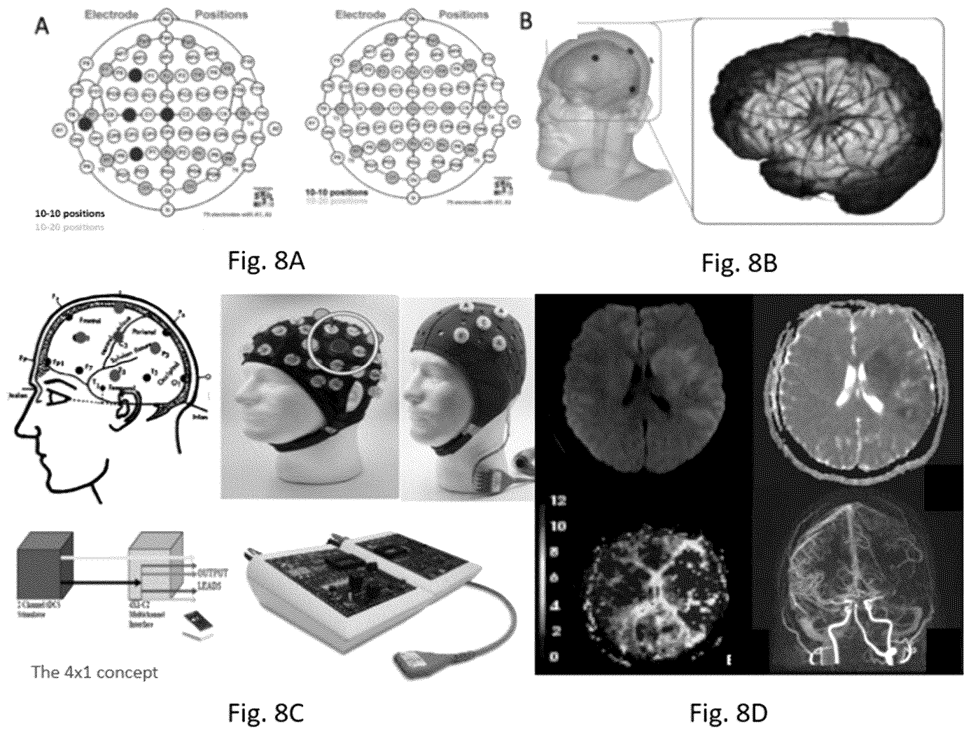

The study employs a Soterix™ high-definition DC-Stimulator. This tDCS unit consists of a stimulator, 4×1 HD interface and an adjustable cap with pre-made openings that quickly and easily fits different head sizes. (FIGS. 8A-D). The cap is loaded with plastic electrode holders. These are filled with conductance gel (Signa® gel), 1 cm² electrodes are placed in the holders, and the holder then locks.

### Montage

The electrode positioning montage are a 4 to 1-ring configuration, with the center or active electrode connected to cathode and the 4 reference or return electrodes connected to anode.

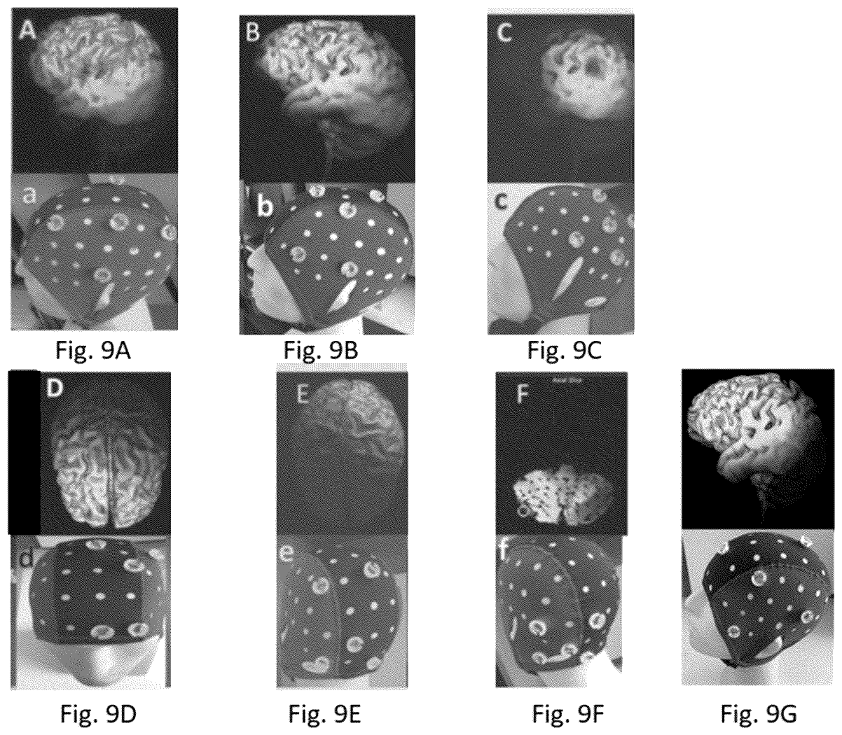

For electrodes positioning location, one of the 7 predefined positionings is used according to the location of the vascular occlusion: middle cerebral artery (MCA) M1 branch, MCA inferior branch (M2-I), MCA superior branch (M2-S), Anterior Cerebral artery (ACA), Posterior Cerebral Artery (PCA), Posterior Inferior Cerebellar Artery (PICA). These electrode positionings are based on computational modeling of 6 electrical fields concentrated over different parts of the aforementioned vascular territories: Electrical field covering territory of MCA-M1 branch: anodes over F3, Cz, P3, T7; Cathode over C3. (FIG. 9A).
Electrical field covering territory of MCA-M2 superior branch: Anodes over C1, F1, F7, T7; Cathode over FC3 (FIG. 9B).
Electrical field covering territory of MCA-M2 inferior branch: Anodes over C3, P3, T7, P7; Cathode over CP5 (FIG. 9C).
Electrical field covering territory of ACA: Anodes over FPz, FCz, FC3, FP1; Cathode over F1 (FIG. 9D).
Electrical field covering territory of PCA: Anodes over Pz, Iz, PO9, P3; Cathode over O1 (FIG. 9E).
Electrical field covering territory of PICA: Anodes over O1, O9, Ex1, Ex5; Cathode over PO9 (FIG. 9F).
Electrical field covering territory of ICA: Anodes over the Fpz, Cz,CP5,F9; Cathode: F3 (FIG. 9G).

### Sterility and Quality Assurance

Prior to stimulation, all the electrodes are monitored for any sign of damage such as chipping. The electrodes are discarded after being exposed to 5 cycles of stimulation. Any metal contact is avoided to contact the electrodes. The stimulation cap and the electrodes are sanitized prior to each subject use.

### Device Training

Device use is performed by physician investigators, technologists, and research staff who have completed training and certification in safe tDCS use. Training includes instructions on different components of the device, installing and assembling the components, charging the device prior to the first use, recharging the device after each procedure, verifying the device is charged prior to each procedure, recognizing and addressing the different Warning/Error indications (e.g., battery light flashing blue, indicating that the battery is low but can still perform at least one procedure, battery light steady red, indicating battery low error—not enough battery power to run a complete procedure, etc.) and specific instructions for returning the device to the sponsor in the event of an error notification that cannot be addressed by the site personnel, or a failure of the device to charge after three hours. All device trainings are documented in a training log that are maintained in the site regulatory binder.

### Subjects

Twenty-four to 48 acute ischemic stroke patients with substantial salvageable penumbra due to a large or medium vessel occlusion who are ineligible for endovascular therapy and meet study inclusion/exclusion criteria are enrolled from Ronald Reagan Medical Center (RRMC) Emergency Department or RRMC inpatients.

Based upon acute stroke patient referral rates to UCLA over the past 3 years and the retrospective study looking at the thrombectomy-ineligible patients with substantial penumbra in an 8-month period (1 in 6 acute ischemic stroke patients), it is anticipated that 8-16 enrollments can be done per year. Therefore, it is estimated that the study enrollment takes 3 years to complete. Enrolled subjects are randomized to active versus sham stimulation in 3:1 ratio.

### Entry Criteria

Inclusion criteria are 1) new focal neurologic deficit consistent with AIS 2) NIHSS≥4 or NIHSS<4 in the presence of disabling deficit (a deficit that, if unchanged, would prevent the patient from performing basic activities of daily living such as bathing, ambulating, toileting, hygiene, and eating or returning to work); 3) age>18; 4) presence of any cortical vessel occlusion including ICA, branches of MCA, Anterior Cerebral artery (ACA), Posterior Cerebral artery (PCA), Posterior-Inferior cerebellar artery (PICA); 5) presence of salvageable penumbra with Tmax>6 sec/ischemic core volume (ADC<620 μm2/s or rCBF <30%)≥1.2 6) patient ineligible for IV tPA, per national AHA/ASA Guidelines 7) patient ineligible for ET per AHA/ASA national Guidelines-one or more of: poor pre-stroke functional status (mRS score>1), mild neurological symptoms (NIHSS<6), large ischemic core (ASPECTS<6), thrombectomy not technically performable due to severe vessel tortuosity, cervical artery chronic occlusion, or other unfavorable angioarchitectural features that preclude endovascular access to the target intracranial vessel. 8) subject is able to be treated with tDCS within 24 hours of last known well time; 9) a signed informed consent is obtained from the patient or patient's legally authorized representative.

Exclusion criteria are 1) acute intracranial hemorrhage 2) evidence of a large Ischemic core volume (ADC<620 μm2/s or rCBF<30%)≥100 3) presence of tDCS contraindications-electrically or magnetically activated intracranial metal and non-metal implants. 4) severe MR contrast allergy or renal dysfunction with eGFR<30 ml/min, precluding MRI gadolinium or CT iodine contrast 5) pregnancy 6) signs or symptoms of acute myocardial infarction, including EKG findings, on admission 7) suspicion of aortic dissection on admission 8) history of seizure disorder or new seizures with presentation of current stroke 9) evidence of any other major life-threatening or serious medical condition that would prevent completion of the study protocol including attendance at the 3-month follow-up visit 10) concomitant experimental therapy 11) preexisting scalp lesion at the site of the stimulation or presence of skull defects (may alter current flow pattern) 12) preexisting coagulopathy, consist of platelet count of ≤100, INR≥3, PTT≥90.

### Biological Variables

The entry criteria have been designed to be broadly inclusive of biological variables that may modify disease course and treatment response, including enrollment of all adults of any age, both males and females (except pregnant females), and all weights compatible with MR-scanning. Pregnant women are excluded as the safety of tDCS has not been established in pregnancy. A pregnancy test is performed prior to enrollment in women of childbearing age. Children are excluded because of the rarity of diagnosis in children in the acute time window, and greater brain plasticity and recovery in younger individuals. Given the uncommon availability for enrollment and very different course, including children in trial could differentially favor or unfavor one study arm, and make interpretation of findings challenging.

With regards to the time window, patients treatable within 24 hours of last known well are included, as the DAWN and DEFUSE 3 trials have shown that patients in the 6-24 hr window with imaging evidence of substantial salvageable penumbra are responsive to acute stroke therapies (Albers, W. G., 2017, Clinical trials.gov; Nogueira, R. G. et al., 2017, N. Engl. J. Med. 378(1):11-21). Sites of vessel occlusion include any cortical vessel occlusion including distal branches of MCA (M3, M4), ACA, PCA, and PICA, in addition to LVO's (ICA, M1, and M2 branches of MCA), and regarding the severity of deficits, NIHSS<4 is included in the presence of a disabling deficit, in addition to NIHSS≥4. This patient population with more distal vessel occlusion and less severe deficits are the likely patients not proceeding to thrombectomy even in the current expanded treatment era, and who are also as informative regarding the main safety, tolerability, and feasibility endpoints of the study.

### Enrollment and Consent

All acute ischemic stroke patients within 24 hours of their symptoms onset who present to Roland Reagan Medical Center (Emergency Department or inpatient hospital if they are already admitted for a different indication) and meet the study inclusion/exclusion criteria are identified by a study physician-investigator and subsequently offered enrollment in the trial. Prospective subjects are provided with written and verbal information regarding the nature of the study, the procedures and evaluations involved, and the potential risks and benefits. All participating patients or their legally authorized representatives (LAR) provide explicit written informed consent. Not all adult subjects have the capacity to give informed consent. The likely range of impairment includes stupor and aphasia. By interviewing the patient, the investigators assess whether the affected individual understands the central elements of the study procedures and has the capacity for informed consent, using the recommended approach of the institution's Institutional Review Board, such as the UCLA Office of Human Research Protection Decision-Making Capacity Assessment Tool.

Patients with capacity to consent are invited to participate by the PI. In patients without the capacity to consent, the patient's LAR are asked to provide consent for participation. If the LAR is not physically available but reachable through the phone at the time of enrollment, the informed consents are sent to the LAR via fax or email after discussing the details of the study via phone. Then the LAR returns the signed/e-signed form to the PI, again either via tax or email.

The investigator informs the patient or legally authorized representative of the availability of the study as follows: “You (your relative) is having a stroke. We are doing a research study of a new treatment for stroke. Here is an informed consent form that describes the study. Please read it. After you are finished, I will answer any questions you may have.” Once subjects or their legally authorized representatives have read and understood the IRB-approved consent form, and had all their questions answered, written informed consent are elicited.

### Dose Tiers and Randomization

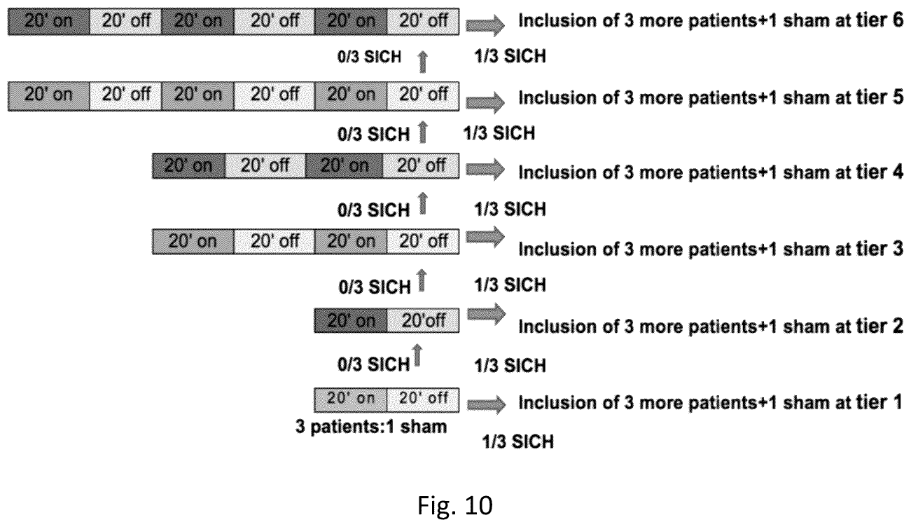

A traditional 3+3 (rule-based, modified Fibonacci) dose escalation design is implemented, with 3:1 randomization to active treatment vs. sham control. There are 6 dose tiers, reflecting increasing intensity and duration of stimulation: Tier 1-1 mA, single 20-min cycle; Tier 2-2 mA, single 20 min cycle; Tier 3-1 mA, 2 cycles of 20 min/20 min off; Tier 4-2 mA, 2 cycles of 20 min/20 min off; Tier 5-1 mA, 3 cycles of 20 min/20 min off; Tier 6-2 mA, 3 cycles of 20 min/20 min off (FIG. 10). Patients in the sham stimulation arm at all the tiers have the cap and electrodes in place, and switches moved but without any delivered electrical stimulation. While the highest dose tier in this study is expected to be fully safe, based on preclinical and clinical studies (Woods, A. J. et al., 2016, Clin. Neurophysiol. 127 (2): 1031-1048; Bikson, M. et al., 2016, Brain Stimul. 9 (5): 641-661), since this is a first-in-human study in acute stroke, a formal escalation to higher dose tiers is prudent.

### Management During and Post Stimulation Period

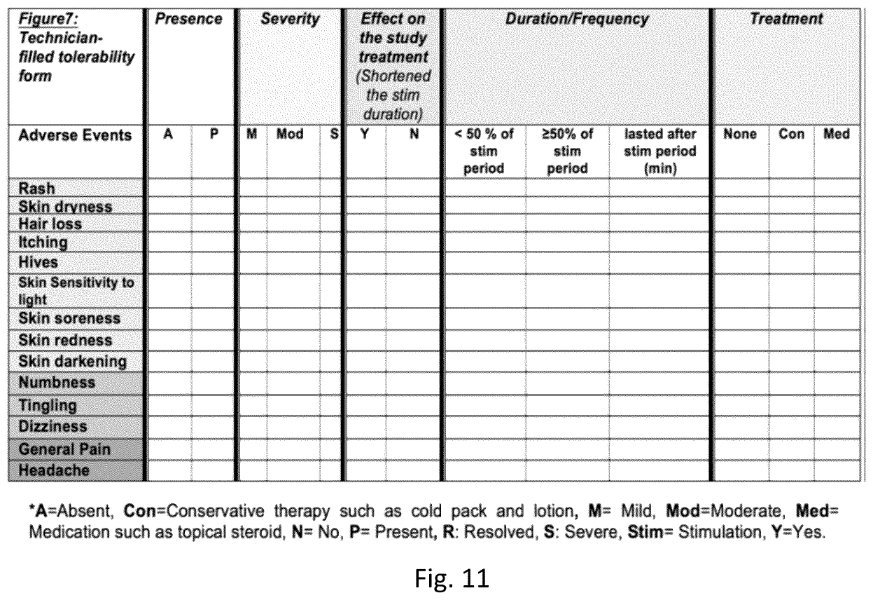

After randomization and initiation of the stimulation, the patient is transported to the neurological intensive care unit or UCLA Stroke unit for close observation. Subsequent care is continued in these settings, including medical management per national guidelines for acute ischemic stroke management issued by the American Stroke Association (Powers, W. J. et al., 2015, Stroke 46 (10): 3020-3035). Frequent neuro-checks are performed by neuroscience nurses with extensive experience in monitoring acute stroke patients. NIHSS is obtained after each 20-minute stimulation cycle, and at 2-hour and 24-hour post-stimulation. Before and after each 20-minute treatment cycle, the technologist performs a visual inspection of the skin and rate degree of any potential erythema under the electrode. Then, at the end of each 20-minute stimulation session, a tolerability form is completed based on validated cutaneous, neurological, and pain items of the PRO-CTAE (Patient-Reported Outcomes version of the Common Terminology Criteria for Adverse Events) (FIG. 11) (Basch, E. et al., 2014, J. Natl. Cancer Inst. 106(9); Dueck, A. C. et al., 2015, JAMA oncol. 1(8):1051-1059). A separate tolerability form is completed by the patient at the end of each stimulation cycle (20 minutes). All patients undergo a multimodal MRI or CT including standard parenchymal images, non-invasive angiography, and perfusion studies at 2-hour and 24-hour following the end of the stimulation tier.

Study visits and data acquisition

| | | End of each 20- | | | | | | | |
|---|---|---|---|---|---|---|---|---|---|
| | | min stimulation | | | | | | | |
| | | cycle | | | 2- | 24- | Day | Day | Day |
| | Baseline | 1 | 2 | 3 | hr | hr | 4 | 30 | 90 |
| Consent | x | | | | | | | | |
| History/Exam | x | | | | | | | | |
| Routine Labs | x | | | | | | | | |
| Vital Signs | x | x | x | x | | | | | |
| Patient tolerability | | x | x | x | | | | | |
| form | | | | | | | | | |
| Technician tolerability | x | x | x | x | | | | | |
| form/Skin Inspection | | | | | | | | | |
| Multimodal MRI or CT | x | | | | x | x | | | |
| NIHSS | x | x | x | x | x | x | | | x |
| Modified Rankin Scale | x | | | | | | x | x | x |
| (mRS) | | | | | | | | | |
| Barthel Index (BI) | | | | | | | | | x |
| EuroQol (EQ-5D) | | | | | | | | | x |
| AMC Linear Disability | | | | | | | | | x |
| Scale | | | | | | | | | |
| Interval serious adverse | | x | x | x | x | x | x | x | x |
| evens | | | | | | | | | |
| Concomitant/Interval | x | x | x | x | x | x | x | x | x |
| medications/procedures | | | | | | | | | |

### Baseline Evaluation

Demographics (age, sex, race)
Last known well time
Past medical/surgical history including vascular diagnoses and risk factors (stroke, TIA, carotid stenosis, myocardial infarction, atrial fibrillation, peripheral arterial disease, hypertension, diabetes, dyslipidemia)
Medications, including antithrombotics, antihypertensives, statins, anti-arrhythmics Family history of vascular disease;
Tobacco (including timing, duration, amount), alcohol, and illicit drug use;
Vital signs (systolic blood pressure, diastolic blood pressure, pulse, respiratory rate, oxygen saturation);
Premorbid global disability (modified Rankin Scale);
Neurological deficits severity (NIHSS);
Laboratory results (CBC, platelet count, glucose, lytes, INR, PTT, LFTs.
   During tDCS Treatment
NIH Stroke Scale (NIHSS)
Tolerability/AE Form-Technologist, Tolerability Questionnaire-Patient
   Early (2 Hours) after tDCS
NIHSS
Multimodal MRI/CT
Interval serious adverse events
Interval medications/procedures
   Late (24 Hours) after tDCS
NIHSS
Multimodal MRI/CT
Interval serious adverse events
Interval medications/procedures

### Day 4

Modified Rankin Scale (mRS)
Interval serious adverse events
Interval medications/procedures

### Day 30

mRS (phone)
Interval serious adverse events
Interval medications/procedures

### Day 90

NIHSS
Modified Rankin Scale (mRS)
AMC Linear Disability Scale (granular disability)
Barthel Index (BI) (activities of daily living)
EuroQol (EQ-5D) (health-related quality of life)
Interval serious adverse events
Interval medications/procedures

### Imaging Assessments

### Baseline

Emergent multimodal MRI or CT is currently acquired as the routine initial imaging study in all acute stroke patients at UCLA without contraindications such as the presence of a pacemaker or metal implant. A 1.5 T or 3 T scanner equipped with echo-planar imaging capability is used for rapid acquisition of diffusion and perfusion scans. The standard clinical MRI protocol includes Gradient Recall-Echo (GRE), DWI, FLAIR (Fluid-Attenuated Recovery Image), PWI and MR angiography. The ADC (apparent-diffusion coefficient) values derived from DWI acquisition (b=0, 1000 s/mm2 applied in each of three principal gradient directions) is used to delineate the volume of ischemic core. The tissue with ADC values of ≤620 μm²/s is considered ischemic core, indicative of tissue with advanced, irreversible bioenergetic compromise. FLAIR delineates early parenchymal signal abnormality associated with ischemia and slow retrograde flow in leptomeningeal collaterals appearing as FLAIR vascular hyperintensity (FVH). The GRE sequence is used to evaluate the presence of intracranial hemorrhage and deoxygenated leptomeningeal collaterals appearing as GRE vascular hypointensity (GVH). PWI is acquired with sequential T2*-weighted (gradient echo) EPI time sequence scanning. Early in the time series, a bolus (0.1 mmol/kg) of MRI contrast material is rapidly infused (5 ml/see through an 18 or larger gauge angiocatheter) using a power injector. The perfusion lesion volume (tissue at risk volume) is the region with Tmax≥6 sec (Tmax=the time delay from the arrival of contrast to its peak concentration at the tissue vasculature) (Albers, G. W. et al., 2017, Int. J. Stroke 12(8):896-905; Bahr Hosseini, M. et al., 2017, Neuropharmcology). Intracranial CEMRA (Contrast-Enhanced MRA) identifies large and medium vessel occlusion.

In patients receiving CT, the standard clinical CT protocol includes non-contrast CT (NCCT), multiphase CT angiography, CT perfusion (CTP) processed through RAPID software. NCCT is used to rule out acute intracranial hemorrhage appearing as hyper density. The tissue with rCBF <30% on CTP is considered ischemic core and the perfusion lesion volume (tissue at risk volume) is the region with Tmax>6 sec. CT angiography (CTA) identifies large and medium vessel occlusion.

### 2 h and 24 h Post-Stimulation

Follow-up MR or CT imaging is obtained 2 h and 24 h post-stimulation (supported by Radiology Dept. research funds). MRI Imaging sequences include DWI/FLAIR/GRE/PWI/CEMRA and CT imaging sequences consist of NCCT/CTA/CTP. RAPID image processing software is applied to the above images to quantify, at the baseline, 2 h, and 24 h time-points: 1) Ischemic Core volume, 2) Perfusion lesion volume, and 3) Penumbra volume (perfusion volume-core volume). From these values, the following measures are constructed: 1) Early penumbra preservation: Volume of baseline penumbra tissue not progressing to ischemic core at 2 h; 2) Penumbral salvage: Volume of baseline penumbra tissue not progressing to ischemic core at 24 h; 3) Early collateral flow enhancement: Perfusion lesion volume at baseline—Perfusion lesion volume at 2 h; 4) Sustained collateral flow enhancement: Perfusion lesion volume at baseline—Perfusion lesion volume at 24 h; 5) Early infarct growth: Ischemic core lesion volume at 2 h—Ischemic core lesion volume at baseline; and 6) Final infarct growth: Ischemic core lesion volume at 24 h—Ischemic core lesion volume at baseline. Additional location and extent of ischemic injury at baseline, 2 h, and 24 h is rated using the MRI ASPECTS scoring system, with regions considered involved if diffusion restriction is present in more than 20% of the region or CT ASPCETS (Demchuk, A. M. et al., 2005, Neuroimaging Clin. N. Am. 15(2):409-419; Saver, J. L. et al., 2015, N. Engl. J. Med. 372(24):2285-2295). In patients receiving MRI, location and extent of FVH and GVH is rated using the FVH-modified ASPECTS and GVH modified ASPECTS scales (Mahdjoub, E. et al., 2017, AJNR Am. J. Nuroradiol.).

### Adverse Events

### Serious Adverse Events (SAEs)

All serious adverse events occurring during the 90 days of study participation is recorded. A serious adverse event is any adverse event that is fatal, is life-threatening, is permanently or substantially disabling, requires or prolongs hospitalization, or requires medical or surgical intervention to prevent one of the above outcomes (Bikson, M. et al., 2016, Brain Stimul. 9(5):641-661).

### Symptomatic Intracranial Hemorrhage

The lead safety endpoint adverse event is symptomatic intracranial hemorrhage (SICH), defined using the SWIFT PRIME trial criteria (Saver, J. L. et al., 2015, N. Engl. J. Med. 372(24):2285-2295): an increase of 4 or more points on the NIHSS within 24 hours of stimulation associated with parenchymal hematoma type 1 (PH1), parenchymal hematoma type 2 (PH2), remote intraparenchymal hemorrhage (RIH), subarachnoid hemorrhage (SAH), or intraventricular hemorrhage (IVH). In addition, all hemorrhages, both symptomatic and asymptomatic, are separately classified and analyzed by radiologic subtype, as hemorrhagic infarction type 1 (HI1), hemorrhagic infarction type 2 (HI2), PH1, PH2, RIH, SAH, or IVH (Saver, J. L. et al., 2015, N. Engl. J. Med. 372(24):2285-2295). A central neuroimaging core lab, blinded to treatment assignment, reviews all brain MRI scans obtained at 24 h and rate presence and type of radiologic hemorrhagic transformation. In addition, in patients who experienced worsening by 4 or more NIHSS points in the first 24 hours, any and all additional brain MRI or CT scans obtained during the 24 h time period is reviewed.

SICH is the primary safety endpoint of the current study, but the results are assessed on a variety of additional safety, feasibility, and tolerability results as well. After completion of the current study, the judgement of whether to proceed directly to a pivotal trial, to proceed to a larger safety and preliminary efficacy trial, or to not proceed with further development, rests on a considered and informed assessment of all outcome measures. It is important therefore to collect data on a wide range of safety endpoints and a wide range of measures of functional outcome, as is planned in this study. In making the selection of a primary safety endpoint, an emphasis was placed on ensuring patient safety throughout the course of the trial by choosing an endpoint with uncontestable clinical relevance (Symptomatic Intracranial Hemorrhage).

Additional Adverse Events with Specific Interrogation

In addition to general screening for all serious adverse events and focused elicitation of symptomatic intracranial hemorrhage events, the following events are specifically interrogated for and recorded in the case report forms: skin redness, scalp rash, hair loss, seizure, headache, sensitivity to light, new ischemic stroke, deep venous thrombosis, pulmonary embolism, pneumonia, acute M1.

### Safety Monitoring

### Data and Safety Monitoring Plan

The trial is monitored by an Independent Data and Safety Monitoring Board (DSMB). DSMB is assessed for the causal relationship of the serious adverse event to the study treatment as definite, probable, possible, unlikely, or unrelated. DSMB meets at the completion of each dose tier, review all safety data, and determine whether the study proceeds to the next dose tier. DSMB deliberations is guided by: 1) a formal stopping/escalation rule, based on the occurrence of the lead safety endpoint, symptomatic intracranial hemorrhage (SICH), and 2) The DSMB members' clinical judgement upon review of all other safety outcomes. In addition, DSMB monitors separately the patients who enroll in the study with a stroke scale 0-3 during the study as they are treated to determine if they have an increase in NIHSS of 2 or more or which is disabling.

Furthermore, Food and Drug Administration (FDA) is notified if there is one occurrence of SICH prior to repeating the tier or escalating to a higher tier.

### Statistical Design and Analyses Plan

### Sample Size

The study sample size derives from the use of the 3+3, rule-based, modified Fibonacci, dose escalation design, with 3:1 randomization to active treatment vs sham control. There are 6 dose tiers, reflecting increasing intensity and duration of stimulation (FIG. 10). The 3+3 study design (3 patients and 1 sham) with 6 dose tiers yields a sample size of at least 24 and potentially up to 48. The 3+3 design is the classical approach to dose escalation in first-in-human studies (Hansen, A. R. et al., 2014, Cancer Control 21(3):200-208; Panel, N. et al., 2009, Invest. New Drugs 27(6):552-556). While newer, adaptive designs for dose escalation trials have been developed, they are more complex and have limited advantages when major toxicities are not expected (Le Tourneau, C. et al., 2009, J. Natl. Cancer Inst. 101(10):708-720). Therefore, the rule-based, 3+3 design remains the dominantly employed approach in current dose-escalation studies. Every step is taken to avoid missing data by scheduling of the follow-up visits early in visit time windows and readiness to travel to the patient's location to perform needed assessments. Should any missing data occur, primary analyses is performed using multiple imputation, with sensitivity analyses including complete case and worst possible outcome analyses (Little, R. J. et al., 2012, N. Engl. J. Med. 367(14):1355-1360).

### Baseline Characteristics

The demographic and baseline clinical characteristics of the study population is delineated with standard descriptive statistics. Categorical variables describing the clinical history, examination findings, and initial treatment is summarized by frequencies. Continuous variables such as vital signs, laboratory results, and time variables is characterized by means, standard deviations, and 95% confidence intervals (CI). Ordinal and non-normally distributed variables (such as the NIHSS) is characterized by medians and interquartile ranges. Baseline characteristics is compared between the tDCS stimulation group with sham group to assess covariate balance. Wilcoxon Rank-Sum tests is used for continuous or ordinal variables; Fisher's exact tests and chi-square tests is used for grouped or nominal categorical variables.

### Safety and Tolerability

### Dose Escalation

The study Data and Safety Monitoring Board (DSMB) meet at the completion of each dose tier, review all safety data, and determine whether the study proceeds to the next dose tier. DSMB deliberations is be guided by: 1) a formal stopping/escalation rule, based on the occurrence of the lead safety endpoint, symptomatic intracranial hemorrhage (SICH), and 2) The DSMB members' clinical judgement upon review of all other safety outcomes. The formal dose escalation rule uses SICH frequency to gate the occurrence and pace of escalation through the 6 dose tiers. If no SICH occurs in the 3 active patients at a dose tier, enrollment may escalate to the next dose tier. If one SICH occurs, 3 more active (and 1 more control) patient is enrolled at that dose tier before escalation. If 2 SICHs occur at a dose tier, further study enrollment is held until detailed review by the DSMB (FIG. 8). In addition, whenever the formal SICH criteria for dose escalation has been met, the DSMB formally meet, review the SICH data and all other safety data, and advice regarding proceeding to the next tier, continuing at the current tier, or placing the study on hold.

### Primary Safety Endpoint Analysis

For the final statistical analysis of the primary SICH safety endpoint, a chi-square test is used to detect differences in the rate of SICH between the active treatment and sham patients and higher and lower dose tiers. The treatment is considered to have exhibited adequate safety in the current trial to proceed to future, larger, pivotal efficacy trials if tDCS results in lower or equivalent rates of SICH compared to sham.

### Secondary Safety Endpoint Analysis

In secondary safety endpoint analyses, the following is compared between the active treatment and sham patients, and between higher and lower dose tiers, using chi-squared tests: 1) Asymptomatic ICH by 24 h (intracranial hemorrhage not associated with NIHSS worsening≥4); 2) Early neurologic deterioration (worsening≥4 on NIHSS during the 24-hour period after stimulation, with or without intracranial hemorrhage); 3) All-cause mortality at day 90 (mRS); and 4) All serious adverse events.

### Tolerability Endpoint Analysis

The lead tolerability endpoint is completion of the protocol-assigned stimulation treatment without early cessation due to cutaneous, neurologic, nociceptive or other adverse effects. Experience with tDCS in post-stroke patients indicates only infrequent cutaneous (itching, tingling) adverse effects are likely to occur. Accordingly, for the current study, a patient is considered to have tolerated the procedure if at least 75% of the stimulus period was completed. The treatment is considered generally tolerable if, among all enrolled patients, tolerated procedures are achieved in ≥90% of patients, assessed with a one-sided p value of 0.025. Secondary tolerability endpoints is the rate and severity of cutaneous, neurological, and pain items of the technician-filled (FIG. 10) and patient-filled tolerability forms (Basch, E. et al., 2014, J. Natl. Cancer Inst. 106(9)), descriptively compared between active treatment and sham patients, and between higher and lower dose tiers.

### Feasibility Endpoint Analysis

A time-motion analysis and mock run-throughs are conducted prior to first enrollment. Nonetheless, processes to optimize rapid placement of the cap and electrodes continues to improve with experience gained from initially enrolled patients. The predefined success threshold for feasibility is median time from randomization to tDCS initiation≤10 minutes in the last 10 enrolled patients.

### Exploratory Efficacy Endpoint Analysis

### Exploratory Imaging Biomarker Efficacy Endpoint

This study is underpowered to definitively determine efficacy, so all imaging efficacy analyses is purely exploratory and descriptive. Imaging biomarker efficacy endpoints is characterized in the active and sham patients, and in higher and lower dose tiers, using means and 95% confidence intervals. The six imaging efficacy endpoints of greatest interest that is explored are: early and late penumbral salvage, early and late collateral flow enhancement, and early and late infarct growth.

### Exploratory Clinical Efficacy Endpoint

This study is underpowered to definitively determine efficacy, so all clinical efficacy analyses are purely exploratory and descriptive. Four clinical outcome measures were selected based on their reliability, familiarity to the neurologic community, and adaptability for use in patients who have had a stroke. These endpoints are: the modified Rankin Scale (mRS), a rating of global disability; the Barthel Index (BI), a measure of instrumental activities of daily living; the National Institutes of Health Stroke Scale (NIHSS), a measure of neurologic deficit severity; and the EuroQol (EQ-5D), an assessment of health-related quality of life; and AMC Linear Disability Scale, a granular degree of disability.

Clinical efficacy endpoints are characterized in the active and sham patients, and in higher and lower dose tiers. Early course clinical efficacy endpoints of greatest interest that is explored are: 1) normalized change in neurologic deficit from baseline to 24 h (normalized delta NIHSS—linear variable, analyzed with means and 95% CIs; and 2) degree of neurologic deficit at 24 h (NIHSS—quasi-linear variable, analyzed with means and 95% CIs). Final outcome clinical efficacy endpoints of greatest interest that is explored are: 1) Degree of disability at 90 days, assessed across all 7 levels of the modified Rankin Scale—ordinal variable, analyzed with medians (IQRs) and means (95% CIs); 2) Functional independence (mRS 0-2) at 90 days—binary variable, analyzed with rates and 95% CIs; 3) Granular degree of disability at 90 days (AMC Linear Disability Scale)—linear variable, analyzed with means and 95% CIs; and 4) Health-related quality of life (EQ-5D)—linear variable, analyzed with means and 95% CIs.

Additional studies were also performed and are detailed below. Further details can be found in Bahr-Hosseini et al. (High-definition Cathodal Direct Current Stimulation for Treatment of Acute Ischemic Stroke: A Randomized Clinical Trial. JAMA Netw Open. 2023; 6(6):e2319231. doi: 10.1001/jamanetworkopen.2023.19231), and Bahr-Hosseini et al. (Neurovascular-modulation: A review of primary vascular responses to transcranial electrical stimulation as a mechanism of action, Brain Stimulation: Basic, Translational, and Clinical Research in Neuromodulation, Volume 14, Issue 4, 837-847), each incorporated herein by reference in their entirety.

The study design was a traditional 3+3 (rule-based, modified Fibonacci) dose-escalation, with 3:1 randomization to active treatment vs sham control. The study was triple blinded with patients, treatment team, and outcome assessors blinded to the study treatment. A random allocation sequence table was generated by Biostatistics Core at UCLA using the random number generator in the R software version 4.0.5 (R foundation for statistical computing, Vienna Austria). The interactive web response system (REDCap) was used for allocation concealment and final randomization at the time of patients' enrollment. Per the 3+3 study design, a total enrollment of 24-48 patients and a total of 6 dose tiers were initially planned, increasing in intensity or duration of stimulation: Tier 1-1 mA, single 20-min cycle; Tier 2-2 mA, single 20 min cycle; Tier 3-1 mA, 2 cycles of 20 min/20 min off; Tier 4-2 mA, 2 cycles of 20 min/20 min off; Tier 5-1 mA, 3 cycles of 20 min/20 min off; Tier 6-2 mA, 3 cycles of 20 min/20 min off. Occurrence of symptomatic intracranial hemorrhage (SICH) determined the escalation pace. Dose escalation decisions were made in tandem with Data and Safety Monitoring Committee. The escalation guiding rules were: 1) absence of SICH in any of the three active patients at a given tier-escalate; 2) SICH in 1 of 3 active patients-enroll 4 additional patients (3 active, 1 sham) at that tier, escalate if no further SICH; 3) SICH in a 2^{nd} patient at a dose tier-trial stops. Patients in the sham stimulation arm had the cap and electrodes in place, but without any delivered electrical stimulation. A trained HD-tDCS technician who was not part of the care team performed the randomization and the study stimulation. Key entry criteria were patients with acute ischemic stroke within 24 hr from onset; imaging evidence of cortical ischemia; presence of salvageable penumbra; and ineligibility for reperfusion therapies (intravenous lytics and endovascular thrombectomy) (full entry criteria are available in Supplement Table 1). The rates of SICH at 24 hours and all serious adverse events were recorded for safety outcomes. The time from randomization to study stimulation initiation was recorded for the feasibility outcome. After randomization and during the stimulation, patients were monitored closely by the physician-investigator. The National Institute of Health Stroke Scale (NIHSS), a validated quantitative assessment tool to measure stroke-related neurological deficit, was obtained at the end of 20-minute stimulation cycle. Immediately following the stimulation cycle, a visual inspection of the skin and completion of a tolerability form based on validated cutaneous, neurological, and pain items of the PRO-CTAE (Patient-Reported Outcomes version of the Common Terminology Criteria for Adverse Events) were performed. The rate of patients completing the study stimulation period was recorded for tolerability outcome.

After the stimulation, patients were monitored in the Neurointensive Care Unit or Stroke Unit, and frequent neurologic assessment was performed by nurses with extensive experience in monitoring acute stroke patients. Specifically, patients were monitored for any signs of neurological worsening and development of new or worsening neurological symptoms. Subsequent care was continued in these settings, including medical management per national guidelines for acute ischemic stroke management issued by the American Stroke Association. Patients underwent multimodal MRI or CT at 2-4 hour (Early time point) and 24-30 hour (Late time point) following the end of the stimulation tier. Long-term clinical outcome measure was assessed using the modified Rankin Scale (mRS) of global disability at 90 days post-stimulation. The mRS assesses disability in stroke patients with score of 0 indicating no symptoms, 1 no significant disability despite mild residual symptom, 2 slight disability, 3 moderate disability but able to walk without assistance, 4 moderately severe disability needing assistance for walking, 5 disability requiring constant care for all needs; 6 death. As recruitment was slower than expected, related to the COVID pandemic, the study was stopped after enrollment of the 10th patients.

A total of 6 dose tiers were initially planned, increasing in intensity or duration of stimulation. The occurrence of symptomatic intracranial hemorrhage (SICH) determined the escalation pace through the tiers.

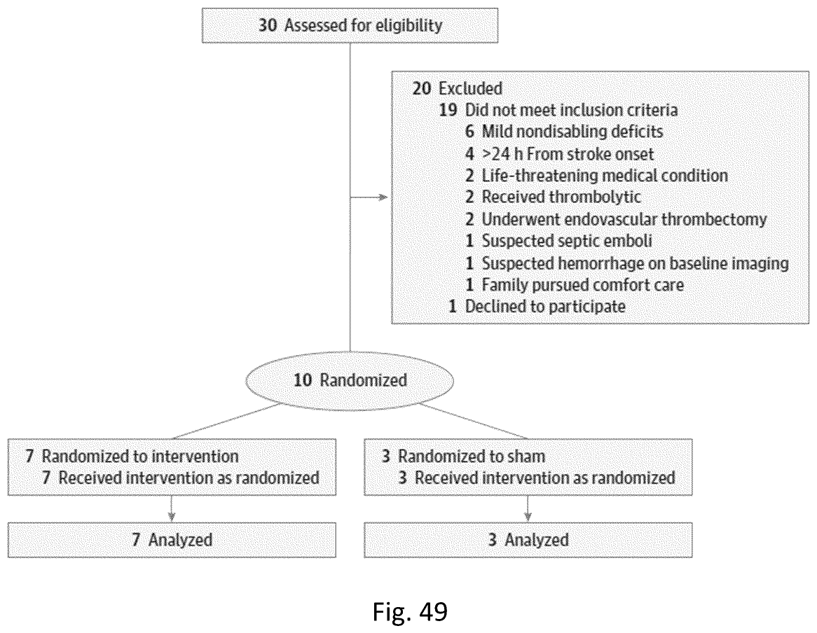

HD C-tDCS was delivered to the ischemic tissue using individualized montages. Six HD montages were predesigned to cover 6 vascular distribution-specific ischemic fields caused by occlusions of the middle cerebral artery (MCA) trunk, MCA superior division, MCA inferior division, posterior cerebral artery, anterior cerebral artery, and posterior inferior cerebellar artery. The coverage of ischemic regions by electric fields was estimated via computational modeling of the electrical current flow (FIG. 49).

The time from randomization to study stimulation initiation was recorded for the feasibility outcome. Tolerability was assessed with 2 end points: (1) the rate of patients completing the full neurological symptoms including dizziness or headache. Each item was scored as absent or present and subsequently graded for severity (mild, moderate, or severe), its effect on the stimulation protocol, the symptom's duration, and whether any intervention was done to remedy the symptoms.

The imaging biomarkers of neuroprotection and collateral enhancement were characterized at 2 to 4 hours (early time point) and 24 to 30 hours (late time point) and included improved perfusion (reduction in hypoperfusion region volume); collateral enhancement (increase in quantified relative cerebral blood volume [qrCBV]); and penumbral tissue salvage (tissue at risk not progressing to infarction). The modified Rankin Scale (mRS) was measured at 90 days for exploratory clinical efficacy analysis.

The demographic and baseline clinical characteristics of the study population were delineated with standard descriptive statistics. Categorical variables were summarized by frequencies. Continuous variables were characterized by means and standard deviations or median values with interquartile ranges (IQRs). Due to the descriptive nature of the primary feasibility and tolerability end points, tests of statistical significance were not performed.

The direct current flow models of various 4×1 high-definition electrode montages were generated on a health “standard” head using HD-Explore software (version 3.2, Soterix Medical Inc, New York). Six current flow patterns that matched the 6 commonly compromised vascular territories: 1) middle cerebral artery (MCA) trunk; 2) MCA superior division; 3) MCA inferior division; 4) posterior cerebral artery; 5) anterior cerebral artery; and 6) posterior inferior cerebellar artery. Following patient randomization, the appropriate montage with current flow pattern visually matching the location of hypoperfused ischemic region on MR or CT perfusion parametric map was selected. The following isotropic direct current electrical conductivities in (S m-1) were assigned: scalp (0.465), skull (0.01), CSF (1.65), gray matter (0.276), white matter (0.126), air (1e-7) electrodes (5.8e7), gel (0.3).

Imaging analyses were performed by a board-certified neuroradiologist with 10 years of experience who was blinded to the study treatment. MRI Imaging sequences included diffusion-weighted image (DWI)/fluid-attenuated inversion recovery (FLAIR)/gradient recall echo (GRE)/Dynamic susceptibility contrast (DSC) perfusion/contrast-enhanced MR angiography. CT imaging sequences consisted of non-contrast CT/CT angiography/CT perfusion. Clinical RAPID image processing software were applied to the above images to quantify, at the baseline, 2 h (Early time point), and 24 h (Late time-point): 1) Ischemic Core volume (volume of MRI-DWI lesion, relative cerebral blood flow<30% on CTP or hypodensity on NCCT), 2) Hypoperfusion lesion volume (ischemic lesion with time-to-maximum (Tmax)>6 sec on DSC-MRI and CTP), 3) Penumbra volume (perfusion volume-core volume). From these values, the following measures were constructed: 1) Early infarct core growth: Early infarct core volume-initial core volume; 2) Total infarct core growth: Late infarct core volume-initial core volume; 3) Early penumbra tissue proportion not advanced to ischemic core: 1—[(Early infarct core vol-Initial core vol)/Initial penumbra vol]; 4) Total Penumbral salvage proportion: 1—[(Late infarct core vol-Initial core vol)/Initial penumbra vol]; 5) Early hypoperfusion lesion volume change: Early hypoperfusion lesion volume-Initial hypoperfusion lesion volume; 6) Total hypoperfusion lesion volume change: Late hypoperfusion lesion volume-Initial hypoperfusion lesion volume.

Additional post-processing of early DSC-MRI and CTP images were performed for quantitative relative cerebral blood volume (qrCBV) analyses using Food and Drug Administration-approved software (Olea Sphere SP23; Olea Medical SAS, La Ciotat, France). Perfusion parametric maps of Tmax and rCBV were coregistered between the two scans using a 6-degree-of-freedom transformation and a mutual information cost function. Subsequently, a volume of interest (VOI) was generated from the visually perceptible perfusion abnormality on baseline Tmax maps, which was then automatically transferred over the coregistered follow-up perfusion maps. The CBV values within this VOI on baseline and follow-up scans were obtained using a voxel-based analysis. Finally, the CBV values were normalized to a region of interest placed in the contralateral centrum semiovale and mean rCBV was calculated for each scan.

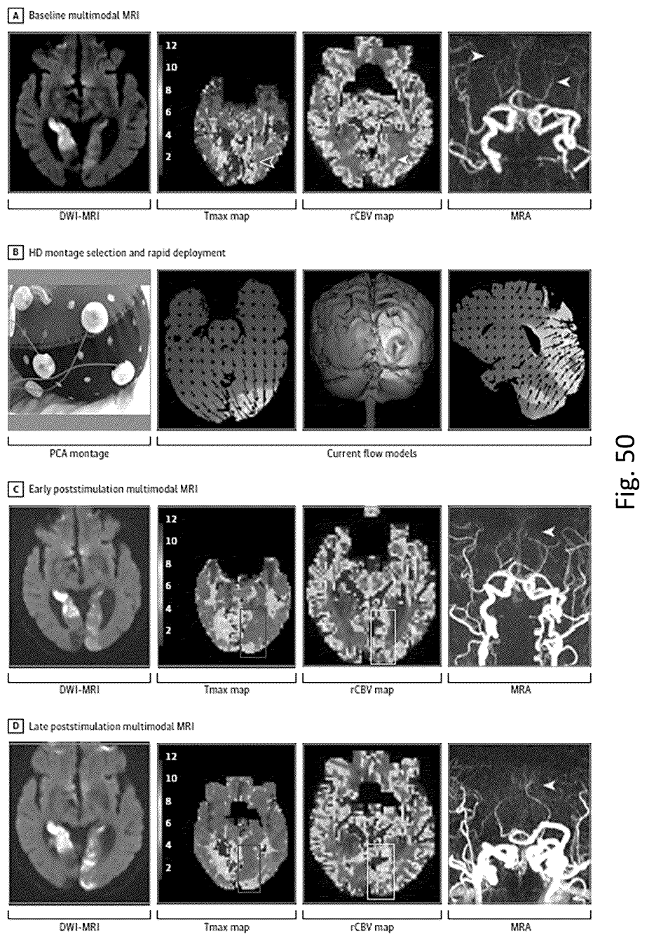

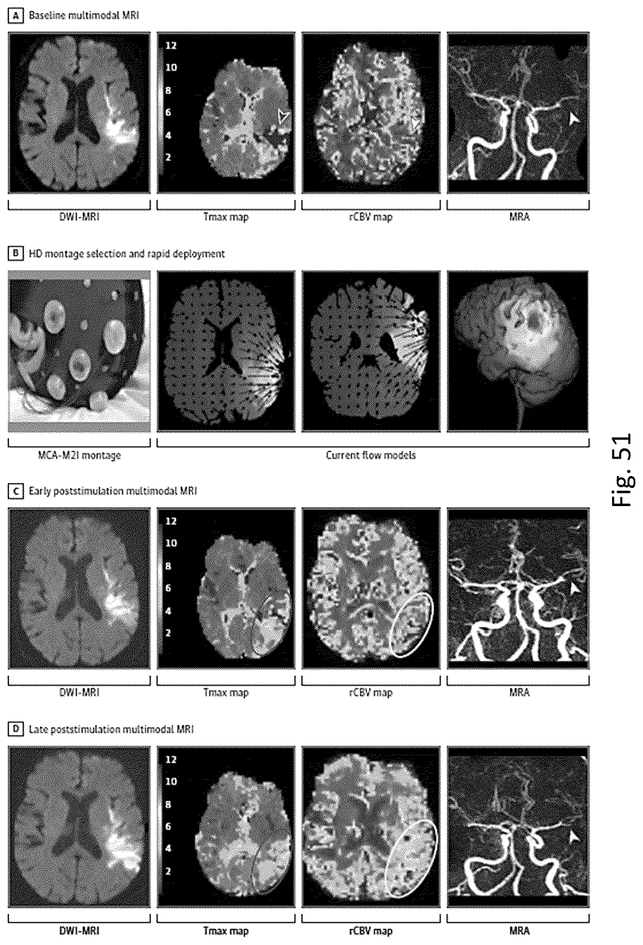

The flow of patients screening, enrollment, and follow-up is shown in FIG. 49. A total of 10 patients, 7 active and 3 sham, were enrolled in the UCLA Emergency Department, Neuro-Intensive Care and Stroke Units, from October 2018 to July 2021. The first 4 patients (3 active, 1 sham) were enrolled at dose tier 1 and received 1 milliamp (mA) of HD C-tDCS for 20 minutes; the subsequent 6 patients (4 active, 2 sham) were enrolled at tier 2 and received 2 mA of HD C-tDCS for 20 minutes. In active and sham patients mean (SD) age was 75 (20) years and 77 (12) years, the proportion of female patients was 7 of 10 (70%) and 1 of 3 (33%), and mean (SD) entry NIHSS was 8 (8.5) and 7 (2.6). Scans of exemplary patients are shown in FIGS. 50-51.

The speed of HD C-tDCS implementation was a median (IQR) 12.5 minutes (9-15 minutes) in the last 4 enrolled patients. The primary tolerability end point was met with all patients completing the assigned stimulation period. For the secondary tolerability end point, no discoloration or rash was detected on skin visual inspection after the stimulation. Only 1 tier 1 patient complained of mild skin burning, which was alleviated after a short pause of the stimulation.

One SICH occurred in 1 active patient at tier 2 of the study who was later determined to be a protocol deviation for not having fully met the entry criteria (stroke due to septic embolization). Nonetheless, tier 2 of the study was extended to enroll more patients. As recruitment was slower than expected, related to the COVID-19 pandemic, the study was stopped after enrollment of the 10th patient.

In the per-protocol exploratory efficacy analysis, imaging biomarkers of neuroprotection and collateral enhancement were characterized in 5 active patients and 3 sham patients, early and late poststimulation. The exploratory per protocol analysis of imaging end points excluded 2 active group patients with protocol deviations: 1 patient with no penumbra present at baseline on imaging core review and 1 with septic embolization as stroke cause.

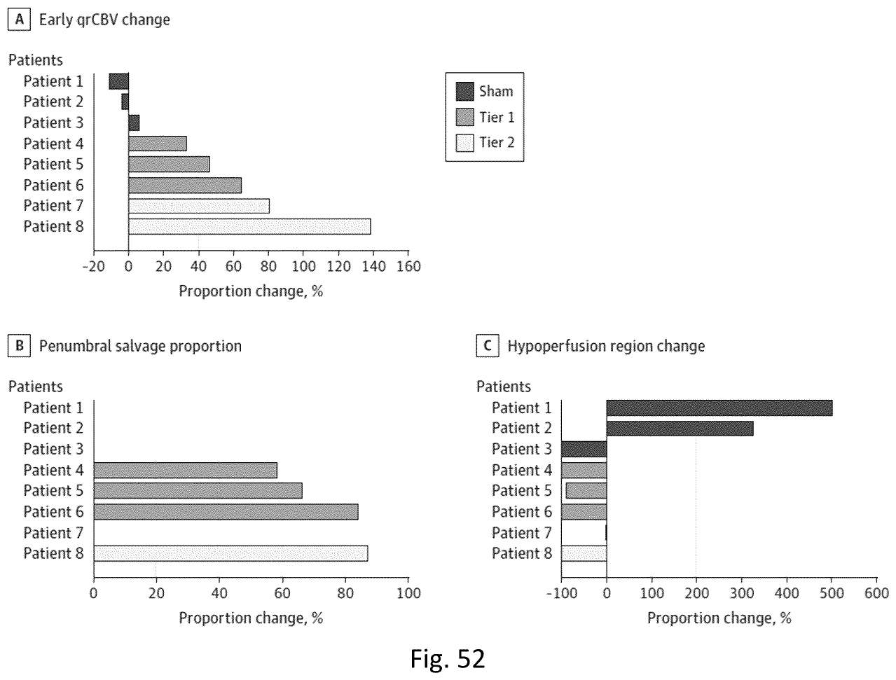

The hypoperfused region was reduced by a median (IQR) 100% (46% to 100%) in the active group vs increased by 325% (112% to 412%) in sham. Change in qrCBV early poststimulation was a median (IQR) 64% (40% to 110%) in active vs-4% (−7% to 1%) in sham patients. Penumbral salvage in the active C-tDCS group was a median (IQR) 66% (29% to 80.5%) compared with 0% (IQR 0% to 0%) in sham. For the imaging biomarker of collateral enhancement (qrCBV), the response was consistent with a dose-response effect, highest at tier 2, intermediate at tier 1, and lowest at sham (FIG. 52). The 24-hour vessel recanalization rate was 80% in active vs 33% in sham.

In a per-protocol exploratory clinical efficacy analysis, in the active group 3 patients had mRS between 0 and 2 and 2 had mRS of 3 at 90 days. Among the 3 sham patients, 2 had mRS between 0 and 2 and 1 an mRS of 6 at 90 days.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

The invention claimed is:

1. A method of providing therapy for acute ischemic strokes comprising the steps of:
positioning an electrical stimulation device comprising a cap comprising: a plurality of openings, a plurality of electrode holders and a plurality of electrodes onto a subject's head, wherein the plurality of electrodes are affixed to, embedded or otherwise integrated into plurality of electrode holders that is attached to the plurality of openings on the cap;
positioning the plurality of electrodes in a specified montage which corresponds to a therapeutic target region; and
providing a direct electrical current to the plurality of electrodes to salvage the subject's brain via reduction of an ischemic region volume thereby enhancing collaterals and promoting recanalization.

2. A method of providing therapy for acute ischemic strokes comprising the steps of:
positioning an electrical stimulation device comprising a cap comprising: a plurality of openings, a plurality of electrode holders and a plurality of electrodes onto a subject's head, wherein the plurality of electrodes are affixed to, embedded or otherwise integrated into plurality of electrode holders that is attached to the plurality of openings on the cap;
positioning the plurality of electrodes in a specified montage which corresponds to a therapeutic target region; and
providing a direct electrical current with a stimulation intensity ranging from 1 mA to 4 mA for 10 minutes to 60 minutes to the plurality of electrodes.

3. A method of treating a patient with acute ischemic strokes comprising the steps of:
positioning an electrical stimulation system comprising a cap configured with a plurality of electrodes onto a patient's head; and
activating the plurality of electrodes in a specified montage which corresponds to a therapeutic target region, wherein activating includes providing a direct electrical current to the plurality of electrodes to salvage the patient's brain.

4. (canceled)

5. (canceled)

6. (canceled)

7. The method of claim 1, wherein the montage is selected from the group consisting of: middle cerebral artery (MCA) M1 branch, MCA inferior branch (M2-I), MCA superior branch (M2-S), Anterior Cerebral artery (ACA), Posterior Cerebral Artery (PCA), Posterior Inferior Cerebellar Artery (PICA), and Internal Carotid Artery (ICA).

8. The method of claim 7, wherein the ACA montage is positioned proximate to the longitudinal fissure of the patient's brain, and proximate to the anterior portion of the patient's brain.

9. The method of claim 8, wherein the ACA montage comprises anodes over FPz, FCz, FC3, FP1, and cathode over F1 electrode locations.

10. The method of claim 7, wherein the MCA branch montage is positioned proximate to a central location between to the longitudinal fissure and outer edge of the patient's brain, and proximate to a central location between anterior and posterior portions of the patient's brain.

11. The method of claim 7, wherein the MCA branch montage comprises anodes over F3, Cz, P3, T7 and cathode over C3 electrode locations.

12. The method of claim 7, wherein the M2-S branch montage is positioned proximate to a central location between to the longitudinal fissure and outer edge of the patient's brain, and proximate the anterior portion of the patient's brain.

13. The method of claim 7, wherein the M2-S branch montage comprises anodes over C1, F1, F7, T7 and cathode over FC3 electrode locations.

14. The method of claim 7, wherein the M2-I branch montage is positioned proximate to a central location between to the longitudinal fissure and outer edge of the patient's brain, and proximate the posterior portion of the patient's brain.

15. The method of claim 7, wherein the M2-I branch montage comprises anodes over C3, P3, T7, P7 and cathode over CP5 electrode locations.

16. The method of claim 7, wherein the PCA montage is positioned proximate to the longitudinal fissure of the patient's brain, and proximate to the posterior portion of the patient's brain.

17. The method of claim 7, wherein the PCA montage comprises anodes over Pz, Iz, PO9, P3 and cathode over O1 electrode locations.

18. The method of claim 7, wherein the PICA montage is positioned proximate to the outer edge of the patient's brain, and proximate to the posterior portion of the patient's brain.

19. The method of claim 7, wherein the PICA montage comprises anodes over O1, P7, Ex1, EX5 and cathode over PO9 electrode locations.

20. The method of claim 7, wherein the ICA montage spans from proximate to the longitudinal fissure to proximate to the outer edge of the patient's brain, and proximate the anterior portion of the patient's brain.

21. The method of claim 7, wherein the ICA montage comprises anodes over the Fpz, Cz, CP5, F9 and cathode over F3 electrode locations.

22. The method of claim 1, wherein the direct electrical current is applied;
with a stimulation intensity ranging between 1 mA to 4 mA;
for a duration ranging between 10 to 60 minutes continuously; or
initially according to an up current ramp having a selected duration between 10 seconds to 10 minutes, and current application is terminated according to a down current ramp having a selected duration, wherein the selected duration between 10 seconds to 10 minutes.

23. (canceled)

24. (canceled)

25. (canceled)

26. (canceled)

27. (canceled)

28. (canceled)

29. (canceled)

30. (canceled)

31. (canceled)

32. The method of claim 1, wherein the ischemic region volume is reduced by 46% to 100%, an enhanced collateral comprises an increase of 40% to 110% of quantitative relative cerebral blood volume (qrCBV), or a rate of the recanalization is at least 80%.

33. (canceled)

34. (canceled)

35. (canceled)

36. (canceled)

37. (canceled)
